(19) United States
(12) Patent Application Publication
(10) Pub. No.: US 2025/0215052 A1
(43) Pub. Date: Jul. 3, 2025

(54) INHIBITORS OF PICK1 AND USES THEREOF

(71) Applicant: University of Copenhagen, Copenhagen K (DK)

(72) Inventors: Kenneth L. Madsen, Virum (DK); Kristian Strømgaard, Roskilde (DK); Ulrik Gether, Charlottenlund (DK); Anders Bach, Valby (DK); Nikolaj Riis Christensen, Allerød (DK)

(73) Assignee: University of Copenhagen, Copenhagen K (DK)

(21) Appl. No.: 19/012,443

(22) Filed: Jan. 7, 2025

Related U.S. Application Data
(62) Division of application No. 17/283,997, filed on Apr. 9, 2021, now abandoned.

(30) Foreign Application Priority Data
Oct. 22, 2018 (EP) 18201731.9

(51) Int. Cl. C07K 7/06; A61K 38/00; A61P 25/00

(52) U.S. Cl. CPC C07K 7/06; A61P 25/00; A61K 38/00; C07K 2319/10

(57) ABSTRACT

The present disclosure relates to peptides and peptide analogues with high affinity for the PDZ domains of PICK1. The peptide or peptide analogue interacts with PICK1, blocking the native protein-protein interactions between PICK1 and its natural ligands. The disclosure furthermore relates to the therapeutic use of these peptides and peptide analogues in prevention and/or treatment of diseases and disorders associated with maladaptive plasticity, drug addiction and neuropathic pain.

### CROSS REFERENCE TO RELATED APPLICATIONS

This application is a division of U.S. patent application Ser. No. 17/283,997, filed Apr. 9, 2021, which is a U.S. National Stage of International Application PCT/EP2019/078716 filed Oct. 22, 2019, which claims priority to European Application No: 18201731.9 filed Oct. 22, 2018, the entire contents of each of which are incorporated herein by reference.

### SEQUENCE LISTING

A computer-readable form (CRF) sequence listing having file name having file name 16COPE-HO78203VA.xml (27,715 bytes), created on Jan. 7, 2024, is incorporated herein by reference. The nucleic and amino acid sequences listed in the accompanying sequence listing are shown using standard abbreviations as defined in 37 C.F.R. § 1.822.

### TECHNICAL FIELD

The present invention relates to peptides which bind to Protein Interacting with C Kinase-1 (PICK1) and thereby block PICK1-mediated protein-protein interactions. The invention furthermore relates to therapeutic and diagnostic use of said peptides.

### BACKGROUND

Protein-protein interactions (PPIs) are vital for most biochemical and cellular processes and are often mediated by scaffold and signal transduction complexes. One of the most abundant classes of human facilitators of PPIs is the family of postsynaptic density protein-95 (PSD-95)/Discs-large/ZO-1 (PDZ) domains. Protein Interacting with C Kinase-1 (PICK1) is an intracellular scaffold protein primarily involved in regulation of protein trafficking and cell migration by mediating and facilitating PPIs via its two PDZ domains. Central to PICK1′s cellular role is its ability to bind and interact with numerous intracellular molecules including various protein partners, as well as membrane phospholipids. PDZ domain proteins in the postsynaptic density, which dynamically regulate the surface expression and activity of the glutamate receptors, represent attractive alternative drug targets, but it has proven challenging to develop sufficiently potent small molecule inhibitors and peptide drugs generally suffer from poor pharmacokinetic profiles. PICK1 is another PDZ domain containing scaffolding protein that plays a central role in synaptic plasticity. PICK1 is a functional dimer, with two PDZ domains flanking the central membrane binding BAR domain, which also mediates the dimerization. The PICK1 PDZ domain interacts directly with the C-terminus of the GluA2 subunit of the AMPA receptors (AMPAR) as well as protein kinase A and C, thereby regulating AMPAR phosphorylation and surface expression and in turn synaptic plasticity tuning the efficacy of individual synapses.

Synaptic plasticity serves as the molecular substrate for learning and memory. In diseased states, such as ischemia after stroke, neuropathic pain and addiction, abnormal synaptic stimulation causes maladaptive plasticity leading to hyper-sensitization of glutamatergic synapses through expression of calcium permeable (CP) AMPA-type glutamate receptors (CP-AMPARs).

Although numerous diseased states, including ischemia after stroke and head injury, amyotrophic lateral sclerosis (ALS), epilepsy, neuropathic pain and addiction, involve an over-activation or sensitization of the glutamate system, the NMDA receptor antagonists such as ketamine (anaesthetic) are, due to general problems with severe side effects, currently the only drugs in clinical use that target the glutamate system. There is thus a need for a treatment for diseases such as neuropathic pain, excitotoxicity following ischemia and drug addiction, three conditions that are currently without any effective therapy.

### SUMMARY

The present invention provides a high affinity peptide inhibitor towards the scaffolding protein, protein interacting C kinase 1 (PICK1), which is known to be responsible for the abnormal expression of certain AMPA receptor subtypes via regulating their trafficking. This invention differs from current glutamate receptor drugs by targeting the scaffolding proteins responsible for the trafficking of the receptor, rather than targeting the receptor directly, thus reducing possible side effects of the compound in patients with conditions such as neuropathic pain, excitotoxicity following ischemia or drug addiction.

In one aspect, the present invention relates to a compound comprising:
a) a first peptide comprising an amino acid sequence of the general formula: X₁X₂X₃X₄X₅ (SEQ ID NO:2);
b) a second peptide comprising an amino acid sequence of the general formula: X₁X₂X₃X₄X₅ (SEQ ID NO:2);
   wherein:
   X₁ is a proteogenic or non-proteogenic amino acid, preferably H, L, I, or A; or is absent;
   X₂ is a proteogenic or non-proteogenic amino acid, preferably W, or F; or is absent;
   X₃ is a proteogenic or non-proteogenic amino acid, preferably L, V, I, F; A, or Y;
   X₄ is a proteogenic or non-proteogenic amino acid, preferably K, or R; and
   X₅ is V, I or C; and
c) an NPEG linker linking the first peptide to the second peptide; and
d) a Cell Penetrating Peptide (CPP).

In one embodiment, the compound is a PICK1 inhibitor.

Thus, in one aspect, the present invention relates to a PICK1 inhibitor comprising:
a) a first peptide comprising an amino acid sequence of the general formula: X₁X₂X₃X₄X₅ (SEQ ID NO:2);
b) a second peptide comprising an amino acid sequence of the general formula: X₁X₂X₃X₄X₅ (SEQ ID NO:2); wherein:
   X₁ is a proteogenic or non-proteogenic amino acid, preferably H, L, I, or A; or is absent;
   X₂ is a proteogenic or non-proteogenic amino acid, preferably W, or F; or is absent;
   X₃ is a proteogenic or non-proteogenic amino acid, preferably L, V, I, F; A, or Y;
   X₄ is a proteogenic or non-proteogenic amino acid, preferably K, or R;
   and X₅ is V, I or C; and
c) an NPEG linker linking the first peptide to the second peptide; and
d) a Cell Penetrating Peptide (CPP).

In another aspect, the present invention provides a composition comprising the compound according to the above aspect.

In another aspect, the present invention provides a composition comprising the compound according to any of the above aspects of the invention for use as a medicament.

In another aspect, the present invention provides a composition or compound according to any of the above aspects of the invention for use in the prophylaxis and/or treatment of diseases and/or disorders associated with maladaptive plasticity in a subject.

In another aspect, the present invention provides a method of providing prophylaxis and/or treatment of diseases and/or disorders associated with maladaptive plasticity in a subject, comprising administering the compound or composition according to any of the above aspects of the invention to the subject.

In another aspect the present invention relates to the use of the composition or compound according to any of above aspects for the manufacture of a medicament for the treatment of diseases and/or disorders associated with maladaptive plasticity.

### DESCRIPTION OF DRAWINGS

FIG. 1: Development of a high affinity bivalent inhibitor of PICK1

(A-D) Fluorescent polarization experiments to determine affinities of peptides for PICK in solution. (A) Fold affinity change of DAT C truncations (C13-C3; SEQ ID NO: 24, 23, 22, 21, 20, 19, 18, 17, 1, 16 and 15). (B) Fold affinity change upon PEGx dimerization of C5. (C) Saturation binding of fluorescently labelled 5FAM-C5, 5FAM-TAT11-C5, TMR-TAT11-C5 and TMR-TAT11-P4-(C5)₂. (D) Affinity of respective tracer peptide and C5 (SEQ ID NO: 1), TAT11-C5 (SEQ ID NO: 7 linked to SEQ ID NO: 1) and TAT11-P4-(C5)₂. (E) Primary sequence and affinity of C5, TAT11-C5 and TAT11-P4-(C5)₂. TMR: TAMRA, OrG: OregonGreen.

FIG. 2: TAT-P4-(C5)2 but not TAT-(C5) can dissociate PICK1 complexes with transmembrane interaction partners

(A) Graphical illustration of supported cell membrane sheet (SCMS) assay. (B) Representative confocal images of SCMS expressing DATC24, incubated with fluorescently labelled PICK1 and subsequently unlabelled peptide. (C) SCMS derived competitive action dashed vertical line indicates 10 μM for which statistical analysis is carried out. (D) Illustration of experimental approach.

FIG. 3: TAT-P4 (C5)2 but not TAT-(C5) reconfigures PICK1 into an oligomeric state. (A-C) Size exclusion chromatography (SEC) profiles of PICK1 in absence (black, Absmax11.7 ml) and presence of (A) TAT-(C5) (grey, Absmax=11.7 ml), (B) TAT-P4-(C5)2 (grey, Absmax=10.7 ml) or (C) fluorescent TMR-TAT-P4-(C5)2 (dashed (Abs280 nm), full line (abs544 nm), Absmax=10.7ml).

FIG. 4: TAT-P4(C5)2 induces a non-native tetrameric configuration of PICK1 (A) Small angle X-ray scattering concentration curves of PICK1 in absence (black) and presence of TAT-P4-(C5)2 (Gray). (B) EOM ensemble for PICK1 in complex with TAT-4(C5)2 with shading according to model ensemble percentage (9/18/27/45%.). N-and C-terminal unstructured regions are removed for visual clarity. (C) Graphical representation of the non-native PICK1 tetramer induced by TAT-P4 (C5)2.

FIG. 5: TAMRA-conjugated TAT-P4-(C5)2 and TAT-(C5) penetrate cultured hippocampal neurons.

Representative confocal images of hippocampal neurons illustrating the penetration of 5 μM CPP-coupled peptides TMR-TAT-P4-(C5)2 (upper panel) and TMR-TAT-(C5) (middle panel), but not the control TMR-C5 (lower panel). The cell membrane is stained with DiO. Scale bar: 10 μm.

FIG. 6: In vivo target engagement of TAT-P4-(C5)2 peptide Immunoblot of co-immunoprecipitated (IP) PICK1: Biotin-TAT-P4-(C5)2 from lumbar spinal cord total lysates testifies in vivo target engagement following 20 μM intrathecal (i.t.) injection, compared to the control Biotin-TAT peptide.

FIG. 7: TAT-P4-(C5)2 disrupts PICK: GluA2 interaction in vivo (a,b) Representative Immunoblots of co-immunoprecipitated (IP) PICK1:GluA2 from spinal cord lumbar tract total lysates demonstrate partial disruption of this interaction 1 hour after 20 μM i.t. injection of (a) TAT-P4-(C5)2 but not (b) TAT-(C5). (bottom bar charts) Densitometric analysis of immunoblots indicate a significant effect only following TAT-P4-(C5)2 treatment.

FIG. 8: Differential effects of TAT-P4-(C5)2 and TAT-(C5) on spinal cord GluA1 and GluA2 surface level under basal and injured conditions.

(a) Surface biotinylation of spinal cord slices from naïve mice under basal condition demonstrates a reduction of GluA2 surface level upon TAT-P4-(C5)2 and TAT-(C5) peptides incubation, while preserving surface-expressed GluA1, compared to the untreated condition (CTR). (bottom bar charts) Densitometric analysis of immunoblots shows a tendency in the reduction of GluA2 surface level.

FIG. 9: TAT-P4-(C5)2 effectively alleviates mechanical hyperalgesia in the neuropathic pain acute phase.

Acute phase Von Frey test following i.t. injection reveals reduced ipsilateral paw hypersensitivity of SNI mice from 1 up to 3 hours following 20 μM (a) TAT-P4-(C5)2, (b) but not TAT-(C5) compared to time 0 per-injection point. (c) Surface biotinylation of spinal cord slices reveals up-regulation of both GluA1 and GluA2 surface level following spared nerve injury (SNI) surgery compared to non-operated animal control. (Bottom bar charts) Densitometric analysis of immunoblots indicates that TAT-P4-(C5)2 but not TAT-(C5) significantly reduce the SNI-induced GluA2 surface upregulation and shows a strong tendency for GluA1 as well.

FIG. 10: TAT-P4-(C5)2 alleviates mechanical hyperalgesia in the neuropathic pain chronic phase.

Von Frey test in chronic phase SNI mice shows significant pain reduction in both animal genders up to 3 hours following 20 μM TAT-P4-(C5)2 i.t. injection, compared to time 0 pre-injection. Von Frey test in chronic phase (day 14 after surgery) shows full recovery from mechanical hypersensitivity at 2 hours following intraperitoneal injection (i.p) of 30 mg/kg gabapentin in both genders SNI animal.

FIG. 11: Systemic administration of TAT-P4-(C5)2 dose-dependently attenuates cocaine priming-induced reinstatement of drug-seeking behavior in rats. (A) Cocaine seeking was significantly decreased in rats pretreated with 3.0 nmol/g TAT-P4-(C5)2 intravenously (i.v.) compared to vehicle-treated controls. (B) Dose-dependent effects of TAT11-N-DATC5 were tested in a separate cohort of rats. Cocaine seeking was significantly reduced in rats pretreated with 3.0 nmol/g TAT-P4-(C5)2 (i.v.) compared to vehicle-treated controls and rats pretreated with 0.3 nmol/g TAT-P4-(C5)2. No effects of TAT-P4-(C5)2 on inactive lever responding were found in either experiment.

FIG. 12: Systemic administration of TAT-P4-(C5)2 does not affect sucrose seeking or locomotor activity in rats. (A) There were no effects of 3.0 nmol/g TAT-P4-(C5)2 (i.v.)

on the reinstatement of sucrose-seeking behavior. Moreover, systemic TAT-P4-(C5)2 did not affect locomotor activity in cocaine-experienced rats.

FIG. 13: TAMRA-conjugated TAT-P4-(C5)2 administered systemically crosses the blood brain barrier and is visualized in the nucleus accumbens shell of rats Separate cocaine-experienced rats were injected with TAMRA-conjugated TAT-P4-(C5) 2 (3.0 nmol/g, i.v.) during extinction. Representative confocal images reveal TAMRA-conjugated TAT-P4-(C5)2 (small pointed structures) located in proximity to neurons labeled with NeuN (bright nuclear fluorescence) in the nucleus accumbens shell 15 (A), 30 (C) and 90 (E) minutes post infusion. Images are compressed z-stacks with a 0.5 μm step size (scale bar: 25 μm).

FIG. 14: Schematic structure of peptides with alternative CPPs; TAT-P₄-(C5)₂, PNT-P₄-(C5)₂, TP10-P₄-(C5)₂, and MAP-P₄-(C5)₂. C5: HWLKV (SEQ ID NO: 1),

TAT:
(SEQ ID NO: 7)
YGRKKRRQRRR,
PNT:
(SEQ ID NO: 12)
RQIKIWFQNRRMKWKK,
TP10:
(SEQ ID NO: 13)
GWTLNSAGYLLGKINLKALAALAKKIL,
and
MAP:
(SEQ ID NO: 14)
KLALKLALKALKAALKLA

FIG. 15: Fluorescence polarization competition binding curves for the unlabelled peptides. A fixed concentration of PICK1 (0.25 μM) and tracer 5-FAM-TAT-DATC5 (5 nM) was titrated with increasing concentration of the unlabelled peptides. This caused a displacement of the fluorescently labelled molecule (tracer) with the unlabelled peptides, and gave rise to decrease in the polarization value (mP). Data expressed as mean±SEM (n=3).

FIG. 16: FPLC Size Exclusion chromatography (SEC) runs for different CPP variants. Each sample was diluted into PBS and analyzed on a Superdex200 Increase 10/300 24 ml column. Elution profiles were measured as the absorbance at 280 nm and each trace was normalized for simple comparison of the main peak elution volume.

FIG. 17: Overview of the experimental setup for studying the analgesic effect of the CPP-peptides in the SNI pain model

FIG. 18: The analgesic effect of the peptides in the SNI pain model after subcutaneous administration. The analgesic effect on SNI mice after s.c administration (10 μmol/kg) of the 4 different peptides. Both ipsilateral and contralateral 50% PWT (g) are plotted on a log Y-scale for each Von Frey test performed at different time points (Baseline, Before drug, +1 h, +2 h, +3 h and +4 h). Before drug 50% PWT (g) is compared to 1 h, 2 h, 3 h and 4 h after drug injection for the ipsilateral paw and contralateral paw. n=8 mice/time point. Two-way RM ANOVA, Dunnett's multiple comparison test performed using Graphpad Prism 7.0 for each time point against before drug. (p<0.05: *, p<0.01: **, p<0.001: ***, p<0.0001: ****). All data plotted as mean±SEM

FIG. 19: The analgesic effect of the peptides in the SNI pain model after subcutaneous administration. The analgesic effect on SNI mice after s.c administration (10 μmol/kg) of 4 different peptide treatments (TAT, PNT, TP10 and MAP) compared with vehicle treatment. The 50% PWT (g) for the ipsilateral paw is plotted on a log Y-scale for each Von Frey test performed at different timepoints (Baseline, Before drug (D+7), +1 h, +2 h, +3 h and +4 h). n=8 mice/peptide, n=7 mice/vehicle. Two-way RM ANOVA, Bonferroni post hoc correction performed using Graphpad Prism 7.0 against saline treatment at the given time point (p<0.05: *, p<0.01: **, p<0.001: ***, p<0.0001: ****). All data plotted as mean±SEM

FIG. 20: Chemical structure of tested dimeric PICK1 inhibitors without the CPP-residue, but with different PEGx linkers (x=0-4 residues) linked to the N-terminal amine of HWLKV (SEQ ID NO: 1; C5) (PEG0-(C5)₂, PEG1-(C5)₂, PEG2-(C5)₂, PEG3-(C5)₂, PEG4-(C5)₂).

FIG. 21: Fold affinity gain over monomeric C5 (HWLKV; SEQ ID NO: 1) peptide for various PEG linker compounds towards purified PICK1 as determined by fluorescence polarization competition binding.

FIG. 22: Effect of single amino acid substitutions in DAT C5 (HWLKV: SEQ ID NO: 1) on binding affinity. A library of 12 C5 peptides with single amino acids substitutions in position X₁-X₅ of the sequence HWLKV was tested in fluorescence polarization binding in competition with fluorescently labelled DATC5. Values are given as fold change compared to the reference peptide HWLKV (set to 1).

FIG. 23: Plasma and PBS stability of DAT-C5 (C5 only), TAT11-C5 (monomer) and TAT-P₄-(C5)₂ (dimer) at indicated time points.

FIG. 24: Pain relief by TAT-P4-(C5)₂ in the CFA model of inflammatory pain. Assessment of pain, measured as paw withdrawal threshold, was determine before CFA administration (day-2), after CFA administration (−1 hr), and then following i.t administration of 20 uM TAT-P4-(C5)₂ (1 hr, 5 hrs, 24 hrs).

### DETAILED DESCRIPTION

### Definition of Abbreviations and Terms

Amino acids, that are proteinogenic are named herein using either its 1-letter or 3-letter code according to the recommendations from IUPAC, see for example www.chem.qmw.ac.uk/iupac. If nothing else is specified an amino acid may be of D or L-form. In the description (but not in the sequence listing) a 3-letter code starting with a capital letter indicates an amino acid of L-form, whereas a 3-letter code in small letters indicates an amino acid of D-form;
CNS, central nervous system;
CPP, cell penetrating peptide; refers to a peptide characterized by the ability to cross the plasma membrane of mammalian cells, and thereby may give rise to the intracellular delivery of cargo molecules, such as peptides, proteins, oligonucleotides to which it is linked;
Ethylene glycol moiety, here refers to the structural unit that constitute a PEG or NPEG linker. A more technical name of a ‘ethylene glycol moiety’ is ‘oxyethylene’, and the chemical formula of the unit is here shown:

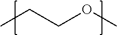

NPEG, is the novel linker type described herein, which is derived from the classical PEG linker, but where one or more of the backbone oxygen atoms is replaced with a nitrogen atom. NPEG, PEG and P are used herein interchangeably and refer to the NPEG linker;
PDZ, acronym combining the first letters of the first three proteins discovered to share the domain Postsynaptic density protein-95 (PSD-95), Drosophila homologue discs large tumor suppressor (DlgA), and Zonula occludens-1 protein (zo-1). PDZ domains are common structural domains of 80-90 amino-acids found in signaling proteins. Proteins containing PDZ domains often play a key role in anchoring receptor proteins in the membrane to cytoskeletal components.
PEG, polyethylene glycol; PEG is a polymer of ethylene glycol_having the chemical formula C₂ₙ₊₂H₄ₙ₊₆Oₙ₊₂, and the repeating structure:

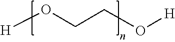

where for example 12 PEG moieties, PEG₁₂, P₁₂ or PEG12, corresponds to a polymer of 12 ethylene glycol moieties (x=12);
Retroinverso, retroinverso peptides are composed of D-amino acids assembled in the reverse order from that of the parent L-amino acid sequence;
Retroinverso-D-Tat sequence, a 9-mer CPP sequence made by reverting the Tat sequence and using D-amino acids (rrrqrrkkr) (SEQ ID NO: 10), which facilitates permeability across biological membranes, including the blood-brain barrier, and whose structure renders it stable to protease enzymes;
Tat sequence, or TAT11 is an 11-mer CPP sequence (YGRKKRRQRRR) (SEQ ID NO: 7) derived from the human immunodeficiency virus-type 1 (HIV-1) Tat protein, which facilitates permeability across biological membranes, including the blood-brain barrier;
Penetratin (PNT) is a 16 a.a. sequence, derived from a 60 a.a. residue Antennapedia homeodomain (DHAntp) from Drosophilia. This 16 a.a. sequence, belonging to the third a-helix of the DHAntp, corresponding to residue 43-58, is the domain facilitating transporting across the membrane.
TP10 is a truncated analogue of Transportan, synthesized by deletion of six a.a. from the N-terminus. The internalization mechanism of TP10 is suggested to involve peptide binding to the cellular surface, creating a local positive curvature and a mass imbalance across the bilayer which strains the membrane resulting in pore formation and finally translocation across the membrane.
MAP the “Model of Amphipathic Helix” is an artificial sequence and designed to have cell-penetrating properties.
Amide bond is formed by a reaction between a carboxylic acid and an amine (and concomitant elimination of water). Where the reaction is between two amino acid residues, the bond formed as a result of the reaction is known as a peptide linkage (peptide bond).
Von Frey test, assess touch sensitivity with von Frey filaments. These filaments are applied to the underside of the paw after the mouse has settled into a comfortable position within a restricted area that has a perforated floor. The filaments are calibrated to flex when the set force is applied to the paw. Filaments are presented in order of increasing stiffness, until a paw withdrawal is detected.
Absent is to be understood as that the amino acid residues directly adjacent to the absent amino acid are directly linked to each other by a conventional amide bond.
AMPAR or AMPA receptor or AMPA-type glutamate receptor is an ionotropic transmembrane receptor for glutamate that mediates fast synaptic transmission in the central nervous system (CNS). PICK1 interacts with AMPAR via the PDZ domain.

### Chemical Structure of CPP-Containing Dimeric PICK1 Inhibitors

The invention provides a dimeric PICK1 inhibitor comprising a first peptide consisting of the amino acid sequence HWLKV (SEQ ID NO: 1) linked by an NPEG-linker to a second peptide consisting of the amino acid sequence HWLKV (SEQ ID NO: 1) wherein the compound is further linked to a Cell Penetrating Peptide (CPP).

The invention provides a compound comprising:
a) a first peptide comprising an amino acid sequence of the general formula: X₁X₂X₃X₄X₅ (SEQ ID NO:2);
b) a second peptide comprising an amino acid sequence of the general formula: X₁X₂X₃X₄X₅ (SEQ ID NO:2);
   wherein:
   X₁ is a proteogenic or non-proteogenic amino acid, preferably H, L, I, A; or is absent;
   X₂ is a proteogenic or non-proteogenic amino acid, preferably W, F; or is absent;
   X₃ is a proteogenic or non-proteogenic amino acid, preferably is L, V, I, F, A, Y;
   X₄ is a proteogenic or non-proteogenic amino acid, preferably is K, R;
   X₅ is V, I or C; and
c) an NPEG linker linking the first peptide to the second peptide; and
d) a Cell Penetrating Peptide (CPP).

In one embodiment, the compound is a PICK 1 inhibitor.

Thus, the present disclosure provides a PICK1 inhibitor comprising:
a) a first peptide comprising an amino acid sequence of the general formula:

(SEQ ID NO: 2)
X₁X₂X₃X₄X₅;

b) a second peptide comprising an amino acid sequence of the general formula: X₁X₂X₃X₄X₅ (SEQ ID NO:2);
   wherein:
   X₁ is a proteogenic or non-proteogenic amino acid, preferably H, L, I, A; or is absent;
   X₂ is a proteogenic or non-proteogenic amino acid, preferably W, F; or is absent;
   X₃ is a proteogenic or non-proteogenic amino acid, preferably is L, V, I, F; A, Y;
   X₄ is a proteogenic or non-proteogenic amino acid, preferably is K, R;
   X₅ is V, I or C; and
c) an NPEG linker linking the first peptide to the second peptide; and
d) a Cell Penetrating Peptide (CPP).

The term ‘absent’ as used herein, e.g. “X₁ is any proteogenic or non proteogenic amino acid, preferably H, L, I, or A; or is absent” is to be understood as that the amino acid residues directly adjacent to the absent amino acid are directly bonded to each other by a conventional amide bond.

“Proteogenic” as used herein refers to the 20 amino acids that constitute all proteins that are naturally occurring. “Non-proteogenic” amino acid broadly refers to any amino acid which is not capable of being incorporated into peptides or proteins by a living organism and includes non-natural amino acids.

In one embodiment, the first and/or the second peptide comprise or consist of the amino acid sequence X₁X₂X₃X₄X₅ (SEQ ID NO:3);
wherein:
X₁ is H, L, I, A; or is absent;
X₂ is W, F; or is absent;
X₃ is L, V, I, F, A, or Y;
X₄ is K or R; and
X₅ is V, I or C.

In one embodiment, the first and/or the second peptide comprise or consist of the amino acid sequence X₁X₂X₃X₄X₅ (SEQ ID NO:4);
wherein:
X₁ is H, L, I, or A;
X₂ is W or F;
X₃ is L, V, I, F; A, or Y;
X₄ is K or R; and
X₅ is V, I or C.

In one embodiment, the first and/or the second peptide comprise or consist of the amino acid sequence X₁X₂X₃X₄X₅ (SEQ ID NO:5);
wherein:
X₁ is H, I, or A;
X₂ is W or F;
X₃ is L, I or V;
X₄ is K or R; and
X₅ is V or C.

In one embodiment, the first and/or the second peptide comprise or consist of the amino acid sequence X₁X₂X₃X₄X₅ (SEQ ID NO:6);
wherein:
X₁ is H or A;
X₂ is W;
X₃ is L, I or V;
X₄ is K or R;
and X₅ is V.

In one embodiment, the first and/or the second peptide comprise or consist of the amino acid sequence HWLKV (SEQ ID NO: 1).

In one embodiment, the first and/or the second peptide further comprises an arginine residue attached via an amide bond to the N-terminus, thereby forming a hexapeptide having the amino acid sequence of RX₁X₂X₃X₄X₅, wherein X₁-X₅ is SEQ ID NOs: 2, 3, 4, 5, 6.

In one embodiment, the first and/or the second peptide further comprises a LR dipeptide attached via an amide bond to the N-terminus, thereby forming a heptapeptide having the amino acid sequence of LRX₁X₂X₃X₄X₅, wherein X₁-X₅ is SEQ ID NOs: 2, 3, 4, 5, 6.

The invention further provides a compound that has the generic structure of formula:

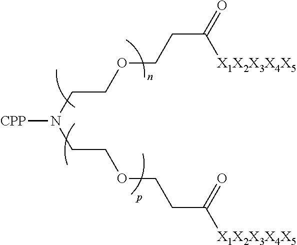

wherein
n is an integer 2 to 12;
p is an integer 2, to 12;
CPP is a cell penetrating peptide,
wherein X₁-X₅ is SEQ ID NOs: 2, 3, 4, 5, 6.

In one embodiment, the compound has the generic structure of formula:

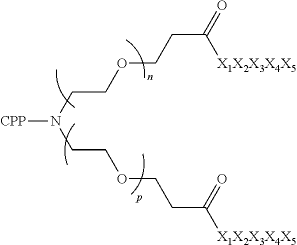

wherein
n is an integer 0 to 12;
p is an integer 0, to 12;
CPP is a cell penetrating peptide,
wherein X₁-X₅ is SEQ ID NOs: 2, 3, 4, 5, 6.

In another embodiment, the said compound that has the generic structure of formula:

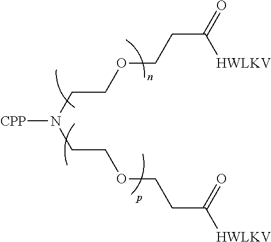

wherein
n is an integer 2 to 12;
p is an integer 2, to 12;
CPP is a cell penetrating peptide,
wherein HWLKV is SEQ ID NO: 1.

In one embodiment, the compound has the generic structure of formula:

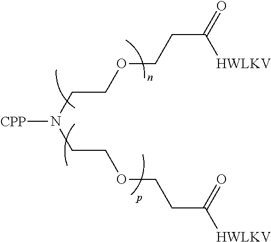

wherein
n is an integer 0 to 12;
p is an integer 0, to 12;
CPP is a cell penetrating peptide
wherein HWLKV is SEQ ID NO: 1.

The said linker comprises a derivative of a PEG linker, termed NPEG, wherein one oxygen atom in the backbone of the PEG linker is replaced with a nitrogen atom. The nitrogen atom may be substituted for any one of the oxygen atoms in the backbone of the PEG linker. The carbonyl groups of the NPEG linker are linked to the first and second peptide or peptide analogue respectively, preferably where the link is an amide bond to a terminal residue of the peptide or peptide analogue.

In one embodiment, the NPEG-linker comprises in the range of 0 to 12 ethylene glycol moieties wherein one or more of the backbone oxygen atoms is replaced with a nitrogen atom, such as in the range of 0 to 10, for example in the range of 0 to 8, such as in the range of 0 to 6, for example in the range of 0 to 4, for example in the range of 2 to 12, such as in the range of 2 to 10, for example in the range of 2 to 8, such as in the range of 2 to 6, for example in the range of 2 to 4 ethylene glycol moieties wherein one or more of the backbone oxygen atoms is replaced with a nitrogen atom. In a preferred embodiment, the NPEG-linker comprises 4 ethylene glycol moieties wherein one or more of the backbone oxygen atoms is replaced with a nitrogen atom.

In one embodiment, the NPEG-linker has one backbone oxygen replaced with a nitrogen atom.

In one embodiment, the NPEG-linker comprises a carboxylic acid in each end, such as a 2-carboxyethyl at each end in order to facilitate the generation of a amide bond to the N-terminus of the PICK1 binding peptide.

In one embodiment, the one or more nitrogen atom of the NPEG-linker is positioned at any position along the NPEG-linker, such as for example positioned in the middle of the NPEG-linker or positioned towards one end of the NPEG-linker.

In one embodiment, the NPEG-linker is conjugated to the first and second peptide via an amide bond formed between the carboxylic acids of the NPEG-linker and the N-terminus of the first and second peptides.

In one embodiment, the linker comprises 2 to 12 ethylene glycol moieties (x=2-12). In one embodiment, the linker comprises 4 moieties of ethylene glycol (x=4).

In one embodiment, the compound is selected from the group consisting of Tat-NPEG₄-(HWLKV)₂, TP10-NPEG₄-(HWLKV)₂, and MAP-NPEG₄-(HWLKV)₂ (HWLKV: SEQ ID NO: 1).

In another embodiment, said compound is selected from the group consisting of Tat-PEG₄ (HWLKV)₂ and Tat-NPEG₄(HWLKV)₂ (HWLKV: SEQ ID NO: 1).

In one embodiment, the CPP is linked to the nitrogen atom of the linker by an amide bond. The CPP may be any CPP know in the art that has the ability to translocate the plasma membrane and facilitate the delivery of the compound of the present invention to the cytoplasm or an organelle of a cell. In one embodiment, the CPP comprises a TAT peptide (SEQ ID NO: 7), a Retroinverso-D-TAT peptide (SEQ ID NO: 10), a polyarginine peptide (SEQ ID NO: 11), a PNT peptide (SEQ ID NO: 12), a TP10 peptide (SEQ ID NO: 13) or a MAP peptide (SEQ ID NO: 14). In another embodiment, the CPP comprises a Tat peptide, a Retroinverso-D-Tat peptide or a polyarginine peptide. In one embodiment, the CPP comprises a TP10 peptide or a MAP peptide. In yet another embodiment, the CPP is a Tat peptide having amino acid sequence YGRKKRRQRRR (SEQ ID NO: 7) or a Retroinverso-d-Tat peptide having amino acid sequence of rrrqrrkkr (SEQ ID NO: 10). In another embodiment, the CPP comprises or consists of the amino acid sequence RKKRRQRRR (SEQ ID NO: 8). In yet another embodiment, the CPP comprises or consists of the amino acid sequence GRKKRRQRRRP (SEQ ID NO: 9).

The dimeric PICK1 inhibitor, according to the first aspect comprises a CPP that is linked to the inhibitor via a chemical bond either directly or indirectly to the nitrogen atom in the backbone of the NPEG linker, where the nitrogen atom can be symmetrically- or asymmetrically-positioned in the linker. Linkage of the CPP to the nitrogen of the NPEG linker may be mediated via an amide bond, a maleimide coupling, a disulfide bond, or amino-reactive electrophilic groups, selected from among N-hydroxysuccinimide (NHS) ester, p-nitrophenyl ester, succinimidyl carbonate, p-nitrophenyl carbonate, succinimidyl urethane, isocyanate, isothiocyanate, acyl azide, sulfonyl chloride, aldehyde, carbonate, imidioester or anhydride; and thio-reactive groups selected from among haloacetyl, alkyl halide derivatives, aziridine, acryloyl derivatives arylating agents.

Alternatively, linkage of the CPP to the nitrogen of the linker may be mediated via a spacer group, where a suitable spacer group can for example be any amino acid such as cysteine, glycine, alanine; short alkane chains or short PEG/NPEG chains.

In one embodiment, the dimeric inhibitor has a linker according to the first aspect of the present invention, wherein the linker links two peptides of the amino acid sequence ID NO: 2. In one embodiment, the dimeric inhibitor has a linker that links two pentapeptides of the sequence HWLKV (SEQ ID NO: 1). The linker may be an NPEG linker which may be conjugated to a CPP peptide. The CPP is either Tat (Sequence: YGRKKRRQRRR; 1-letter amino acid code; SEQ ID NO: 7), as in Tat-NPEG₄(HWLKV)₂(HWLKV: SEQ ID NO: 1) (a), or Retroinverso-D-Tat (Sequence: rrrqrrkkr; 1-letter D-amino acid code; SEQ ID NO: 10), as in Retroinverso-D-Tat-NPEG₄(HWLKV)₂ (b).

In one embodiment, the said compound is selected from the group consisting of:

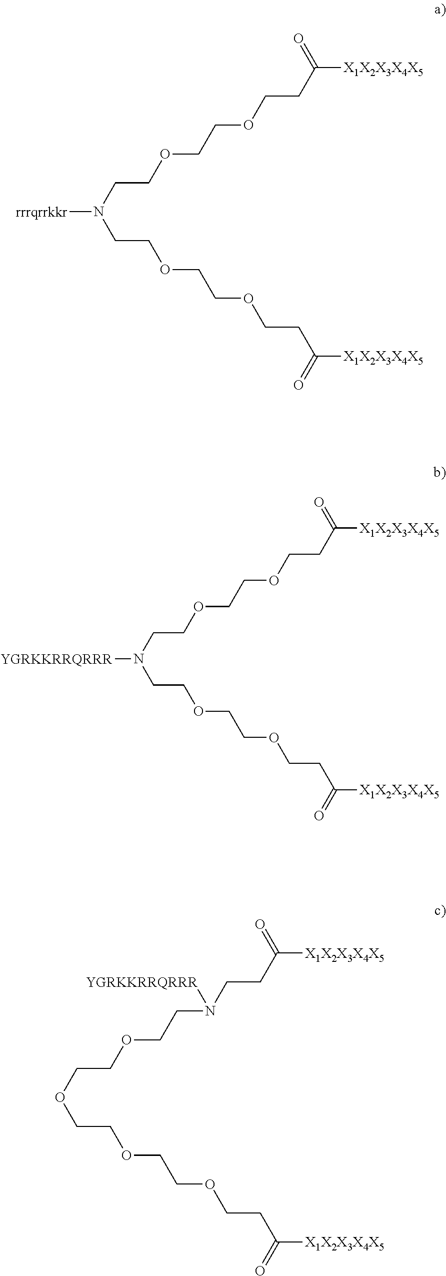

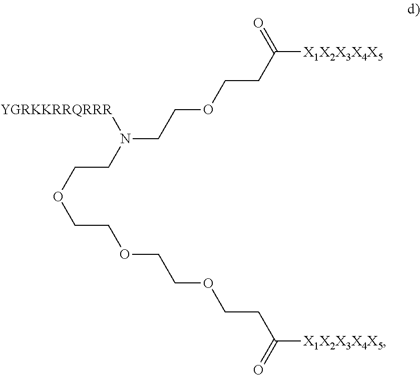

wherein X₁-X₅ is SEQ ID NOs: 2, 3, 4, 5, 6.

In one embodiment, the said compound is selected from the group consisting of:

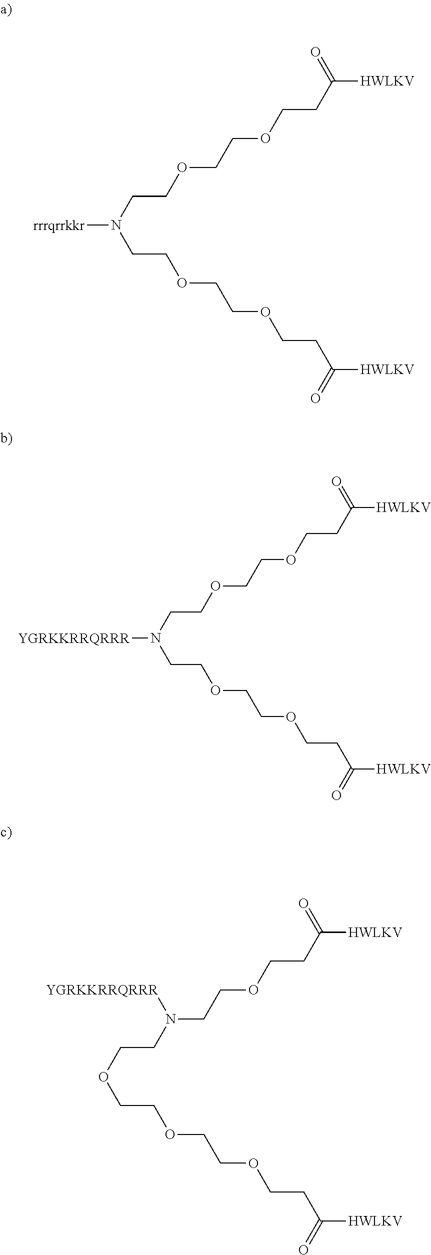

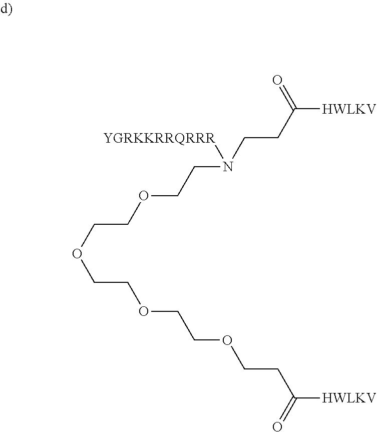

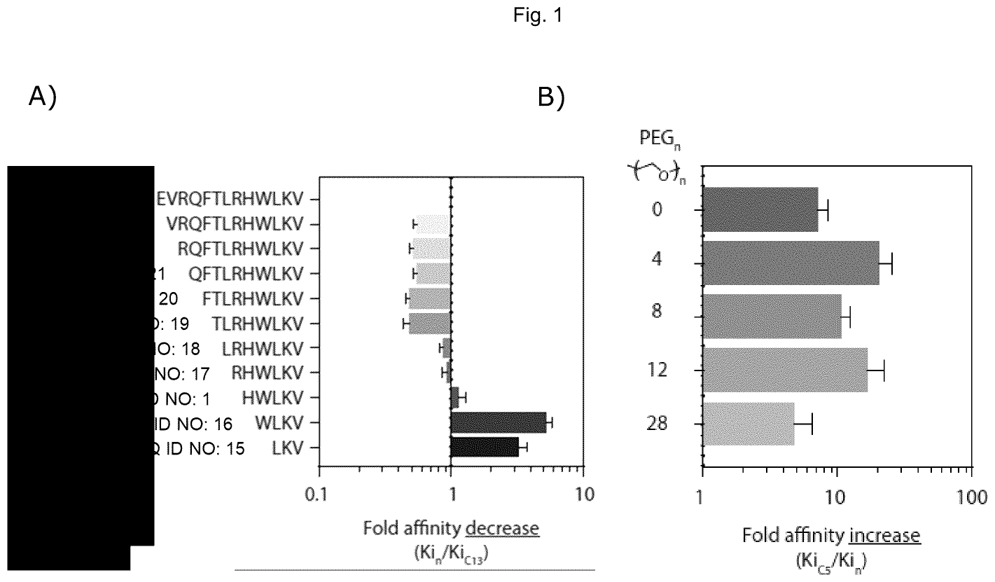

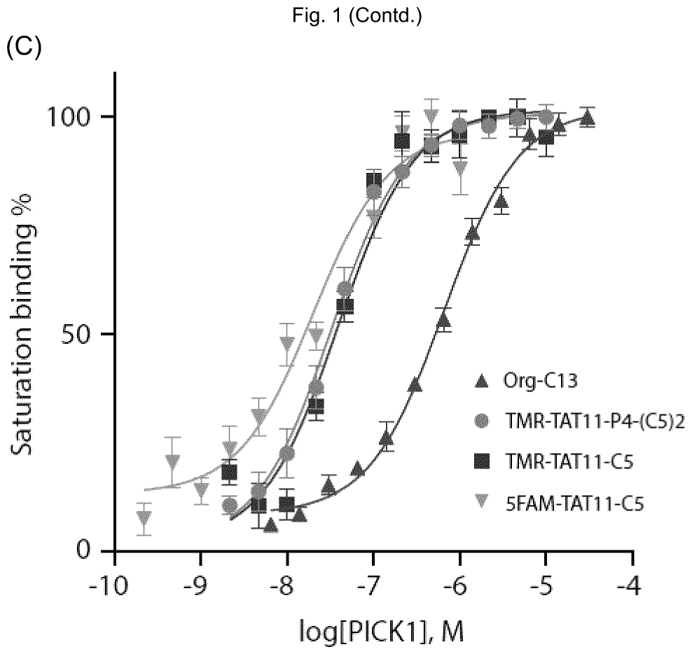

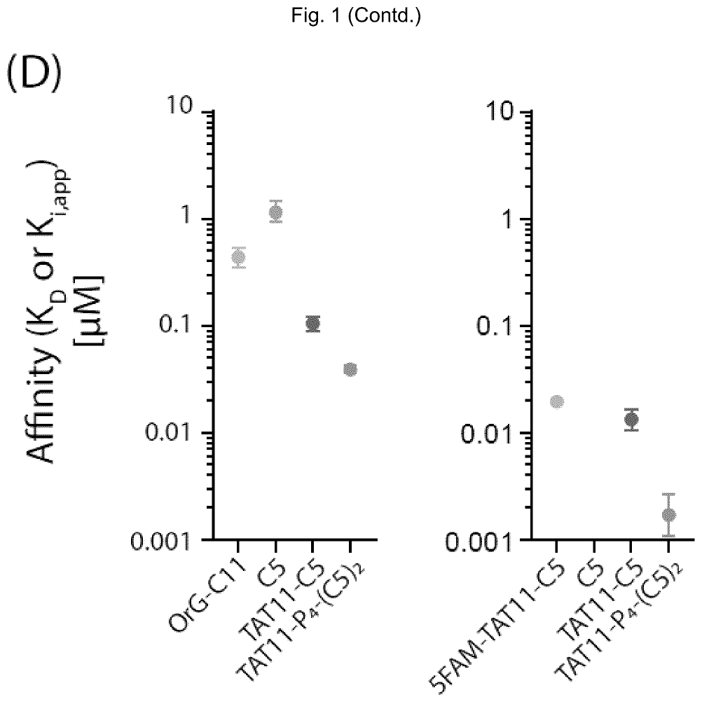

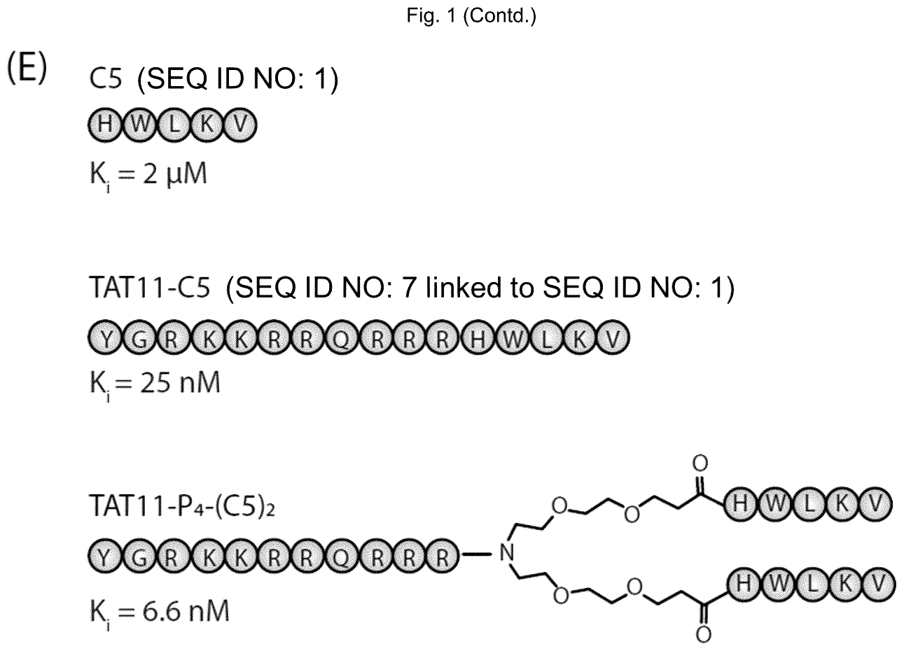

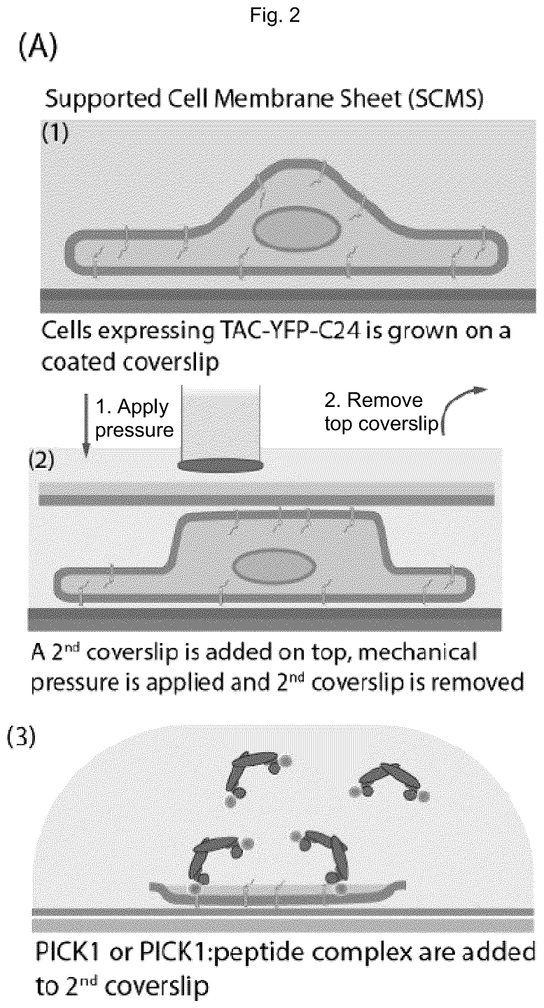

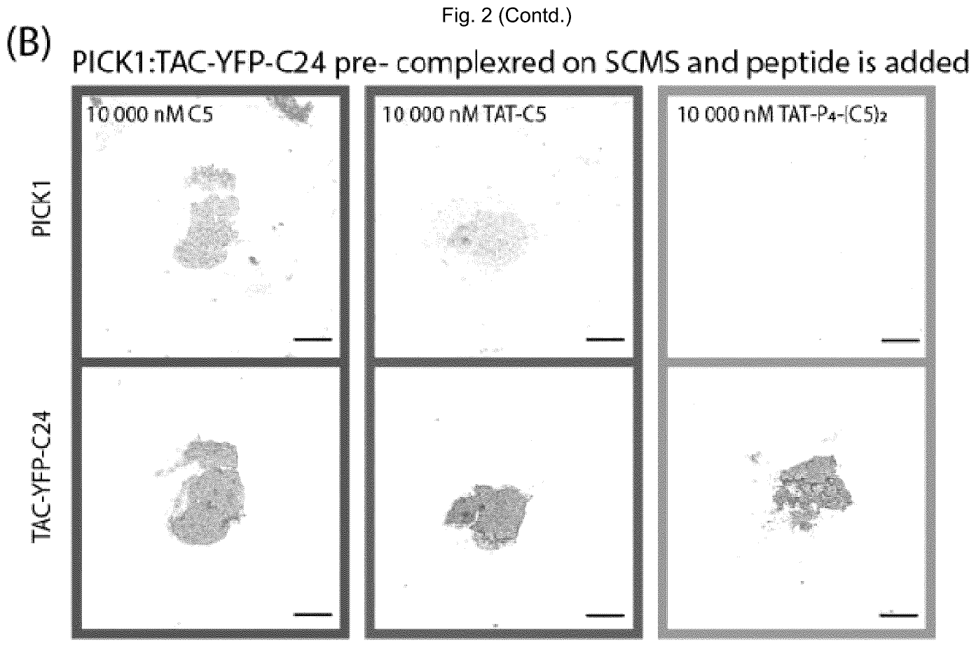

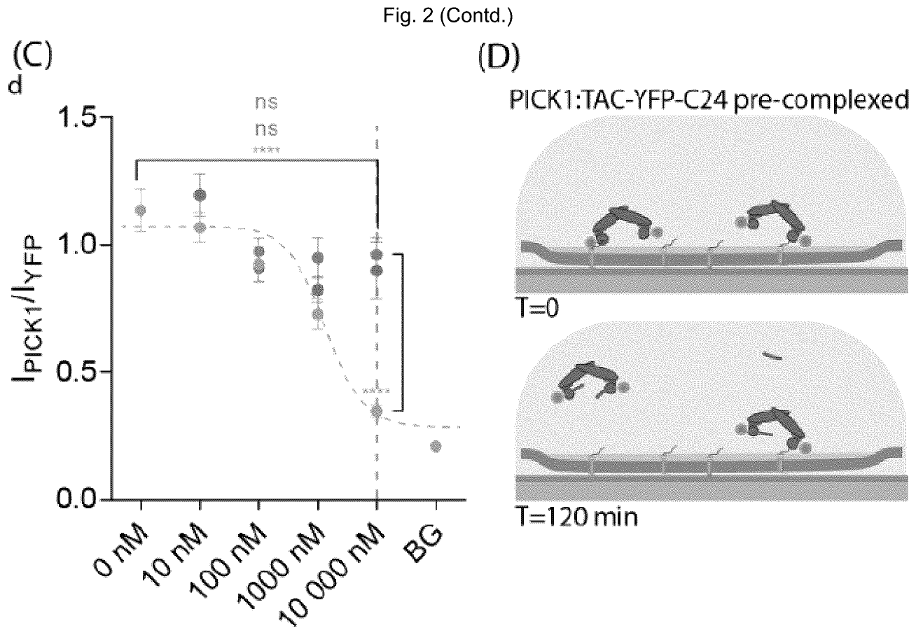

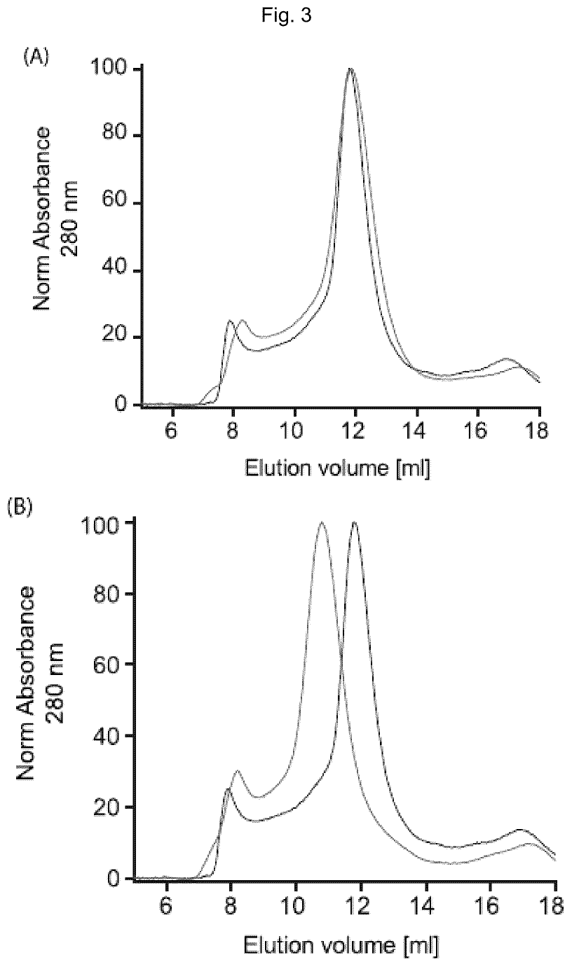

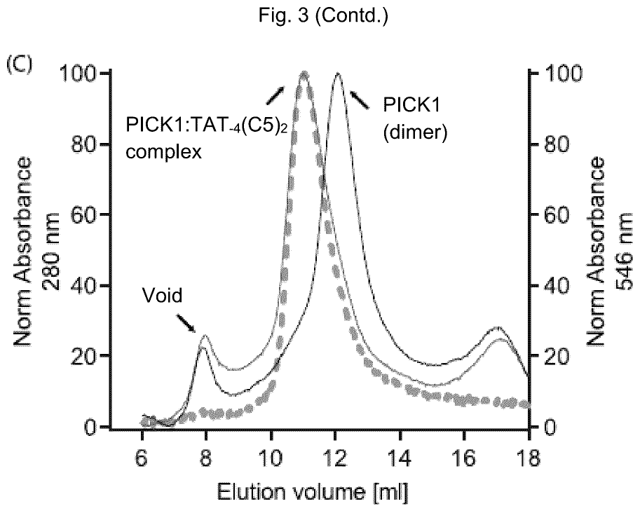

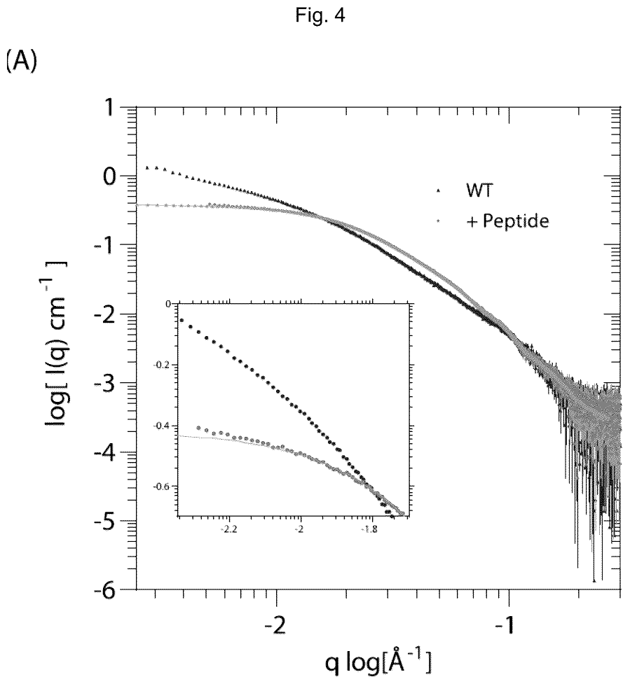

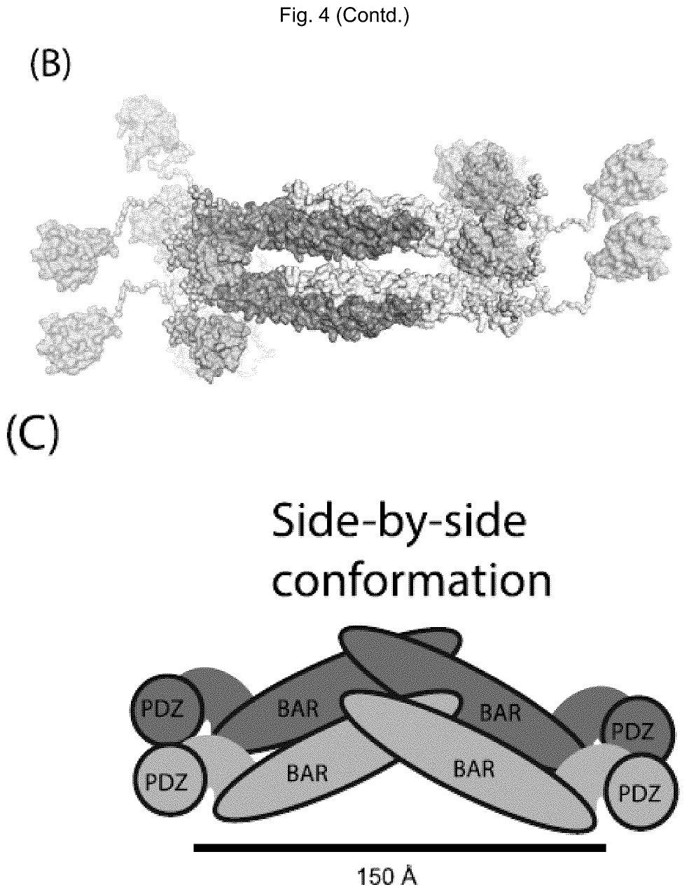

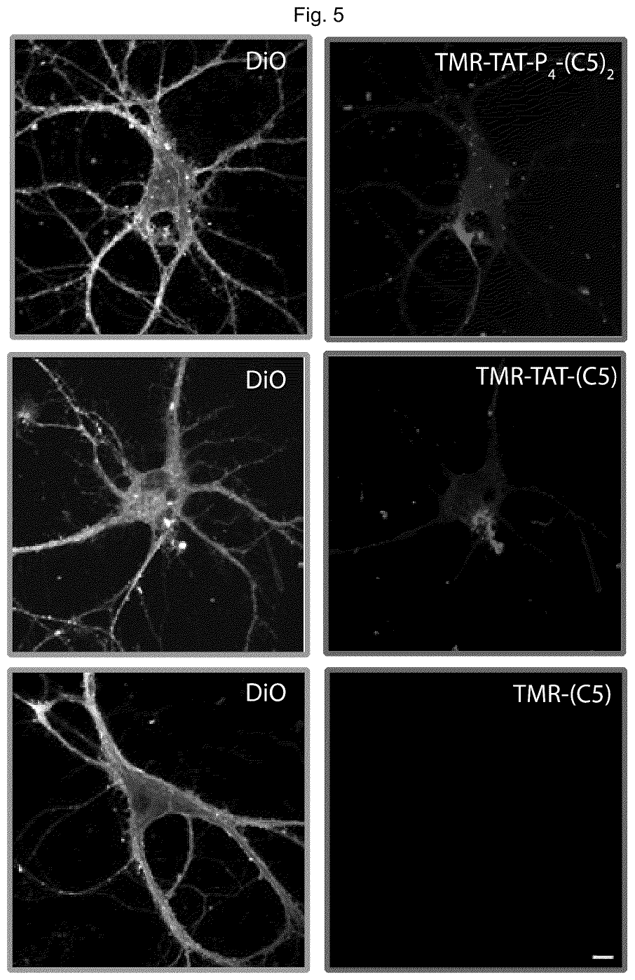

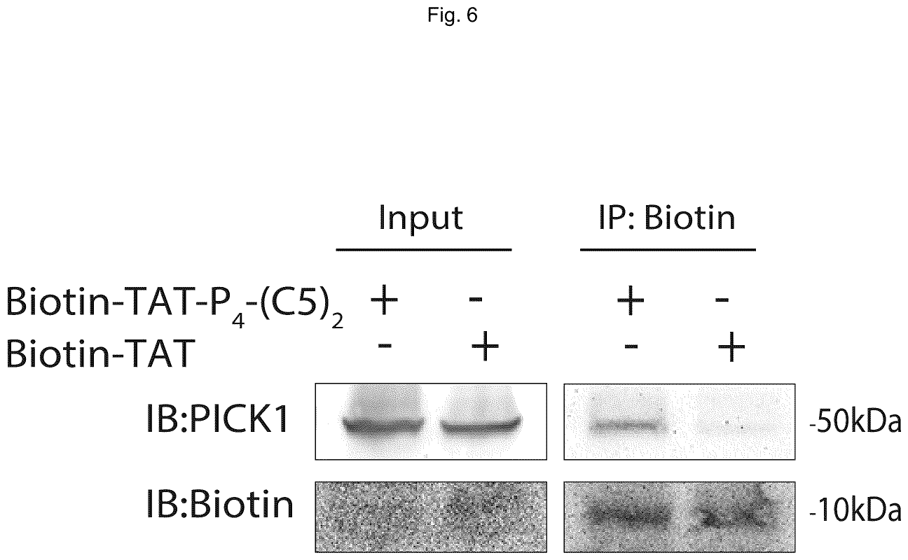

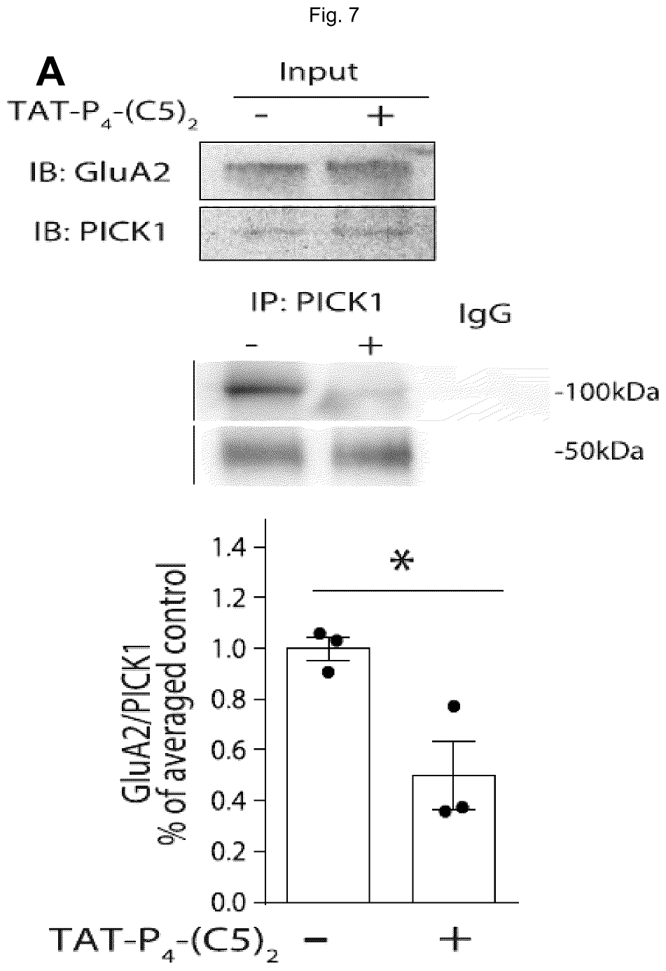

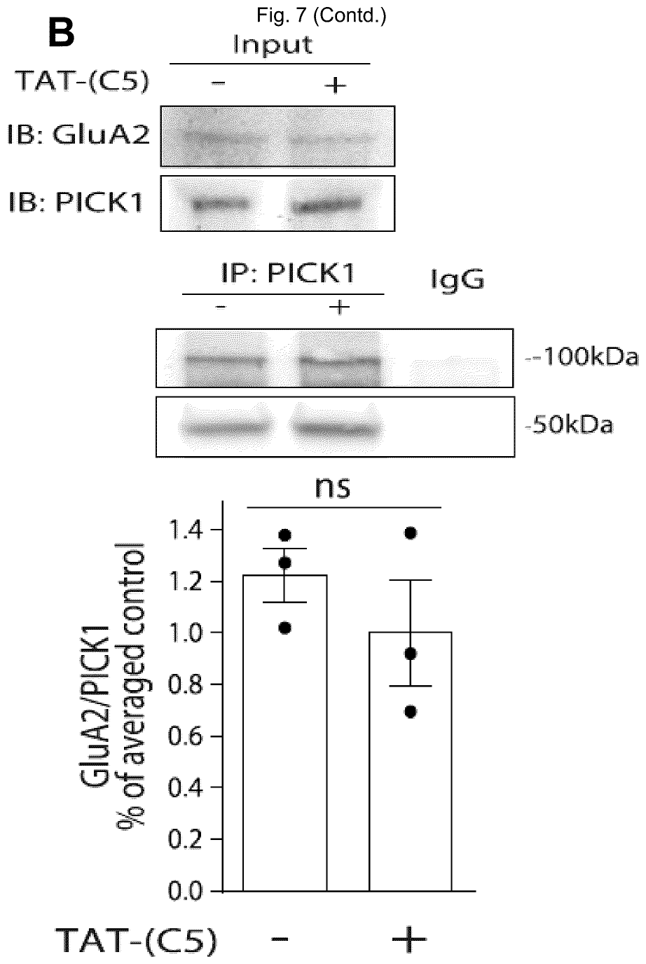

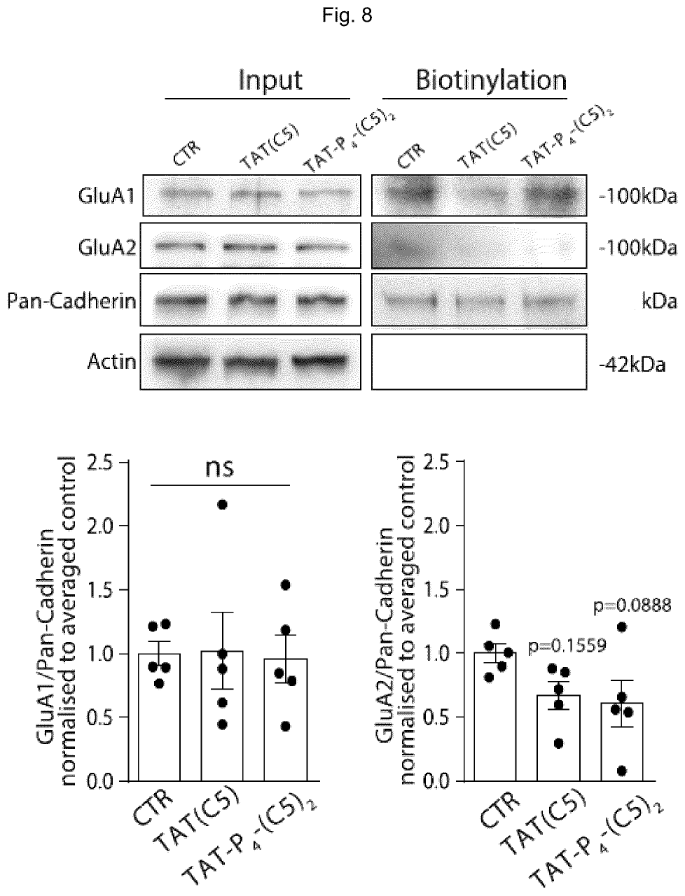

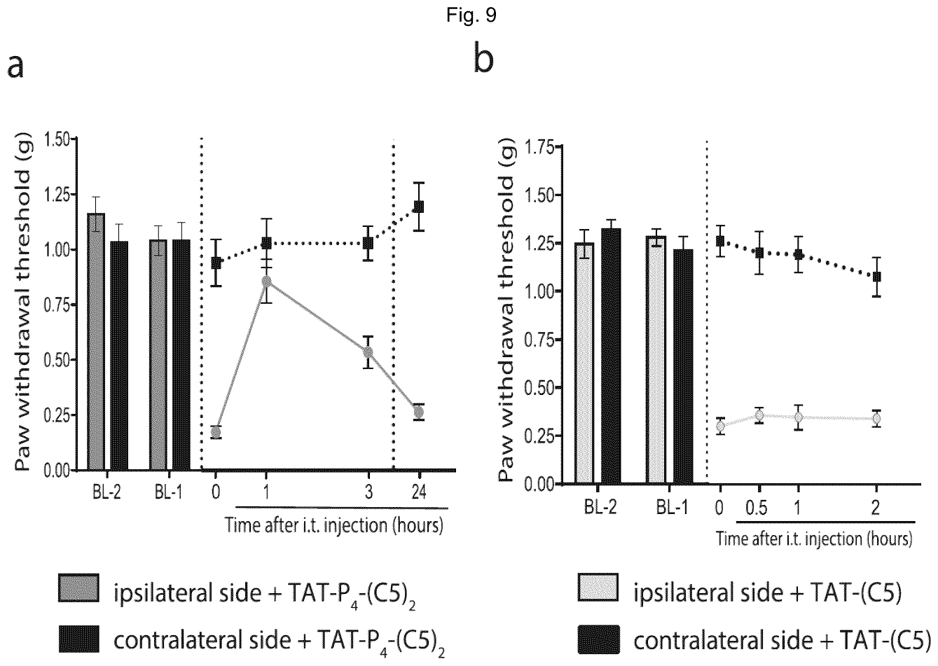

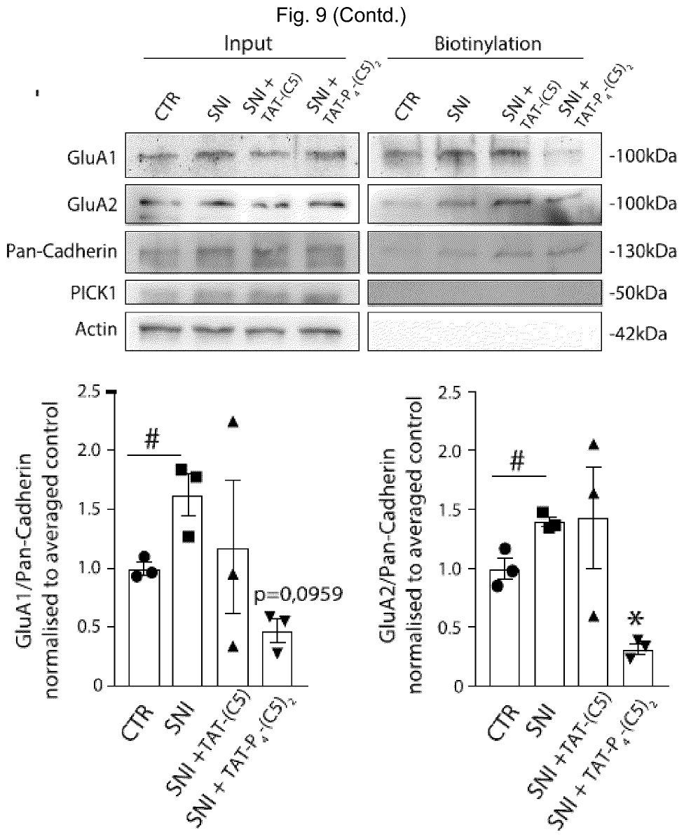

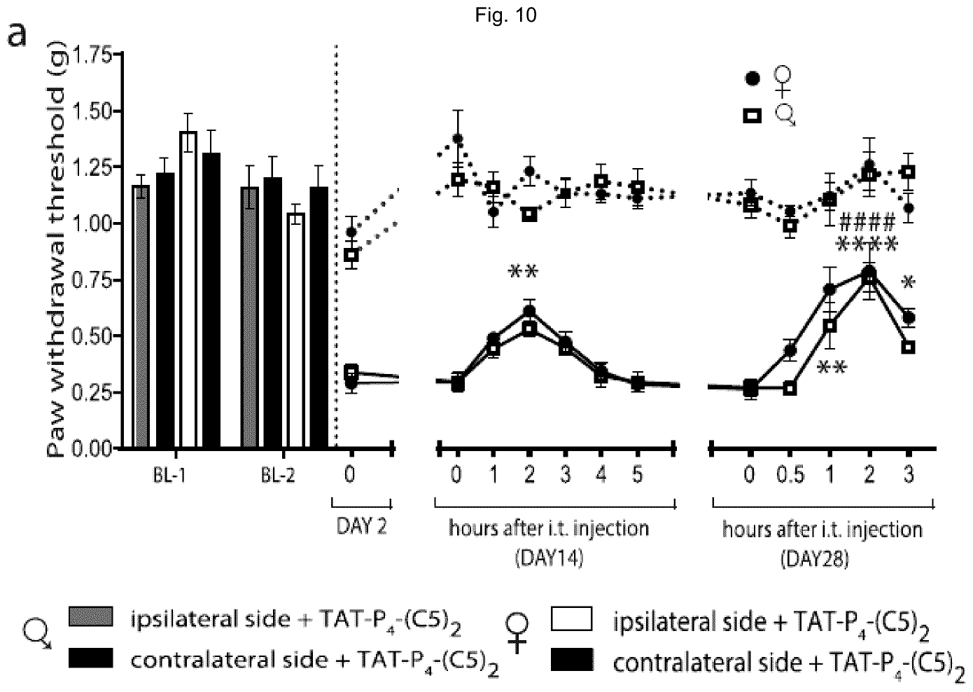

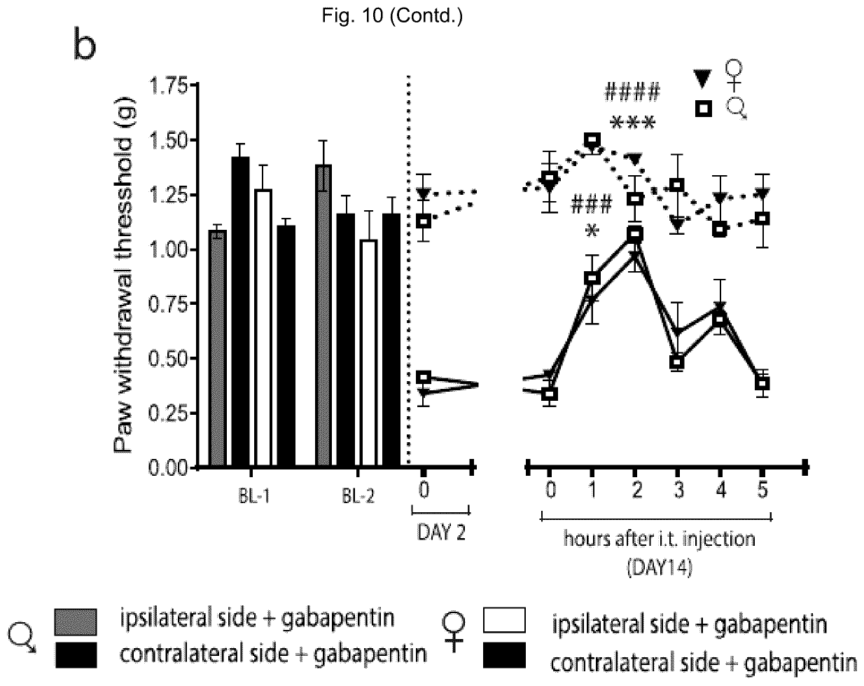

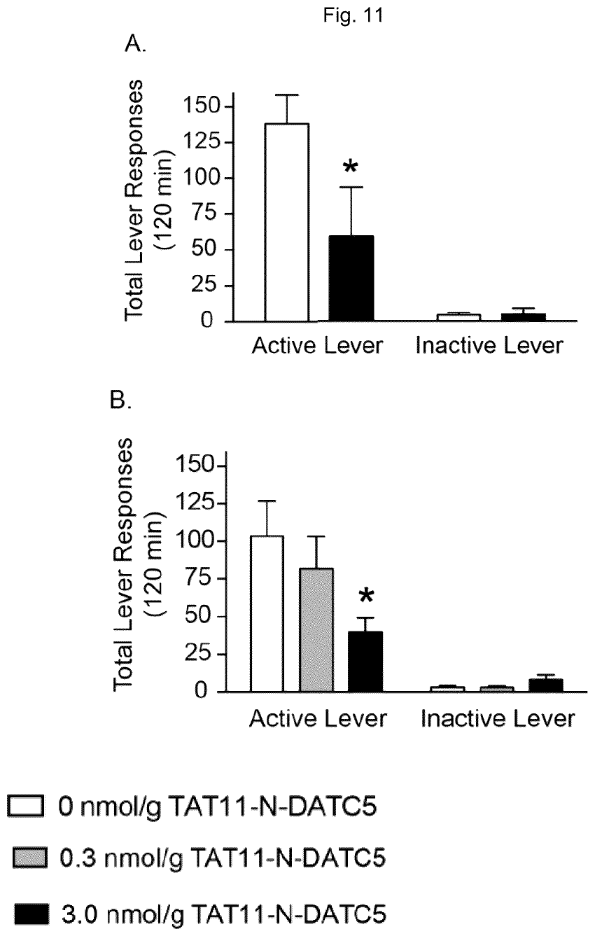

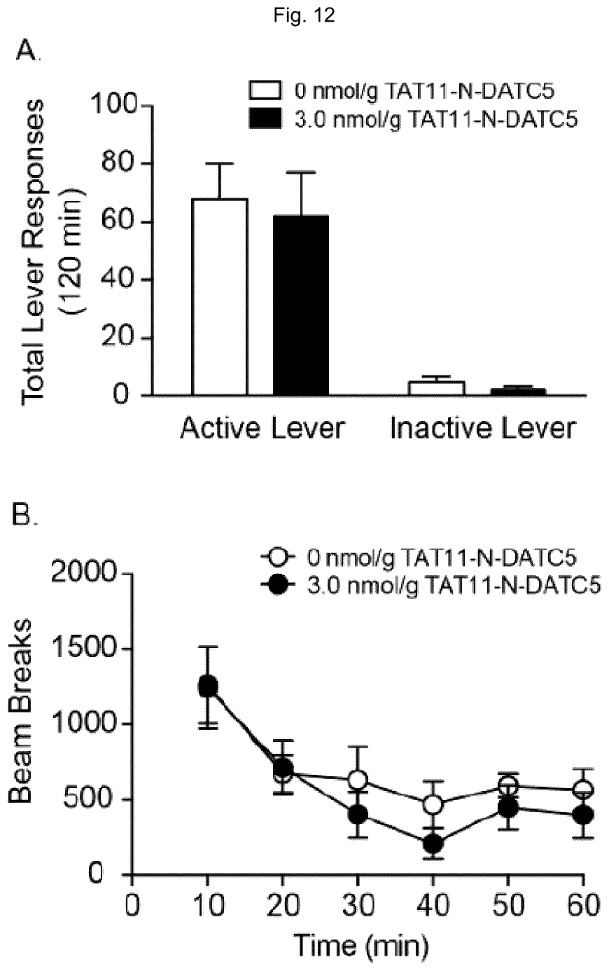

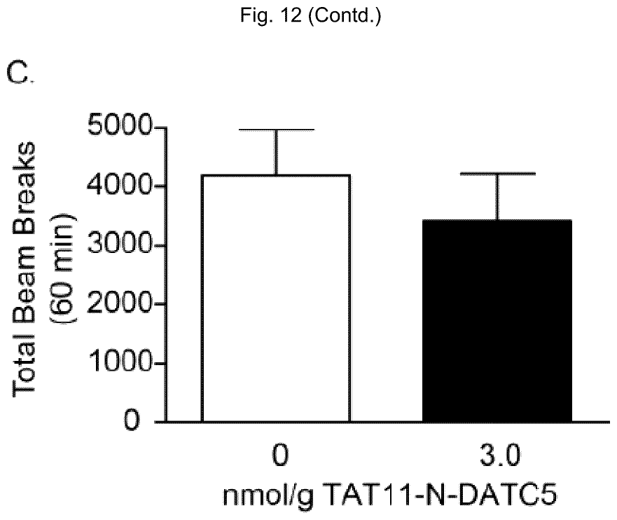

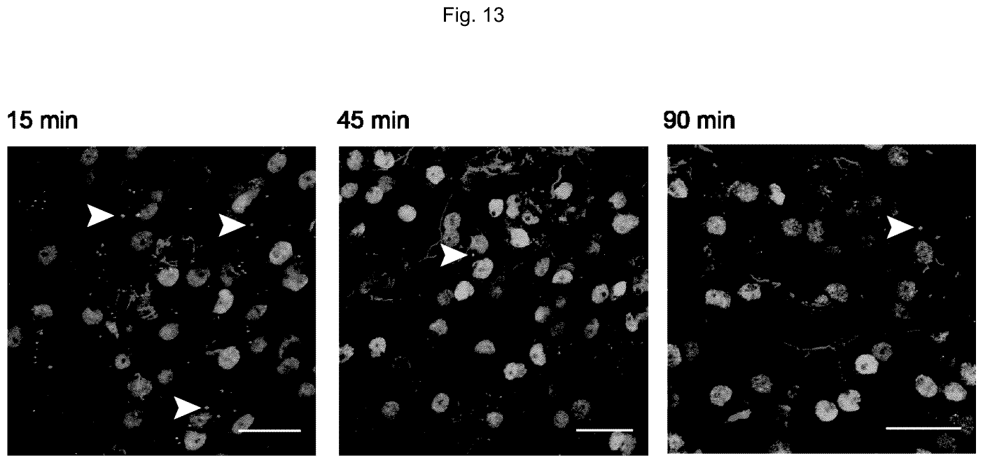

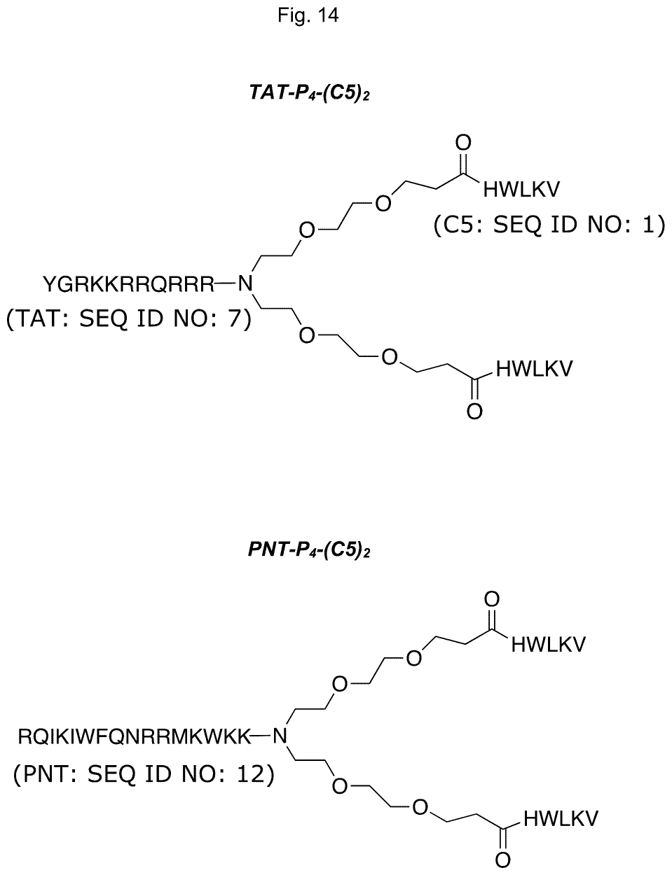

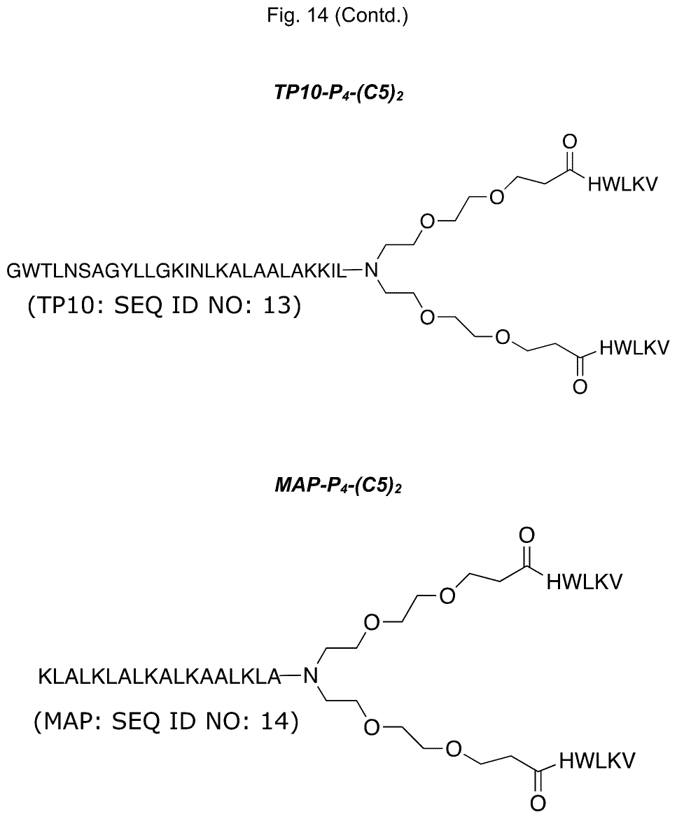

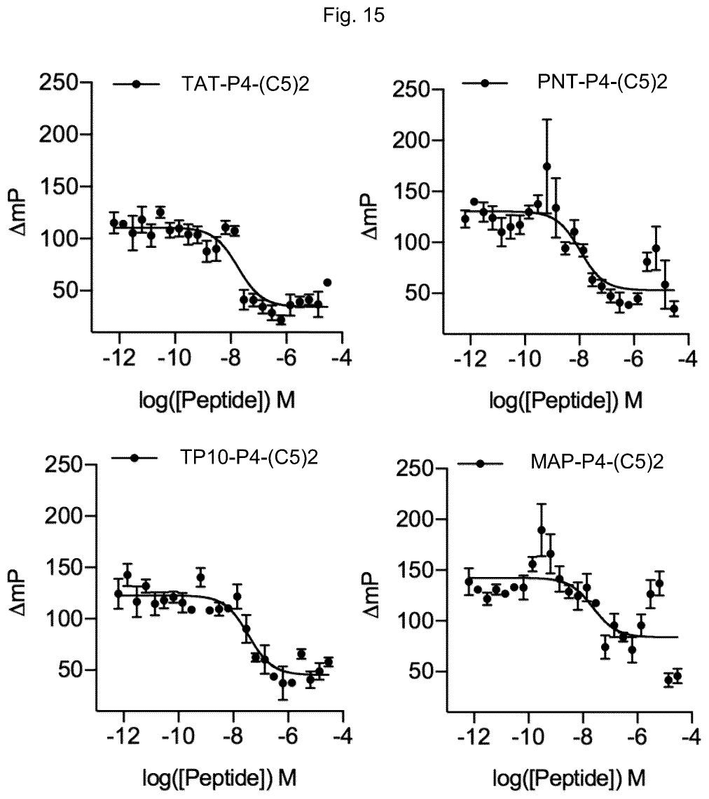

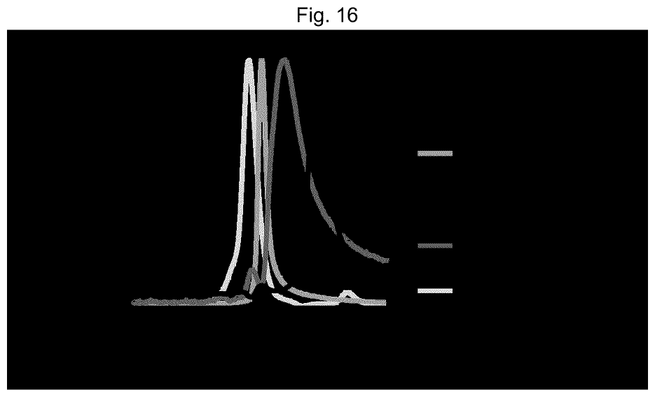

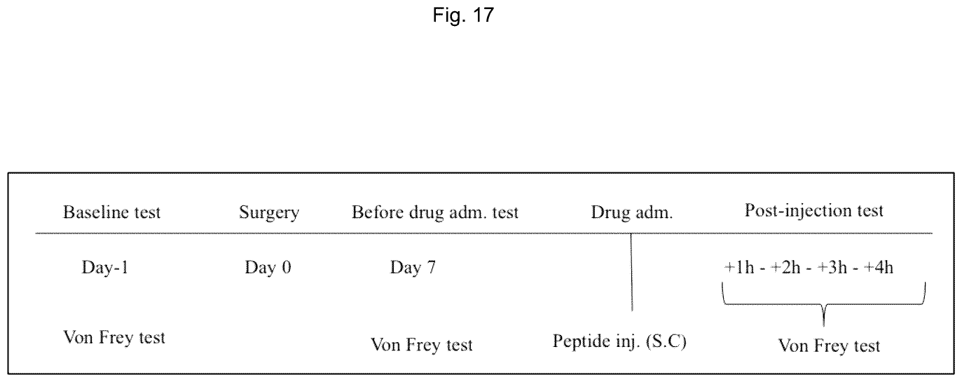

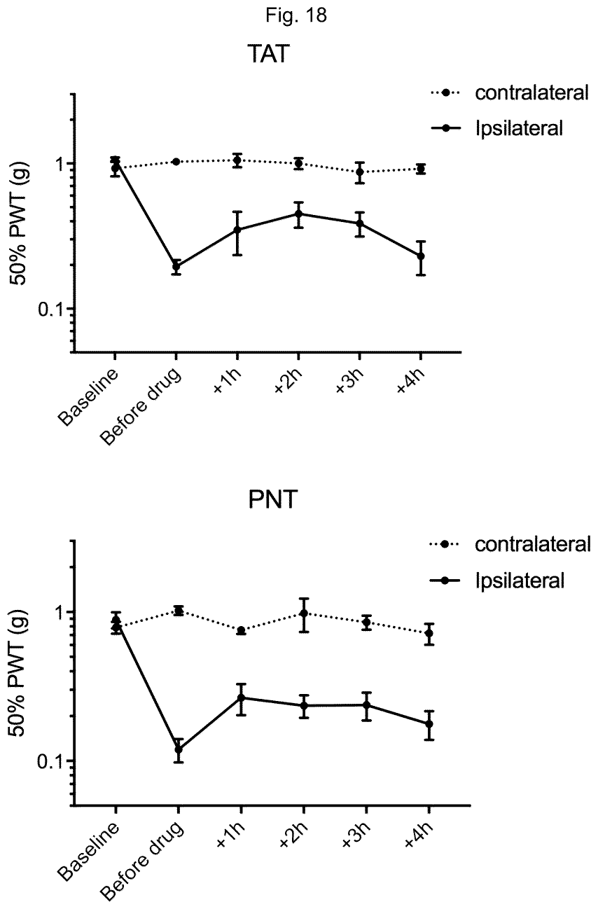

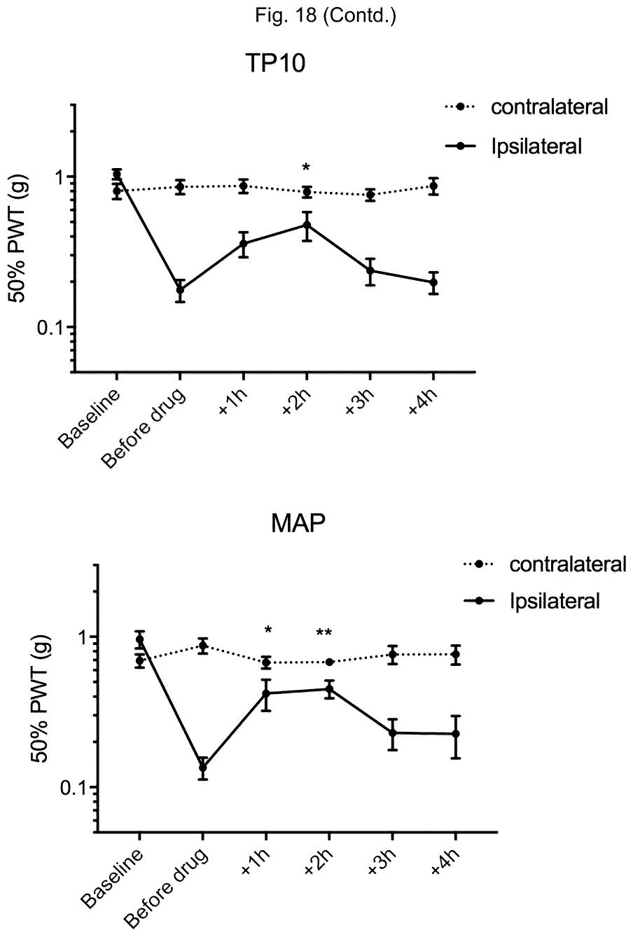

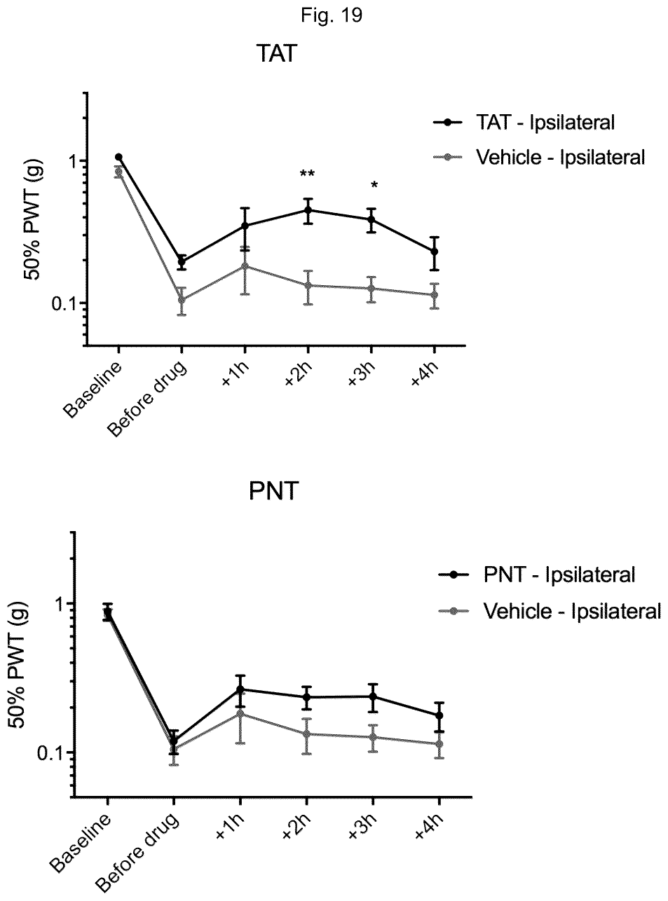

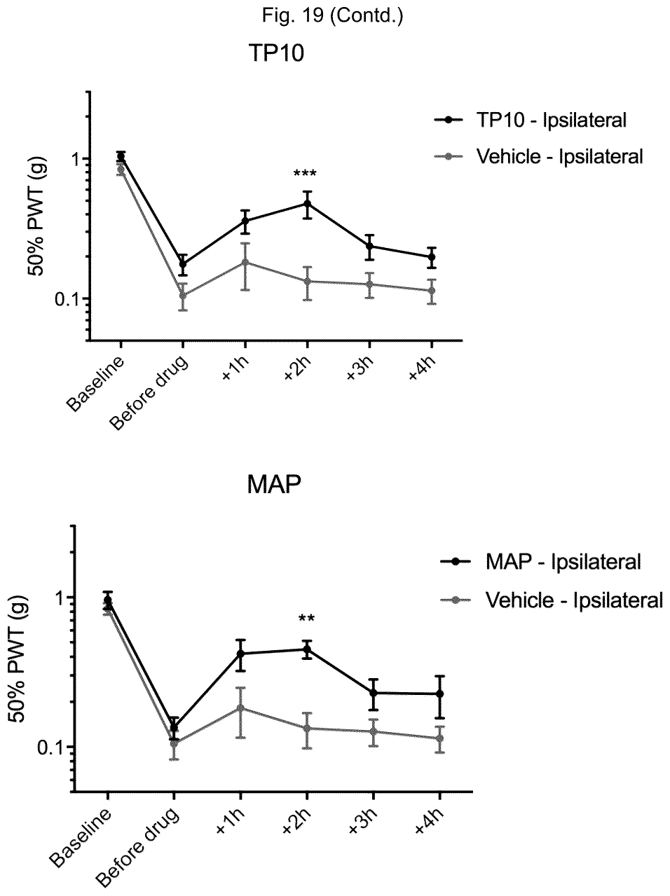

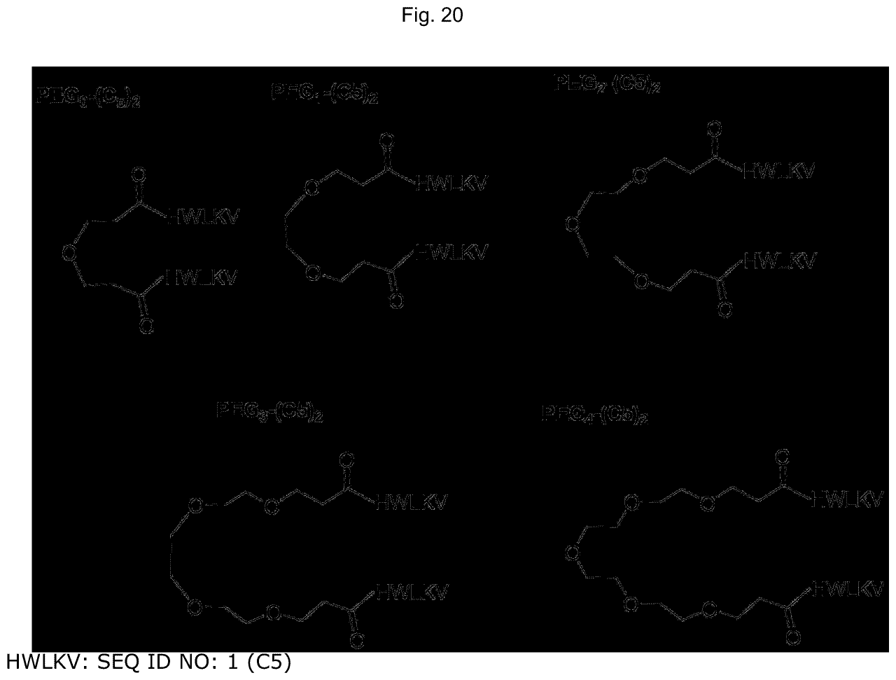

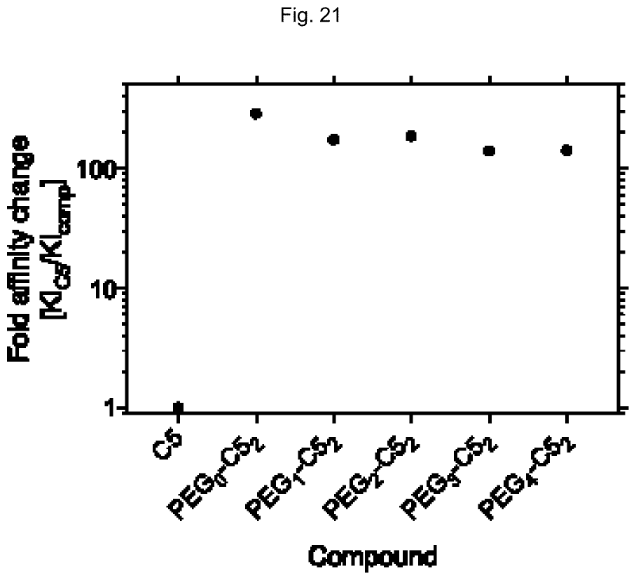

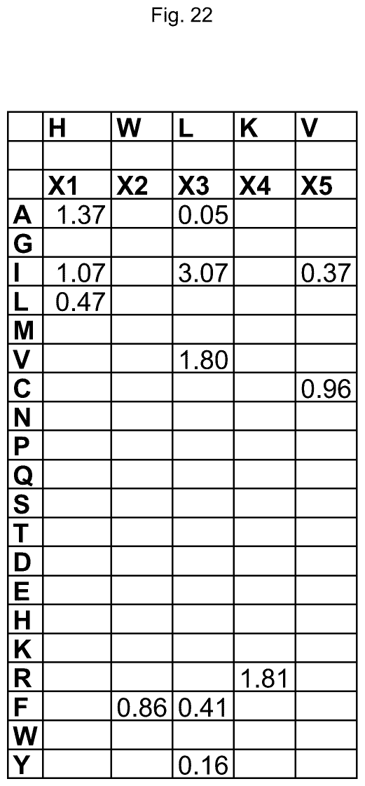

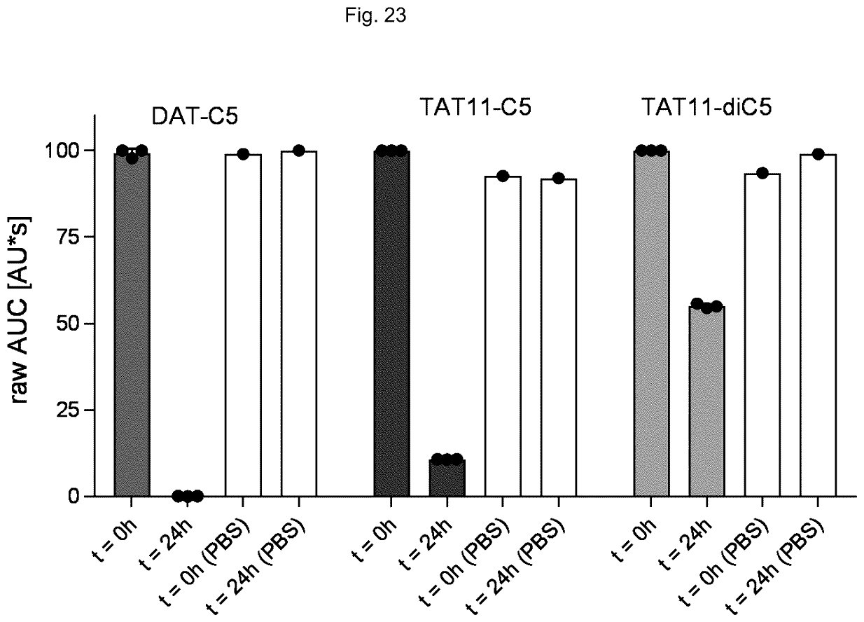

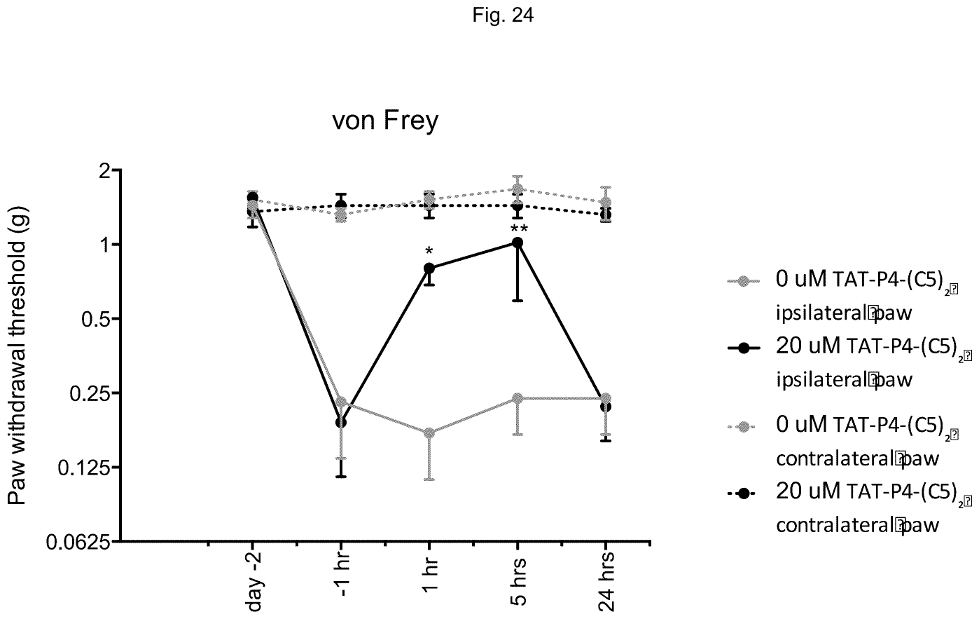

All of the dimeric PICK1 inhibitors of the present invention have an affinity for the PDZ domain of PICK1 in the nanomolar range (Examples 1 and 9), making them highly potent inhibitors (FIG. 1D, 15, and table 1). A CPP, linked to the dimeric PICK1 inhibitors of the invention, is introduced in order to improve the transport of the inhibitor across the blood brain barrier. The affinity of the dimeric PICK1 inhibitor for the PDZ of PICK1 is a critical factor in reducing the threshold concentration of drug needed to attain a therapeutic effect, which is particularly important when the drug must cross the blood brain barrier (BBB) to reach its target, since the BBB will tend to limit the accumulation of drug concentration at the target. Also, the dimeric inhibitors, but not the monomeric TAT11-C5 of similar affinity, can dissociate PICK1 bound to an interaction partner in expressed membrane supported cell membrane sheets (Example 2), by induction of a tetrameric complex of PICK1 (Example 3) in a non-native configuration (Example 4).

### AMPAR-PICK1 Interaction

AMPARs are usually only permeable to monovalent cations (i.e. Na+ and K+) due to presence of the GluA2 subunit in the receptor complex. A specific type of plasticity involving strong and sustained depolarization, however, results in a switch to AMPARs, excluding the GluA2 subunit, with increased conductance and Ca²⁺permeability (CP-AMPARs) in several types of synapses. Since the AMPARs are readily activated, this switch renders the synapse hypersensitive with respect to both Na+ and Ca²⁺ calcium influx stimulated by glutamate. This plasticity plays a central pathophysiological role in development of addiction, initially in midbrain dopaminergic neurons and subsequently, as the addiction process progresses, also in medium spiny neurons, where it underlies cocaine craving. A similar process is involved in the development of neuropathic pain, first in the dorsal horn and subsequently and conceivably, also in the neurons in thalamus and sensory cortex. Finally, CP-AMPARs are also expressed in hippocampal neurons after ischemia and as such the process rather appears to be a maladaptive type of plasticity in response to abnormal levels of glutamate in the synapse. Mechanistically, expression of CP-AMPARs involves an initial PICK1 dependent down-regulation of GluA2 containing AMPARs, which is mediated by the interaction between the PICK1 PDZ domain and the C-terminus of theGluA2 subunit of the AMPARs. This in turn allows for insertion of GluA2 lacking receptors in the synapse rendering the synapse Ca²⁺-permeable and hypersensitive.

In one embodiment, the compound binds to a PDZ domain. In another embodiment, the compound is capable of inhibiting the protein-protein interaction between AMPAR and PICK1 described above. This can thus prevent PICK1 from down-regulating GluA2 and prevent CP-AMPARs formation thereby preventing a maladaptive type of plasticity in response to abnormal levels of glutamate in the synapse. This in turn can prevent for example neuropathic pain and cocaine addiction.

In yet another embodiment, the compound inhibits PICK1. The inhibition has the same purpose as the compound binding to a PDZ domain namely preventing interaction with AMPA receptors.

In another embodiment, the compound brings together two separate PICK1 molecules. This dimerization leads to dramatic increase in affinity compared to endogenous peptide ligands. In a preferred embodiment, the compound brings together three separate PICK1 molecules. In one embodiment, the compound brings together four PICK1 molecules, such as five PICK1 molecules, for example six PICK1 molecules, such as seven PICK1 molecules, for example eight PICK1 molecules, such as nine PICK1 molecules.

The compound has a high affinity for PICK1 compared to endogenous peptide ligands. In one embodiment, said peptide has a Ki for PICK1 inferior to 10 nM, such as inferior to 9 nM, such as inferior to 8 nM, such as inferior to 7 nM, such as inferior to 6 nM, such as inferior to 5 nM, such as inferior to 4 nM, such as inferior to 3 nM, such as inferior to 2 nM, such as inferior to 1 nM. In one embodiment, the AMPAR is comprised in a cell.

### Detection

In one embodiment, the said peptide further comprises a detectable moiety. Conventional moieties known to those of ordinary skill in the art for detection can be used such as a fluorophore, a chromophore or an enzyme. The detectable moiety can be a fluorophore, 5, 6-carboxyltetramethylrhodamine (TAMRA) or indodicarbocyanine (Cy5). In another embodiment, the detectable moiety comprises or consists of a radioisotope. The radioisotope is selected from the group consisting of ¹²⁵I, ⁹⁹mTc, ¹¹¹ In, ⁶⁷Ga, ⁶⁸Ga, ⁷²As, ⁸⁹Zr, ¹²³I, ¹⁸F and ²⁰¹TI.

### Pharmaceutical Composition

The present invention provides a composition comprising a compound for use in the prophylaxis and/or treatment of diseases and/or disorders associated with maladaptive plasticity in a subject. The present invention further provides a pharmaceutical composition comprising a compound for use as a medicament. In one embodiment, the present disclosure provides a compound as disclosed herein for use as a medicament. The present invention further provides the compound or composition as disclosed herein for the manufacture of a medicament for the treatment of diseases and/or disorders associated with maladaptive plasticity.

In one embodiment, the composition is a pharmaceutical composition.

Such a composition typically contains the PICK1 inhibitor of the invention in a pharmaceutically accepted carrier.

### Disease and Disorders

The present invention provides a pharmaceutical composition for treatment of diseases and/or disorders associated with maladaptive plasticity.

AMPA-type glutamate receptors (AMPARs) are, in contrast to NMDA-type glutamate receptors (NMDARs), usually only permeable to monovalent cations (i.e. Na+ and K+) due to presence of GluA2 subunits in the tetrameric receptor complex. Plasticity changes in response to a strong and sustained depolarization, however, result in a switch to AMPARs with increased conductance and Ca2+ permeability (CP-AMPARs) in several types of synapses and this switch renders the synapse hypersensitive. Mechanistically, expression of CP-AMPARs involves an initial PICK1-dependent down-regulation of GluA2 containing AMPARs, which is mediated by the interaction between the PICK1 PDZ domain and the C-terminus of the GluA2 subunit of the AMPARs. This in turn allows for insertion of GluA2 lacking receptors in the synapse (Slot hypothesis) rendering the synapse Ca2+-permeable and hypersensitive.

CP-AMPARs are critically involved in the mediating craving after withdrawal from cocaine self-administration in rats (Conrad et al 2008). PICK1 has been implicated in the expression of CP-AMPAR in the VTA dopaminergic neurons in midbrain and in nucleus accumbens during development of cocaine craving (Luscher et al 2011 and Wolf et al 2010) suggesting PICK1 as a target in cocaine addiction. Indeed, i.v administration of compounds of the present disclosure reduces cocaine craving in an animal model of reinstatement to cocaine addiction (example 7).

Upregulation of AMPA-type glutamate receptors (AMPARs) in the dorsal horn (DH) neurons causes central sensitization, a specific form of synaptic plasticity in the DH sustainable for a long period of time (Woolf et al 2000 and Ji et al 2003). Moreover, both peripheral inflammatory pain and nerve injury induced pain, cause upregulation of Ca2+-permeable AMPARs (CP-AMPARs) (Vikman et al 2008, Gangadharan et al 2011 and Chen et al 2013). Initial evidence for a role of PICK1 in neuropathic pain came from Garry et al 2003 demonstrating that peptide inhibitors of PICK1 alleviated pain induced by chronic constriction injury (CCI). Subsequently, it was demonstrated the shRNA mediated knock down of PICK1 alleviated complete Freud's adjuvans (CFA) induced inflammatory pain and it was found that PICK1 knock-out mice completely fail to develop pain in response to spinal nerve ligation (SNL) (Wang et al 2011 and Atianjoh et al 2010). Indeed, i.t. administration of the compounds of the present disclosure reduces mechanical allodynia in a model of neuropathic pain (SNI model—example 6) and inflammatory pain (CFA model—example 13).

Both TDP-43 pathology and failure of RNA editing of the AMPA receptor subunit GluA2, are etiology-linked molecular abnormalities that concomitantly occur in the motor neurons of the majority of patients with amyotrophic lateral sclerosis (ALS). Pain symptoms in a mouse model with conditional knock-out of the RNA editing enzyme adenosine deaminase acting on RNA 2 (ADAR2) are relieved by the AMPAR antagonist perampanel, suggesting a likely symptomatic relief by the compounds of the present disclosure.

Given the effect of the compounds of the present disclosure on pain and addiction, it is reasonable to expect also good efficacy on patient with comorbidity e.g. pain patients also suffering from addiction.

Similar central sensitization is thought to underlie the allodynia in hyperalgesic priming, which serves as an experimental model for lower back pain and migraine (Kandasamy et al 2015).

Similarly, the etiology for tinnitus hold several parallels with neuropathic pain including central sensitization (Vanneste et al 2019, Peker et al 2016, and Moller et al 2007).

A role for PICK1 in the surface stabilization/insertion of CP-AMPARs has been described for oxygen-glucose depletion in cultured hippocampal neurons (Clem et al 2010 and Dixon et al 2009). This evokes PICK1 as a putative target in the protection of neural death after ischemic insult.

Loss of PICK1 has been demonstrated to protect neurons in vitro and in vivo against spine loss in response to amyloid beta (Marcotte et al 2018 and Alfonso et al 2014). Consequently, PICK1 is a putative target for symptomatic and perhaps preventive treatment of Alzheimer's disease.

PICK1 interacts and inhibits the E3 ubiquitin ligase Parkin, which is involved in mitophagy. Parkin loss of function is associated with both sporadic and familial Parkinson's disease (PD). As a result, PICK1 KO mice are resistant to 1-methyl-4-phenyl-1,2,3,6-tetrahydropyridine (MPTP)-mediated toxicity (He et al 2018). Consequently, PICK1 is a putative target for symptomatic and perhaps preventive treatment of Parkinson's disease.

Overstimulation of glutamate receptors resulting in excessive intracellular calcium concentrations is a major cause of neuronal cell death in epilepsy. The GluR2 (GluA2) hypothesis states that following a neurological insult such as an epileptic seizure, the AMPA receptor subunit GluR2 protein is downregulated. This increases the likelihood of the formation of GluR2-lacking, calcium-permeable AMPA receptor which might further enhance the toxicity of the neurotransmitter, glutamate (Lorgen et al 2017).

PICK1 is overexpressed in tumor cells as compared to adjacent normal epithelia in breast, lung, gastric, colorectal, and ovarian cancer. As judged by immunostaining breast cancer tissue microarrays, high levels of PICK1 expression correlates with shortened span of overall survival. Accordingly, transfection of MDA-MB-231 cells with PICK1 siRNA decreased cell proliferation and colony formation in vitro and inhibited tumorigenicity in nude mice (Zhang et al 2010). Consequently, PICK1 is a putative target for cancer treatment and prognostics.

In one embodiment, the compound as disclosed herein is for use in the prophylaxis and/or treatment of neuropathic pain, drug addiction, amyotrophic lateral sclerosis, epilepsy, tinnitus, migraine, ischemia, Alzheimer's disease, and/or Parkinson's disease.

In one embodiment, the compound as disclosed herein is for use in the prophylaxis and/or treatment of neuropathic pain, drug addiction, amyotrophic lateral sclerosis, epilepsy, tinnitus, and/or migraine. The drug addiction may be opioid addiction. In a preferred embodiment, the compound as disclosed herein is for use in the prophylaxis and/or treatment of neuropathic pain.

In another embodiment, the compound as disclosed herein is for use in the prophylaxis and/or treatment of pain in a subject. The pain can be inflammatory pain or neuropathic pain. The pain, to be treated, may be chronic pain, which may be chronic neuropathic pain or chronic inflammatory pain. The neuropathic pain may be induced by damage to the peripheral or central nervous system as a result of traumatic injury, surgery, or diseases such as diabetes or autoimmune disorders. The neuropathic pain may be induced by treatment with chemotherapy. Where pain persists, the condition is chronic neuropathic pain. Chronic inflammatory pain may be induced by inflammation after nerve injury, as well as being initiated by inflammation induced by alien matter, where mediators released by immune cells cause a sensitization of pain pathways, i.e. a ‘wind up’ of sensory neurons located in the spinal cord. Thus, an effective analgesic drug must be able to reach spinal cord tissue and find its target, in this case PICK1, in order to have a pain-relieving effect. Thereby, the compounds must be able to pass the blood-brain barrier and/or blood-spinal cord barrier to be able to reach spinal cord tissue.

In yet another embodiment, the compound as disclosed herein is for use in the prophylaxis and/or treatment of drug addiction. The drug addiction may be opioid addiction or cocaine addiction. For example the opioid addiction may be morphine addiction.

In yet another embodiment, the compound as disclosed herein is for use in the prophylaxis and/or treatment of head injury.

In yet another embodiment, the compound as disclosed herein is for use in the prophylaxis and/or treatment of stroke or ischemia.

In yet another embodiment, the compound as disclosed herein is for use in the prophylaxis and/or treatment of Alzheimer's disease.

In yet another embodiment, the compound as disclosed herein is for use in the prophylaxis and/or treatment of Parkinson's disease.

In yet another embodiment, the compound as disclosed herein is for use in the prophylaxis and/or treatment and/or diagnosis of cancer, such as breast cancer.

In one embodiment, the pharmaceutical composition is for use in the prophylaxis and/or treatment of neuropathic pain, drug addiction, amyotrophic lateral sclerosis, epilepsy, tinnitus, migraine, ischemia, Alzheimer's disease, and/or Parkinson's disease. In one embodiment, the pharmaceutical composition is for use in the prophylaxis and/or treatment of neuropathic pain, drug addiction, amyotrophic lateral sclerosis, epilepsy, tinnitus and migraine. The drug addiction may be opioid addiction. In a preferred embodiment, the pharmaceutical composition is for use in the prophylaxis and/or treatment of neuropathic pain. In another embodiment, the pharmaceutical composition is for use in the prophylaxis and/or treatment of pain in a subject. The pain can be inflammatory pain or neuropathic pain. The pain, to be treated, may be chronic pain, which may be chronic neuropathic pain or chronic inflammatory pain. The neuropathic pain may be induced by damage to the peripheral or central nervous system as a result of traumatic injury, surgery, or diseases such as diabetes or autoimmune disorders. The neuropathic pain may be induced by treatment with chemotherapy. Where pain persists the condition is chronic neuropathic pain. Chronic inflammatory pain may be induced by inflammation after nerve injury, as well as being initiated by inflammation induced by alien matter, where mediators released by immune cells cause a sensitization of pain pathways, i.e. a ‘wind up’ of sensory neurons located in the spinal cord. Thus, an effective analgesic drug must be able to reach spinal cord tissue and find its target, in this case PICK1, in order to have a pain-relieving effect. Thereby, the compounds must be able to pass the blood-brain barrier and/or blood-spinal cord barrier to be able to reach spinal cord tissue.

In yet another embodiment, the pharmaceutical composition is for use in the prophylaxis and/or treatment of drug addiction. The drug addiction may be opioid addiction or cocaine addiction. For example the opioid addiction may be morphine addiction.

In yet another embodiment, the composition as disclosed herein is for use in the prophylaxis and/or treatment of head injury.

In yet another embodiment, the composition as disclosed herein is for use in the prophylaxis and/or treatment of stroke or ischemia.

In yet another embodiment, the composition as disclosed herein is for use in the prophylaxis and/or treatment of Alzheimer's disease.

In yet another embodiment, the composition as disclosed herein is for use in the prophylaxis and/or treatment of Parkinson's disease.

In yet another embodiment, the composition as disclosed herein is for use in the prophylaxis and/or treatment and/or diagnosis of cancer, such as breast cancer.

In neuronal synapses, the C-termini of the GluA2 subunit of AMPA receptor subunits interact with PDZ domains of PICK1.

Thus a CPP-containing dimeric PICK1 inhibitor of the invention acts as a neuroprotectant of one or more cells or tissues providing a specific strategy for treating disease and/or disorders associated with maladaptive plasticity.

Subjects at risk or presently suffering from the above disorders and diseases may be given either prophylactic treatment to reduce the risk of the disorder or disease onset or therapeutic treatment following the disorder or disease onset. The subject may be a mammalian or human patient.

### Administration

Conventional methods, known to those of ordinary skill in the art of medicine, can be used to administer compositions to the subject or patient.

Conventional methods, known to those of ordinary skill in the art of medicine, can be used to administer the pharmaceutical formulation(s) of the present invention to the patient. The pharmaceutical compositions of the present invention can be administered alone, or in combination with other therapeutic agents or interventions. Specifically, the compositions of the present invention may further comprise a plurality of agents of the present invention. The present invention further includes a method of providing prophylaxis and/or treatment of diseases and/or disorders associated with maladaptive plasticity or pain in a subject, comprising administering the above pharmaceutical composition to the subject in need thereof.

### Diagnosis

The compounds of the present disclosure may comprise a detectable moiety. Such compounds may thus be used for diagnosis, such as by detecting PICK1 in a tissue or a sample.

Thus, the present disclosure provides a compound as disclosed herein for use in diagnosis of a disease or disorder associated with maladaptive plasticity.

In one embodiment, the compound as disclosed herein is for use in diagnosis of a disease or disorder associated with maladaptive plasticity is cancer, such as breast cancer. In one embodiment, the breast cancer is selected from histological grade, lymph node metastasis, Her-2/neu-positivity, and triple-negative basal-like breast cancer.

The present disclosure further provides a method of diagnosing breast cancer in a subject in need thereof, the method comprising the steps of:
a. obtaining a tissue sample from said subject;
b. staining the sample with the compound as disclosed herein;
c. determining the level of PICK1 in the sample; and
d. comparing the level of PICK1 in the sample to a healthy standard,

wherein an increased level of PICK1 in the sample is indicative of said individual having breast cancer.

The present disclosure further provides a method for predicting the prognosis for a subject suffering from breast cancer, the method comprising the steps of:
a. obtaining a tissue sample from said subject;
b. staining the sample with the compound as disclosed herein;
c. determining the level of PICK1 in the sample; and
d. comparing the level of PICK1 in the sample to a healthy standard,

wherein an increased level of PICK1 in the sample is indicative of poor prognosis.

In one embodiment, the compound as disclosed herein is used in stratification of subjects suffering from a disease associated with maladaptive plasticity into responders and non-responders of treatment with said compound. Such stratification may be used for assessing efficacy of the compound having a bivalent interaction with PICK1 prior to initializing other methods of treatment, such as AAV based therapies resulting in similar mechanisms of treatment, such as PICK1 inhibition. Advantages of such stratification include that only responders to the mechanism of treatment, such as PICK1 inhibition, will receive the long-lasting irreversible treatment of AAV based therapies. AAV based therapies are described in co-pending application (PCT/EP2019/?????) claiming priority from EP18201742.6 having the filing data of 22 Oct. 2018.

Thus in one embodiment, the compound of the present disclosure is used for stratifying patients with a disease and/or disorder associated with maladaptive plasticity into predictable treatment responders of the gene therapy.

In one embodiment, the compound of the present disclosure is used in stratification of a subject suffering from a disease associated with maladaptive plasticity into responders and non-responders of treatment with said compound.

### Items

1. A PICK1 inhibitor comprising:
a) a first peptide comprising an amino acid sequence of the general formula:

(SEQ ID NO: 2)
X₁X₂X₃X₄X₅;

b) a second peptide comprising an amino acid sequence of the general formula: X₁X₂X₃X₄X₅ (SEQ ID NO:2);
   wherein:
   X₁ is a proteogenic or non-proteogenic amino acid, preferably H, L, I, or A; or is absent;
   X₂ is a proteogenic or non-proteogenic amino acid, preferably W, or F; or is absent;
   X₃ is a proteogenic or non-proteogenic amino acid, preferably L, V, I, F; A, or Y;
   X₄ is a proteogenic or non-proteogenic amino acid, preferably K, or R; and
   X₅ is V, I or C; and
c) an NPEG linker linking the first peptide to the second peptide; and
d) a Cell Penetrating Peptide (CPP).

2. The PICK 1 inhibitor according to item 1, wherein the first and/or second peptide comprises an amino acid sequence of the general formula:

(SEQ ID NO: 3)
X₁X₂X₃X₄X₅:

X₁ is H, L, I, or A; or is absent;
X₂ is W, or F; or is absent;
X₃ is L, V, I, F; A, or Y;
X₄ is K, or R; and
X₅ is V, I or C.

3. The PICK 1 inhibitor according to item 1, wherein the first and/or second peptide comprises an amino acid sequence of the general formula:

(SEQ ID NO: 4)
X₁X₂X₃X₄X₅:

X₁ is H, L, I, or A;
X₂ is W, or F;
X₃ is L, V, I, F; A, or Y;
X₄ is K, or R; and
X₅ is V, I or C.

4. The PICK1 inhibitor according to item 1, wherein the first and/or second peptide comprises an amino acid sequence of the general formula: X₁X₂X₃X₄X₅ (SEQ ID NO:5):
X₁ is H, I or A;
X₂ is W or F;
X₃ is L, I or V;
X₄ is K or R; and
X₅ is V or C.

5. The PICK1 inhibitor according to item 1, wherein the first and/or second peptide comprises an amino acid sequence of the general formula:

(SEQ ID NO: 6)
X₁X₂X₃X₄X₅:

X₁ is H, or A;
X₂ is W;
X₃ is L, I or V;
X₄ is K or R; and
X₅ is V.

6. The PICK1 inhibitor according to any one of the preceding items, wherein the first and/or the second peptide comprise or consist of the amino acid sequence HWLKV (SEQ ID NO: 1).

7. The PICK1 inhibitor according any one of the preceding items, wherein the CPP is conjugated to the nitrogen atom of the NPEG-linker by an amide bond.

8. The PICK1 inhibitor according to any one of the preceding items, wherein the NPEG-linker comprises in the range of 0 to 12 ethylene glycol moieties wherein one or more of the backbone oxygen atoms is replaced with a nitrogen atom, such as in the range of 0 to 10, for example in the range of 0 to 8, such as in the range of 0 to 6, for example in the range of 0 to 4, for example in the range of 2 to 12, such as in the range of 2 to 10, for example in the range of 2 to 8, such as in the range of 2 to 6, for example in the range of 2 to 4 ethylene glycol moieties wherein one or more of the backbone oxygen atoms is replaced with a nitrogen atom, preferably the NPEG-linker comprises 4 ethylene glycol moieties wherein one or more of the backbone oxygen atoms is replaced with a nitrogen atom.

9. The PICK1 inhibitor according to any one of the preceding items, wherein the NPEG-linker has one backbone oxygen replaced with a nitrogen atom.

10. The PICK1 inhibitor according to any one of the preceding items, wherein the NPEG-linker comprises a carboxylic acid in each end.

11. The PICK1 inhibitor according to any one of the preceding items, wherein the one or more nitrogen atom of the NPEG-linker is positioned at any position along the NPEG-linker, such as for example positioned in the middle of the NPEG-linker or positioned towards one end of the NPEG-linker.

12. The PICK1 inhibitor according to any one of the preceding items, wherein the NPEG-linker is conjugated to the first and second peptide via an amide bond formed between the carboxylic acids of the NPEG-linker and the N-terminus of the first and/or second peptides.

13. The PICK1 inhibitor according to any one of the preceding items, wherein said PICK1 inhibitor has the generic structure of formula:

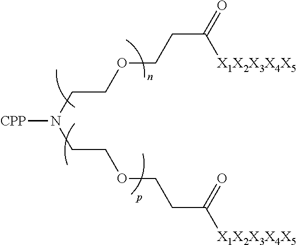

wherein
   n is an integer 2 to 12;
   p is an integer 2 to 12;
CPP is a cell penetrating peptide.

14. The PICK1 inhibitor according to any one of the preceding items, wherein said PICK1 inhibitor has the generic structure of formula:

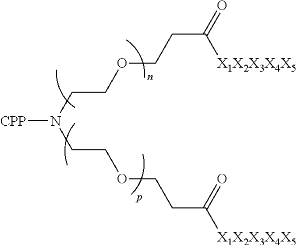

wherein
   n is an integer 0 to 12;
   p is an integer 0 to 12;
CPP is a cell penetrating peptide.

15. The PICK1 inhibitor according to any one of the preceding items, wherein the CPP comprises a TAT peptide, a Retroinverso-D-TAT peptide, a polyarginine peptide, a PNT peptide, a TP10 peptide or a MAP peptide.

16. The PICK1 inhibitor according to any one of the preceding items, wherein the CPP comprises a Tat peptide, a Retroinverso-D-Tat peptide or a polyarginine peptide.

17. The PICK1 inhibitor according to any one of the preceding items, wherein the CPP comprises a TP10 peptide or a MAP peptide

18. The PICK1 inhibitor according to any one of the preceding items, wherein the CPP is a Tat peptide comprising an amino acid sequence

YGRKKRRQRRR

(SEQ ID NO: 7) or a Retroinverso-d-Tat peptide comprising the amino acid sequence of rrrqrrkkr (SEQ ID NO: 10).

19. The PICK1 inhibitor according to any one of the preceding items, wherein said PICK1 inhibitor is selected from the group consisting of:

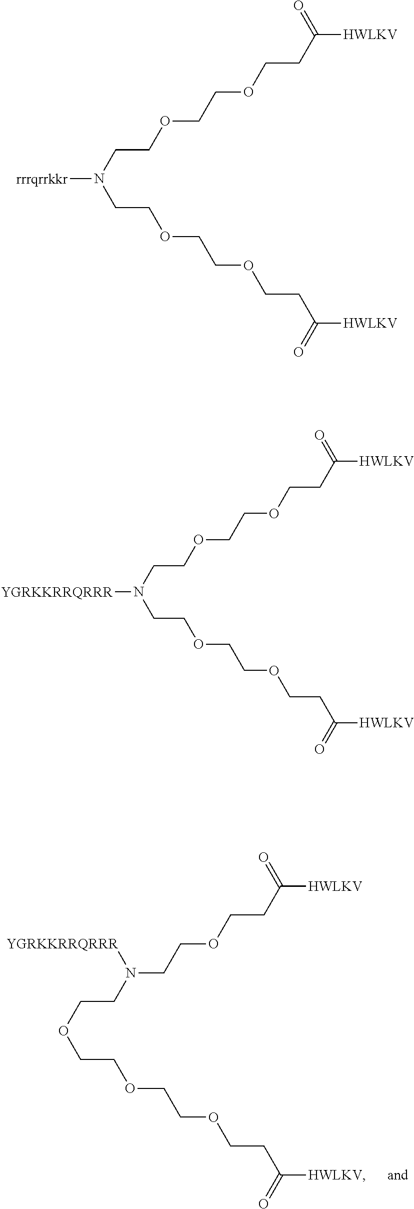

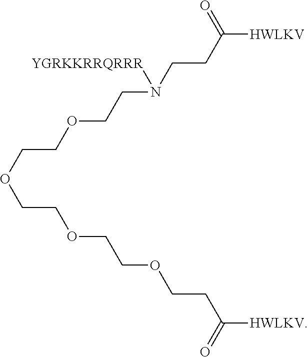

20. The PICK1 inhibitor according to any one of the preceding items, wherein the PICK1 inhibitor binds to a PDZ domain.

21. The PICK1 inhibitor according to any one of the preceding items, wherein the PICK1 inhibitor is capable of inhibiting a protein-protein interaction between AMPAR and PICK1.

22. The PICK1 inhibitor according to any one of the preceding items, wherein the PICK1 inhibitor inhibits PICK1.

23. The PICK1 inhibitor according to any one of the preceding items, wherein said peptide has a Ki for PICK1 inferior to 10 nM, such as inferior to 9 nM, such as inferior to 8 nM, such as inferior to 7 nM, such as inferior to 6 nM, such as inferior to 5 nM, such as inferior to 4 nM, such as inferior to 3 nM, such as inferior to 2 nM, such as inferior to 1 nM.

24. The PICK1 inhibitor according to any one of the preceding items, wherein the linker comprises 4 to 12 ethylene glycol moieties (x=4-12).

25. The PICK1 inhibitor according to any one of the preceding items, wherein the linker comprises 4 moieties of ethylene glycol.

26. The PICK1 inhibitor according to any one of the preceding items, wherein said PICK1 inhibitor is selected from the group consisting of Tat-PEG₄ (HWLKV)₂, Tat-NPEG₄(HWLKV)₂, TP10-NPEG₄(HWLKV)₂, and MAP-NPEG₄(HWLKV)₂.

27. The PICK1 inhibitor according to any one of the preceding items, wherein said PICK1 inhibitor is selected from the group consisting of Tat-PEG₄ (HWLKV)₂ and Tat-NPEG₄(HWLKV)₂.

28. The PICK1 inhibitor according to any one of the preceding items, wherein the peptide further comprises a detectable moiety.

29. The PICK1 inhibitor according to item 28, wherein the detectable moiety is a fluorophore, a chromophore or an enzyme.

30. The PICK1 inhibitor according to item 28, wherein the detectable moiety is 5, 6-carboxyltetramethylrhodamine (TAMRA) or indodicarbocyanine (Cy5).

31. The PICK1 inhibitor according to item 28, wherein the detectable moiety comprises or consists of a radioisotope.

32. The PICK1 inhibitor according to item 31, wherein the radioisotope is selected from the group consisting of ¹²⁵I, ^{99m}Tc, ¹¹¹In, ⁶⁷Ga, ⁶⁸Ga, ⁷²As, ⁸⁹Zr, ¹²³I, ¹⁸F and ²⁰¹TI.

33. The PICK1 inhibitor according to any one of the preceding items, for use in therapy.

34. A PICK1 inhibitor according to any one of items 1 to 32 for use as a medicament.

35. The PICK1 inhibitor according to any one of items 1 to 32 for use in the prophylaxis and/or treatment of diseases or disorders associated with maladaptive plasticity.

36. A composition comprising a PICK1 inhibitor according to any one of items 1 to 32.

37. The composition according to item 36, wherein the composition is a pharmaceutical composition.

38. A composition according to any one of items 36 to 37 for use as a medicament.

39. A PICK1 inhibitor or composition according to any one of the preceding items, for use in the prophylaxis and/or treatment of diseases or disorders associated with maladaptive plasticity.

40. The PICK1 inhibitor or composition according to any one of the preceding items for use in the prophylaxis and/or treatment of neuropathic pain, drug addiction, amyotrophic lateral sclerosis, epilepsy, tinnitus, migraine, breast cancer, ischemia, Alzheimer's disease, and/or Parkinson's disease.

41. The PICK1 inhibitor or composition according to any one of the preceding items for use in the prophylaxis and/or treatment of neuropathic pain, drug addiction, amyotrophic lateral sclerosis, epilepsy, tinnitus and/or migraine.

42. The PICK1 inhibitor or composition for use according to item 40, wherein the drug addiction is cocaine addiction.

43. The PICK1 inhibitor or composition for use according to item 40, wherein the drug addiction is opioid addiction.

44. The PICK1 inhibitor or composition for use according to item 40, wherein the drug addiction is morphine addiction.

45. The PICK1 inhibitor or composition according to any of the preceding items for use in the prophylaxis and/or treatment of pain in a subject.

46. The PICK1 inhibitor or composition according to item 45, wherein the pain is inflammatory pain or neuropathic pain.

47. The PICK1 inhibitor or composition according to any of the preceding items for use in the prophylaxis and/or treatment of cancer such as breast cancer.

48. A method of providing prophylaxis and/or treatment of diseases and/or disorders associated with maladaptive plasticity in a subject, comprising administering the PICK1 inhibitor or composition according to any one of the preceding items to the subject.

49. The method according to item 48, wherein said diseases and/or disorders associated with maladaptive plasticity is neuropathic pain, drug addiction, amyotrophic lateral sclerosis, epilepsy, tinnitus, migraine, breast cancer, ischemia, Alzheimer's disease, and/or Parkinson's disease.

50. The method according to item 48, wherein said diseases and/or disorders associated with maladaptive plasticity is neuropathic pain, drug addiction, amyotrophic lateral sclerosis, epilepsy, tinnitus and migraine.

51. Use of the PICK1 inhibitor or composition according to any of the preceding items for the manufacture of a medicament for the treatment of diseases and/or disorders associated with maladaptive plasticity.

52. The use according to item 51, wherein said diseases and/or disorders associated with maladaptive plasticity is neuropathic pain, drug addiction, amyotrophic lateral sclerosis, epilepsy, tinnitus, migraine, breast cancer, ischemia, Alzheimer's disease, and/or Parkinson's disease.

53. The use according to item 51, wherein said diseases and/or disorders associated with maladaptive plasticity is neuropathic pain, drug addiction, amyotrophic lateral sclerosis, epilepsy, tinnitus and migraine.

54. A PICK1 inhibitor according to any one of items 1 to 32, for use in diagnosis of a disease or disorder associated with maladaptive plasticity.

55. The PICK1 inhibitor for use in diagnosis according to item 54, wherein the disease or disorder associated with maladaptive plasticity is cancer, such as breast cancer.

56. The PICK1 inhibitor for use according to item 55, wherein the breast cancer is selected from histological grade, lymph node metastasis, Her-2/neu-positivity, and triple-negative basal-like breast cancer.

57. A method of diagnosing breast cancer in a subject in need thereof, the method comprising the steps of:
a. obtain a tissue sample from said subject;
b. staining the sample with the PICK1 inhibitor according to items 27-31;
c. determining the level of PICK1 in the sample; and
d. comparing the level of PICK1 in the sample to a healthy standard,
wherein an increased level of PICK1 in the sample is indicative of said individual having breast cancer.

58. A method for predicting the prognosis for a subject suffering from breast cancer, the method comprising the steps of:
a. obtain a tissue sample from said subject;
b. staining the sample with the PICK1 inhibitor according to items 27-31;
c. determining the level of PICK1 in the sample; and
d. comparing the level of PICK1 in the sample to a healthy standard,
wherein an increased level of PICK1 in the sample is indicative of poor prognosis.

59. A PICK1 inhibitor according to any one of items 1 to 32, for use in stratification of subjects suffering from a disease associated with maladaptive plasticity into responders and non-responders of treatment with said PICK1 inhibitor.

### EXAMPLES

The following examples demonstrate that the TAT-conjugated peptide is membrane permeable, that it engaged with the target protein and could interfere with PICK1-dependent phosphorylation of GluA2. Further, the bivalent high-affinity peptide, but not monovalent peptide, could actively disrupt PICK1-receptor complexes on supported cell membrane sheets, interfered with PICK1-GluA2 co-immunoprecipitation and facilitated constitutive internalization of GluA2. Furthermore, the bivalent peptide alleviated hyperalgesia for up to 4 hours in both the acute and chronic phase of the spared nerve injury model of neuropathic pain. Finally, the examples demonstrate that conjugation to a CPP provides improved plasma stability and that different CPP moieties may be used.

### Example 1

### Materials and Methods

### Protein Expression and Purification of PICK1

E. Coli cultures (BL21-DE3-pLysS) transformed with a PICK1 encoding plasmid (pET41) (Madsen et al., 2005), was inoculated in LB with kanamycin medium overnight and transferred into Luria-Betani (LB) medium with kanamycin and grown at 37° C. until OD600=0.6. Protein expression was induced with 10 mM Isopropyl β-D-1-thiogalactopyranoside (IPTG) and grown overnight at 20° C. Bacteria were harvested and resuspended in lysis buffer containing 50 mM Tris, 125 mM NaCl, 2 mM DTT, 1% TritonX-100, 20 μg/ml DNAse 1 and half a tablet of complete protease inhibitor cocktail pr. 1 L culture. Resuspended pellet was frozen at −80° C. to induce cell lysis. The bacterial suspension was thawed and cleared by centrifugation. The supernatant was collected and incubated with Glutathione-Sepharose 4B beads for 2 hrs at 4° C. under gentle rotation. The beads were pelleted at 4000×g for 5 min and supernatant was removed and beads were washed 2 times in 50 mM Tris, 125 mM NaCl, 2 mM DTT and 0.01% Triton-X100. Washed beads were transferred to a PD10 gravity column. Bead solution was incubated with thrombin protease overnight at 4° C. under gentle rotation. PICK1 was eluted on ice and absorption at 280-nm was measured on TECAN plate reader, and protein concentration was measured using lambert beers law (A=εcl), εA280PICK1=32320 (cm*mol/L)−1.

### Peptide Synthesis

Fluorescently labelled peptides were conjugated by either cysteine malamide in the case of OregonGreen peptides or N-terminal Ahx linkage in case of 5FAM labelling. PEG₀-(HWLKV)₂, PEG₄-(HWLKV)₂, PEG₈-(HWLKV)₂, PEG₁₂-(HWLKV)₂, PEG₂₈-(HWLKV)₂ was synthesized by solid phase peptide synthesis as described in (Bach et al., 2012). TMR-TAT-P₄-(C5)₂, YGRKKRRQRRR-PEG₄-(HWLKV)₂ was synthesized as described in the shown steps, previously described for Tat-N-dimer in (Bach et al., 2012). HWLKV: SEQ ID NO: 1, YGRKKRRQRRR: SEQ ID NO: 7.

### Fluorescence Polarization in Vitro Binding to PICK1

Fluorescence polarization was carried out in saturation mode and competition mode. In brief, saturation experiments were carried out using an increasing amount of protein incubated with a fixed concentration of PICK1 and fluorescent tracer (OrG-C11, 20 nM; OrG-GluA2-C13, 20 nM; TMR-TAT-4(C5)2, 4 nM) where competition was done at a fixed concentration of protein and probe, against an increasing concentration of unlabelled peptide. The fluorescence polarization was measured directly on a Omega POLARstar plate reader using excitation filter at 488-nm and long pass emission filter at 535-nm.

### Results

### Design of a High Affinity Dimeric Inhibitor of PICK1

To identify the shortest peptide sequence with conserved affinity towards the PICK1 PDZ domain, we started from the best binder identified DAT C13 (C-terminal 11 residues of the dopamine transporter, C11). Peptides successively truncated from the N-terminus retained, or even slightly increased affinity, down to DAT-C5 (C5), while further truncation slightly reduced affinity (FIG. 1A). It was hypothesized that dimeric PICK1 could be targeted using a bivalent ligand, connected with a PEG linker. Dimeric peptides of DATC5 which were fused at the N-terminus with different lengths of PEG linkers (FIG. 1B), PEGx-(C5)₂ were therefore designed. All linkers increased affinity more than 10-fold and a linker length of 4, P4-(C5)₂ which spans ˜43 Å, was optimal (Ki,app=98 nM) demonstrating a 15 fold increase in affinity compared to monomeric C5 (Ki,app=1.42 μM) (FIG. 1B). This was somewhat surprising given the distance between the PDZ domains of the PICK1 dimer was estimated to be ˜180 Å. The longest peptide (P₂₈-(C5)₂), which spans ˜130 Å, on the other hand showed the lowest affinity (Ki, app=593 nM) presumably due to increased entropic penalty (FIG. 1B).

To render the peptide cell permeable, the PEG4 linker was modified to enable conjugation to the 11 amino acids of the HIV Tat protein known to facilitate cell penetration (YGRKKRRQRRR) (SEQ ID NO: 7). The resulting peptide TAT11-PEG₄-(DATC5)₂ (TAT-P₄-(C5)₂) as well as a simple TAT-C5 (FIG. 1C) were fluorescently labelled (5FAM or TAMRA) and saturation binding curves compared to 5FAM-C5 are shown in FIG. 1D demonstrating low nanomolar affinity for not only the dimer peptide but surprisingly also for the two fluorescently labelled TAT-C5 peptides. Finally, to obtain actual affinities of the non-labelled TAT-P₄-(C5)₂ and TAT-C5, competition binding experiments were performed with a series of different fluorescent tracers to match ligand affinities. Increasing the tracer affinity in general resulted in an increased apparent affinity for TAT-(C5) and TAT-P₄-(C5)₂, and a perturbed affinity for C5. Using 5FAM-TAT-(C5) as the optimal tracer, both TAT-C5 (Ki=25 nM/80-fold shift) and TAT-P₄-(C5)₂ (Ki=6.6 nM/301-fold shift) had an increased affinity while C5 (Ki=2 μM nM) had a slight decreased affinity (FIG. 1E). TAT-P₄-(C5)₂ thus ranked among the most potent PDZ domain inhibitors.

### Example 2

### Materials and Methods

### HEK293-GT Cultures and Transfection

Human Embryo Kidney 293 GripTite cells (HEK293-GT) for SCMS were grown i in Dulbecco's modified Eagle's medium 1965 with fetal calf serum and pen-strep antibiotics. Cells were transfected using TAC-YFP-DATC24 DNA (pEYFP-C1 vector) and lipovectamin in opti-MEM® overnight. Cells were washed once with PBS and detached using 0.5% Trypsin with EDTA. Cells were then seeded into 6-well plates and allowed to grow and attach overnight at 37° C. in humidified 10% CO2 atmosphere.

### Supported Cell Membrane Sheets

The SCMS was prepared as described in (Perez et al., 2006). In brief, round cover glasses were plasma cleaned and coated with 0.3 mM poly-L-ornithine hydrobromide (Poly-ORN) for 30 min. The coating Poly-ORN was then removed. Seeded and transfected cells were then washed twice to allow them to swell. The swelled cells were then covered with the cover glass with the Poly-ORN coated side facing down on the cells. Dynamic pressure was manually applied to the cover glass using the piston from a 12 ml plastic syringe for a total of 1 min. The force from removing the cover glass causes some cells to rupture and leave a SCMS on the surface of the cover glass. The cover glass was covered in sheet buffer (10 mM HEPES, 120 mM KCl, 2 mM MgCl2, 0.1 mM CaCl2, and 30 mM Glucose at pH 7.35) with 1 mg/ml BSA to saturate unbound Poly-ORN for a total of 20 min on ice in the dark and then washed away. A protein solution was then added to the cover glass and left to incubate for the desired amount of time depending on the experiment. In the experiments using a premixed PICK1: peptide solution, 100 nM DY549-SNAP-PICK1 was incubated for at least 20 min with different concentrations of C5, TAT-(C5) or TAT-4(C5)₂. The protein: peptide solution was then added to the SCMS-cover glass and incubated for 2 hours. In the case of the pre-binding setup, the SCMS was incubated with 400 nM of DY549-SNAP-PICK1. Unbound protein was removed and peptide solution was added and left to compete for 2 hours. The SCMS cover glasses were then washed in sheet buffer once, and then twice in PBS. The SCMS was then fixed in 4% PFA and mounted onto object glasses.

### Results

### TAT-P4-(C5)2 Actively Dissociates PICK1 from Membrane Embedded Receptors

PICK1 serves its functional role as a scaffold protein facilitating PDZ domain dependent clustering of receptors at the plasma membrane as well as their phosphorylation by cytosolic kinase. To determine the efficacy of TAT-P₄-(C5)₂ and TAT-(C5) to interfere with PICK1 binding to membrane embedded proteins we took advantage of the supported cell membrane sheet (SCMS) approach (FIG. 2A). Using this approach, it was demonstrated that PICK1 interacted with the membrane embedded TacDAT C24 with a binding strength of ˜50 nM. First, fluorescently labelled recombinant rat PICK1 (300 nM SNAP543-PICK1) were incubated with increasing concentrations of C5, TAT-(C5) and TAT-P₄-(C5)₂ to compete with the binding to Tac-YFP-DAT C24 expressing SCMSs. The IC50 values (IC50 D5=3.78 μM, IC50 TD5=101 nM, and IC50 TPD5=7.0 nM) matched the affinities obtained by Fluorescence polarization (FP) competition binding.

Next, the ability of the compound to dissociate pre-bound PICK1 was determined. Following pre-incubation of fluorescently labelled PICK1 on the TacDAT C24 expressing SCMS, unbound PICK1 was washed away before incubation with increasing concentrations of C5, TAT-(C5) or TAT-P₄(C5)₂ (FIG. 2B-D). This demonstrated that TPD5 actively increased the macroscopic off-rate of bivalently bound PICK1 with an apparent IC50=1.17 μM, whereas neither C5 nor TAT-(C5) could significantly dissociated PICK1 from SCMSs.

### Example 3

### Material and Methods

### SAXS

### Sample Preparation

The PICK1 tetramer was prepared by incubating TAT11-PEG₄-di-DATC5 with freshly eluted PICK1, purified as described in protein purification in Example 1, Sample composition was verified by SEC (Superdex200, 10/300, 24 ml) to be homogeneous, and fraction was collected from main peak in a buffer consisting of TBS, 2 mM DTT, 0.01% TX100.The collected fractions were pooled and upconcentrated on a 10 kDa cut-off spin filter to a final concentration of 2.45 mg/ml. The dimer PICK1 samples were prepared by isolating the dimer peak by collecting fractions from a HiLoad Superdex200 PG 16/600, and concentrating the dimer fractions to 3 mg/ml using a 10 kDa cut-off spin filter. Concentration series were prepared for both PICK1 and PICK1: TAT-P₄-(C5)₂, ranging from 0.5 mg/ml to 3 mg/ml and 0.5 mg/ml to 2.45 mg/ml respectively.

### Data Collection and Processing

Experiments were conducted at 10° C., an exposure time of 45 minutes, 20 repeated measurements, and a wavelength of 1.23 Å. Detector masking was done by the onsite beam line scientist. Radial integration of samples and buffer was done automatically. The individual buffer and sample frames were subsequently looked through manually to check for radiation damage. The frames where radiation damage was observed were removed and the data averaging was done manually for those files.

### Data Analysis

For PICK1: TAT-P₄-(C5)₂, data merging was done by merging the low q-range data [0.01045-0.04036] Å-1 from the 0.5 mg/ml sample together with the high q-range [0.03237-0.2694] Å-1 data from the 2.45 mg/ml sample, with an overlap in the [0.03237-0.04036] A-1 q-range. The scattering data was analyzed using the ATSAS program package. Buffer subtraction, scaling, Guinier analysis and p(r) analysis was conducted using the inbuilt analysis tools of the ATSAS package. Rigid body models were created using DAMMIF120 and optimized using DAMMIN. 112 DAMMIN was run using 20 individual runs, slow mode (more dummy atoms), a Dmax of 232 Å, Rg of 68.6 Å, 15 harmonics, P1 symmetry, for other settings default values was used. The 20 dummy atom models were averaged using DAMAVER. The PDZ domains and BAR domains of PICK1 was fitted into the final DAMAVER mesh using PyMoL.

### EOM

For the Fixed structures of the EOM, the all atom relaxed MD model of the dimeric PICK1 BAR domain from Karlsen et al. 2015 was used as a model for the dimeric PICK1 BAR domain and the lowest energy structure of the PICK1 PDZ domain (PDB Entry: 2LUI) was used as the model of the PDZ domain. The unstructured C-terminal, N-terminal and PDZ: BAR linker was modelled using fully flexible dummy atoms and allowed for flexibility in the relative orientation of the domains. Tetrameric BAR domain models was created using PyMOL by duplicating, translating and rotating the dimeric BAR domain into both the models suggested by Karlsen et al., 2015 along with other possible conformations of the PICK1 tetramer. EOM (Ranch) was run for each model both as single pools and multipools on the merged dataset. For the dimeric model P2 symmetry was used, for tetrameric models P4 symmetry was used and for hexameric models P6 symmetry was used (in this case the symmetry is defined as the numbers of repetitions of the protein in the complex). The models were fitted with either complete asymmetry or with 10% symmetric structures (in this case the 10% symmetric structures relates to P2, P4 or P6 symmetry). For each BAR domain configuration a pool of 10.000 models was generated using 15 harmonics. EOM (Gajoe) also fitted the dataset against both single pool (10.000 models) and multipool inputs. Gajoe was run using 1000 generations in the genetic algorithm with 100 ensembles, a non-fixed ensemble size, with a maximum of 20 curves pr. Ensemble and 100 repetitions. Fits were evaluated on the basis of their χ2 value as well as the Dmax distribution and the Rg distribution.

### Results

### High Affinity of TAT-P₄-(C5)₂ Results from Complex Assembly with Tetrameric PICK1

PICK1 forms elongated oligomers in solution. The PDZ domains in the overlap between the individual dimers were predicted to be in much closer proximity than the two PDZ domains within the dimers (Karlsen et al., 2015). So to address whether TAT-P₄-(C5)₂ stabilized higher order PICK1 complexes, analytical size exclusion chromatography (SEC) was used.

PICK1 eluted with a main elution peak at ˜12 ml (FIG. 3A, black), corresponding to dimeric PICK1. The SEC profile was unchanged by incubation with Tat-C5 (FIG. 3A). Upon incubation with TAT-P₄-(C5)₂, however, the main elution peak shifted ˜1 ml towards a larger molecular weight complex, far above the hypothetical mass increase of 3 or 6 kDa resulting from binding of 1 or 2 peptides (FIG. 3B, gray). Incubation of PICK1 with the fluorescently labelled TMR-TAT-P₄-(C5)₂ peptide showed overlapping curves of the 280 absorbance and the 546 fluorescence (dashed line) confirming that the left shifted peak indeed contained both PICK1 and the peptide (FIG. 3C). In order to elucidate the number of PICK1 subunits in the complex, small angle X-ray scattering was used. The pair distance distribution, p (r), was dramatically altered by incubation of PICK1 with TAT-P₄-(C5)₂ (gray) compared to PICK1 alone (black), suggesting major conformational changes to the quaternary structure (FIG. 4A). The changes were most evident at low q (FIG. 4A, insert) indicating that large oligomers/aggregates previously observed for PICK1, were absent from the complex of PICK1: TAT-P₄-(C5)₂. Moreover, I (0) of the peptide complex averaged 3.99 across the concentration range without any concentration dependence, strongly suggesting formation of a tetrameric PICK1 complex by TAT-P₄-(C5)₂.

Since PICK1 is highly flexible and DAMMIN modelling gave poor fits as for PICK1 alone, we used ensemble optimization method (EOM) (Bernado et al, 2007; Tria et al., 2015), on a dataset merged from different concentrations, to determine the structural organization of the complex. The best fit was obtained for a configuration of a compact tetrameric state (FIG. 4B and C), where the BAR domains aligns themselves in a side-by-side conformation. This indicates that TAT-P₄-(C5)₂ induces a stable, compact, non-native tetrameric state of PICK1 presumably with bivalent peptide bridging PDZ domains from two individual PICK1 dimers.

### Example 4

### Materials and Methods

### Primary Cultures of Rat Hippocampal Neurons

Hippocampal neurons were prepared from prenatal E19 Wistar rat pups (mixed gender). Briefly, brains were isolated from 6-8 rat embryos brains were dissected in dissection medium [HBSS supplemented with 30 mM glucose, 10 mM HEPES pH=7.4, 1 mM sodium pyruvate, 100 u/mL penicillin, 100 μg/mL streptomycin and the cerebellum and meninges removed before dissection out the hippocampi from both hemispheres. The hippocampi were treated with papain for 20 minutes at 37° C., triturated using differentially fire-polished Pasteur pipettes, and filtered through a 70 μM cell strainer. Dissociated neurons were seeded at a density of 50,000-100,000 cells/coverslip on poly-L-lysine-coated 25 mm glass coverslips emerged in Neurobasal medium (supplemented with 2% (vol/vol) glutamax, 1% pen/strep, 2% (vol/vol) B27 and 4% FBS. After 24 hours, the growth medium was substituted with serum-free medium, and cells were grown for 21 DIV, with addition of fresh growth medium every 3-4 days.

### Immunocytochemistry

21 DIV hippocampal neurons were incubated with 5 μM TMR-TAT-P₄-(C5)₂ or TMR-TAT-(C5) or TMR-(C5) for 1 h in conditioned media at 37° C., rinsed and incubated with 5 μM of the membrane dye DiO for 10 minutes at room temperature. The hippocampal neurons were fixed in 4% PFA+4% sucrose and mounted on coverslips. Concerning the compound penetration at lower concentration DIV hippocampal neurons were incubated with 5 nM of TMR-TAT-P4-(C5)₂ for 1 hour at 37° C., rinsed and fixed in 4% PFA+4% sucrose for 20 minutes, rinsed, permeabilised and then blocked in 0.05% Triton-X100 and 5% goat serum for 20 minutes at room temperature. 14 DIV hippocampal neurons were then labelled with primary antibody followed by staining with goat-anti rabbit Alexa-488. After three final washes with PBS the coverslips were mounted with mounting medium.

### Imaging

All imaging was done on a Zeiss LSM 510 confocal laser-scanning microscopy.

### Results

### TAT-(C5) and TAT-P₄-(C5)₂ are Membrane Permeable and Engage with the Target

To confirm that the TAT peptide confers membrane permeability, dissociated hippocampal neurons were incubated with the rhodamine labelled TAT-P₄-(C5)₂, TAT-(C5) or (C5) together with the membrane dye DID. Both the TAT-fused peptides labelled neurons, whereas (C5) as expected did not. Inspection of the 3D profile of the somatic region further revealed that the DID staining enclosed a significant fraction of rhodamine staining of both TAT-P₄-(C5)₂ and TAT-(C5). TMR-TAT-(C5) in general, however, showed a more punctuate distribution, whereas the TMR-TAT-P₄-(C5)₂ was mostly diffuse (FIG. 5).

Knock down of PICK1 significantly reduced the amount TMR-TAT-P₄-(C5)₂ accumulated in hippocampal neurons, and target engagement was further substantiated by colocalization of TMR-TAT-P₄-(C5)₂ with GFP-PICK1 but not the PDZ deficient mutant GFP-PICK1 A87L or GFP in heterologous cells. Finally, we were able To pull down GFP-PICK1, but not GFP-PICK1 A87L using a biotinylated TAT-P₄-(C5)₂ from HEK293 cells. Together, these data strongly support in vitro target engagement. Lastly, the ability of TAT-P₄-(C5)₂ to engage the target protein in vivo, following intrathecal administration in spinal cord of naïve mice, was confirmed by pull down experiment between PICK1 protein and the N-terminally biotinylated TAT-P₄-(C5)₂ peptide (FIG. 6)

In summary, these data clearly support that both peptides are membrane permeable, and target PICK1.

### Example 5

### Materials and Methods

### Co-Immunoprecipitation

### Acute Hippocampal Slices and Lysates Preparation

Acute hippocampal brain slices were prepared from adult male C57BL/6 mice (8-16 weeks old). The hippocampi were quickly dissected and sliced into ice-cold aCSF buffer (124 mM NaCl, 3 mM KCl, 26 mM NaHCO3, 1.25 mM NaH2PO4, 1 mM MgSO4, 2 mM CaCl2 and 10 mM D-glucose) and placed in carboxygenated aCSF for 1 h recovery. Slices were then incubated with 20 μM TAT-P₄-(C5)₂ or TAT-(C5) for 1 hour followed by 20 uM NMDA for 3 minutes for chemical LTD induction, in aCSF buffer.

Hippocampal slices were lysed in lysis buffer (50 mM Tris Ph 7.4, 150 Mm NaCl, 0.1% SDS, 0.5% NaDeoxycholate, 1% Triton X-100, 5 mM NaF and 1×Roche protease inhibitor cocktail), and protein was incubated overnight at 4° C. with antibody (either anti-PICK1 or anti-GluA2). Protein G agarose bead slurry was added for 3 hours and the beads were then washed in lysis buffer. Proteins were eluted in 2 X Laemmli sample buffer before western blotting.

### Spinal Cord Lumbar Tract Total Lysates Preparation

Spinal cord lysates were prepared from 10 weeks old C57BL/6 mice. The animals were injected intrathecally with 20 μM TAT-P₄-(C5)₂ or TAT-(C5) and sacrificed 1 hour post-injection. The spinal cords were dissected in ice-cold PBS1X by hydraulic extrusion according to the procedure described in Richner et al., 2017. The lumbar tract of the spinal cords was quickly harvested and lysated in lysis buffer. The same procedures described for hippocampal slices were used to produce spinal cord lysates. Transfected HEK 293 cells total lysates preparation

The same procedures described for hippocampal slices were used to produce HEK cell lysates.

### Surface Biotinylation

Spinal cords were extruded from 8-12 week old naïve and SNI mice and lumbar tract was dissected as in Richner et al., 2011 and transverse slices were generated using a Mellwan tissue chopper. Slices were then placed and separated in ice-cold CSF (124 mM NaCl, 3 mM KCl, 26 mM NaHCO3, 1.25 mM NaH2PO4, 1 mM MgSO4, 2 mM CaCl2 and 10 mM D-glucose) before a recovery in carboxygenated aCSF for 1 hour. Peptide inhibitor incubation was then performed for 1 hour. Slices were incubated in 1 mg/ml biotin in ice-cold carboxygenated aCSF for 45 minutes, washed 3 times in 10 mM glycine, 1 time in tris-buffer-saline (TBS: 20 mM Tris, 150 mM NaCl, pH 7.6), homogenized in lysis buffer (25 mM TRIS pH 7.6, 150 mM NaCl, 1% TRITON X-100, 0.5% NaDeoxycholate, 0.1% SDS, 1 mM EDTA, 2 mM Naf and 1 protease inhibitor) and centrifuged at 20,000 g for 15 minutes. The supernatant of centrifuged lysates was incubated with Streptavidin dynabeads for 2 hours. Bead complexes were washed in lysis buffer and then TBS. Proteins were eluted in 2 X Laemmli sample buffer before western blotting.

### Results

### TAT-P₄-(C5)₂, but not TAT-(C5), Reduces Functional Interaction of PICK1 with AMPAR in Spinal Cord

PICK1 has been evoked as a putative target in treatment of pain. To address pharmacokinetic and -dynamic properties in vivo both peptides were administered intrathecally in mice. TMR-TAT-P₄-(C5)₂ and TMR-TAT-(C5) (20 μM) were clearly visible in both the ventral and dorsal parts of the spinal cord 1 h after injection. Moreover, TMR-TAT-(C5) seemed to distribute better to the interior parts of the spinal cord. Administration of TAT-P₄-(C5)₂ also reduced co-IP of GluA2 by PICK1 from the spinal cord, similar to our observation on hippocampal slices (FIG. 7A). TAT-(C5), however, did not reduce GluA2 co-IP by PICK1 (FIG. 7B), suggesting different pharmaco-dynamic/kinetic properties of the two peptides in spinal cord. Next, we tested the effect of both peptides on GluA2 S880 phosphorylation levels on spinal cord slices and again, in contrast to hippocampal slices, TAT-P₄-(C5)₂, but not TAT-(C5), significantly reduced S880 phosphorylation. To address putative effects on AMPAR surface levels, surface biotinylation experiments were performed on spinal cord slices.

Neither peptides affected GluA1 surface levels whereas both peptides gave rise to a small but non-significant reduction in surface GluA2 levels in animals not subjected to spared nerve injury (FIG. 8).

### Example 6

### Materials and Methods

### Surgical Procedure

Spared nerve injury model (SNI) was performed according to methods described in Richner et al., 2011. Briefly, under isoflurane (2%) anaesthesia, the skin on the left lateral surface of the thigh was incised and the biceps femoris muscle was divided and spread lengthwise to expose the three branches of the sciatic nerve. After exposing the three branches (common peroneal, tibial and sural) in anesthetized mice, the common peroneal and tibial branches were carefully segregated from surrounding tissues, tightly ligated and axotomized ˜2 mm of the distal nerve stump, while the sural branch was left intact.

### Drug Preparation and Administration

Drugs were administered either intrathecally to anesthetized mice or intraperitoneally (i.p.).

### Behavioural Experiments

The development and level of mechanical threshold was determined in the affected hind paws 2 days after the SNI procedure by using Von Frey filaments ranging from 0.02 to 1.4 g. In the current experiments filaments in ascending order were applied to the lateral part of the hind paws. Each Von Frey hair was applied five times over a total period of 30 seconds and the mouse's reaction was assessed after each application; the threshold for a positive test was set at 3 trials, which evoked responses out of a maximum of 5 trials. A positive pain reaction is defined as sudden paw withdrawal, flinching and/or paw licking induced by the filament. Furthermore, a positive response in three out of five repetitive stimuli is defined as the pain threshold. Forces of the instrument are measured with units “g” and represent the gram-forces. The paw withdrawal threshold (PWT) was estimated by using the following formula: PWT=(Number of response failures)/(Total number of trials)×((filament A+1 gr)−(filament A gr))+filament A gr.

### Fluorescence Peptide Administration

10 weeks old SNI male mice were injected with 20 μM TAT-P₄-(C5)₂ intrathecally and trans-cardially perfused with cold phosphate buffer (PBS) at time 0, 30, 60, 120 minutes post-injection. Naïve animals were subjected to the same procedure after intrathecal injection of 20 μM TAT-P₄-(C5)₂ or TAT-(C5) and perfused at time 60 minutes post-injection. All spinal cords were isolated by hydraulic extrusion and post-fixed in PFA 4%. Spinal cords were subsequently cryo-protected in 30% sucrose, embedded in optimal cutting temperature compound (OCT) and sliced.

### Immunohistochemistry

Mounted slides were dried at room temperature, rinsed in PBS1X and incubated in blocking buffer containing 5% Normal goat serum, 1% BSA, 0.3% TritonX-100 in PBS1X, for 90 minutes at room temperature. The slices were incubated in blocking buffer with primary antibody overnight at 4° C. On the second day, sections were rinsed in washing buffer (0.25% BSA, 0.1% Triton X-100 in PBS) and then incubated with secondary Antibodies Alexa-488 or 647. Sections were then washed, air-dried and mounted with DAPI Fluoromount-G mounting media.

### Epifluorescence Microscopy

Epifluorescence microscopy was performed using a Zeiss axio scan.Z1, with a plan-apochromat 10×/0.45 objective (Carl Zeiss). LED light sources were used for excitation of fluorophores and all channels were imaged covering a total of around 6 μm of the 20 μm thick spinal cord slices.

### Antibodies

Western blots, fixed primary hippocampal neurons, HEK 293 cells and tissue sections were analysed with primary antibodies directed against the following: GFAP, NeuN, PICK1, GFP, GluA2, pS880 GluA2, GluA1, Pan Cadherin, β-Actin HRP-conjugated and IgG negative controls. The following secondary antibodies were used: Alexa Fluor 488-conjugated goat anti-rabbit, Alexa Fluor 647-conjugated goat anti-mouse IgG, Alexa Fluor 488-conjugated goat anti-mouse IgG and (HRP)-conjugated secondary antibodies for western blot.

### Results

### TAT-P₄-(C5)₂, but not TAT-(C5), Reduces Surface AMPAR Levels to Alleviate Neuropathic Pain

Peripheral neuropathic pain is produced by multiple etiological factors that initiate a number of diverse mechanisms operating at different sites and at different times. To mimic this disease state, we took advantage of the spared nerve injury model and first tested the effect of the peptides in the acute phase of the model. The SNI model produced a robust hypersensitivity (reduced withdrawal threshold) to mechanical stimuli to the ipsilateral hindpaw (gray) without affecting the contralateral paw (black) (FIG. 9A). Intrathecal administration of TAT-P₄-(C5)₂ two days after operation significantly alleviated this hypersensitivity, with a withdrawal threshold similar to the contralateral paw 1 h after administration, and the effect being significantly different from baseline after 3 h before returning to baseline after 24 h (FIG. 9A). On the other hand, intrathecal administration of TAT-(C5) did not affect withdrawal threshold (FIG. 9B) nor did a myristoylated GluA2 inhibitory peptide or the small molecule inhibitor of PICK1 FSC231. Biotinylation on spinal cord slices showed that both surface GluA1 and GluA2 were increased by the SNI model, and treatment with TAT-P₄-(C5)₂, but not TAT-(C5), significantly reduced the surface level of both GluA1 and GluA2 in the SNI animal to a level below the control (FIG. 9C).

Injection of the TMR labelled TAT-P₄-(C5)₂ in SNI animals initially distributed along the edges of the spinal cord but at later time-points it was also clearly visible in the interior parts. Interestingly, the signal was exclusively seen on neurons (NeuN) and not on glia (GFAP).

The later stage of SNI is thought to better mimic chronic pain, and is notoriously difficult to reverse as is neuropathic pain in humans. Intrathecal administration of TAT-P₄-(C5)₂ significantly increased paw withdrawal threshold at 14 days after operation and again after 28 days (FIG. 10A) with an efficacy comparable to gabapentin (FIG. 10B), suggesting that the peptide may serve as a strong lead for development of efficacious pain therapy.

### Example 7

### Materials and Methods

### Drugs

Cocaine was dissolved in bacteriostatic 0.9% saline. The control peptide TAMRA-C5 corresponds to the conjugation of the TAMRA fluorophore to the distal C5 residues HWLKV of the dopamine transporter (DAT) (SEQ ID NO: 1). TAMRA-YGRKKRRQRRR-PEG4-(HWLKV)₂ (YGRKKRRQRRR_SEQ ID NO: 7), TAMRA-conjugated TAT-P4-(C5)₂ and C5 were dissolved in bacteriostatic 0.9% saline. The doses and time course of PICK1 peptide inhibitor administration were based on previous in vivo rodent studies showing that similar peptides systemically administered readily cross the blood-brain barrier, accumulate in neurons and produce behavioural responses (Bach et al., 2012, Kucharz et al., 2016).

### Materials

Each operant chamber was equipped with both active and inactive response levers, a sucrose pellet dispenser, cue lights, tone generator, as well as an automated injection pump for administering drug or vehicle solutions intravenously. Open field locomotor activity experiments were performed with Photobeam Activity Systems (PAS)-Open Field systems. Locomotor activity monitoring chambers (17″ I×17″ w×15″ h) were surrounded by a photobeam grid with each beam separated by 1″. All beam interruptions were timed stamped per x, y coordinates and sent to a computer for further analysis.

### Surgery

Rats were anesthetized using a mixture of 80 mg/kg ketamine and 12 mg/kg xylazine. An indwelling catheter was inserted into the right jugular vein and sutured in place. The catheter was routed to a mesh backmount platform that was implanted subcutaneously dorsal to the shoulder blades.

After catheter insertion, some rats were immediately mounted in a stereotaxic apparatus and implanted with cannulae for intra-cranial microinjections. Bilateral stainless steel guide cannulae were implanted 2 mm dorsal to the nucleus accumbens shell and cemented in place by affixing dental acrylic to stainless steel screws secured in the skull. The coordinates for the ventral ends of the guide cannulae, relative to bregma were used according to the atlas of Paxinos and Watson (Paxinos, 1997).

### Cocaine Self-Administration, Extinction and Reinstatement of Cocaine Seeking

Rats were allowed 7 days to recover from surgery before behavioral testing commenced. Initially, rats were placed in operant conditioning chambers and allowed to lever-press for intravenous infusions of cocaine (0.25 mg cocaine/59 μl saline, infused over a 5 s period) on a fixed-ratio 1 (FR1) schedule of reinforcement. Rats were allowed to self-administer a maximum of 30 injections per 120 min operant session. Once a rat achieved at least 20 infusions of cocaine in a single daily operant session under the FR1 schedule, the subject was switched to a fixed-ratio 5 (FR5) schedule of reinforcement. The maximum number of injections was again limited to 30 per daily self-administration session under the FR5 schedule. For both FR1 and FR5 schedules, a 20 s time-out period followed each cocaine infusion, during which time active lever responses were tabulated but had no scheduled consequences. Responses made on the inactive lever, which had no scheduled consequences, were also recorded during both the FR1 and FR5 training sessions. Following 21 days of daily cocaine self-administration sessions, drug-taking behavior was extinguished by replacing the cocaine solution with 0.9% saline. Daily extinction sessions continued until responding on the active lever was <15% of the total active lever responses completed on the last day of cocaine self-administration. Typically, it took 5-7 days for rats to meet this criterion. Once cocaine self-administration was extinguished, rats entered the reinstatement phase of the experiment. Rats were infused intravenously through the jugular catheter with vehicle and TAT-P4-(C5)2 (0.3 and 3 nmol/g) 45 min prior to an acute injection of cocaine (10 mg/kg, i.p.). Rats were then placed immediately into the operant conditioning chambers and a two-hour reinstatement session commenced.

During reinstatement test sessions, satisfaction of the response requirement (i.e., five presses on the active lever) resulted in an infusion of saline rather than cocaine. Using a between-sessions reinstatement paradigm, each reinstatement test session was followed by extinction sessions until responding was again <15% of the total active lever responses completed on the last day of cocaine self-administration. Generally, 1-2 days of extinction were necessary to reach extinction criterion between reinstatement test sessions. The effects of intra-accumbens shell infusions of TAT-P4-(C5)2 on cocaine priming-induced reinstatement of drug-seeking behavior were studies in separate cohorts of rats. TAT-P4-(C5)2 (0.3 and 3.0 pmol) and vehicle were microinjected directly into the nucleus accumbens shell 10 min prior to a priming injection of cocaine (10 mg/kg, i.p.). Bilateral infusions into the accumbens shell were performed in a total volume of 500 nl over 2 min. Following infusion, microinjectors were left in place for one additional minute to allow for diffusion of the drug solution away from the tips of the microinjectors. A within-subjects design was used for all experiments in which each rat served as its own control. To control for potential rank order effects of drug and vehicle administrations, all treatments were counterbalanced across reinstatement test sessions.

Cannula Placements were Verified According to Previously Described Protocols

(Schmidt et al., 2009, Schmidt et al., 2016). Briefly, after completion of all intra-cranial microinjection experiments, rats were given an overdose of pentobarbital (100 mg/kg i.p.). Brains were removed and drop fixed in 10% formalin. Coronal sections (100 μm) were taken at the level of the striatum and mounted on gelatin-coated slides. Rats with cannula placements outside of the accumbens shell and/or excessive mechanical damage were excluded from subsequent data analyses.

### Sucrose Self-Administration, Extinction and Reinstatement of Sucrose Seeking

Potential nonspecific rate-suppressing effects and operant learning deficits of intra-accumbens shell TAT-P4-(C5)2 were evaluated by assessing the influence of TAT-P4-(C5)2 on the reinstatement of sucrose-seeking behavior. Separate cohorts of rats were trained initially to self-administer 45 mg sucrose pellets on a FR1 schedule of reinforcement during daily one hour operant sessions. Once rats achieved stable responding for sucrose (defined as <20% variation in responding over 3 consecutive days) on the FR1 schedule of reinforcement, the response requirement was increased to an FR5 schedule of reinforcement. Rats were limited to 30 sucrose pellets within each daily operant session and were restricted to ˜25 g of lab chow daily in their home cages for the duration of the experiment.

After two weeks of sucrose-maintained responding on an FR5 schedule of reinforcement, rats underwent an extinction phase where active lever pressing no longer resulted in sucrose delivery. Once active lever responding decreased to <15% of the maximum number of responses completed on the last day of sucrose self-administration, rats proceeded to reinstatement testing. To determine the effects of systemic TAT-P4-(C5)₂ on sucrose seeking, rats were pretreated with vehicle and 3.0 nmol/g TAT-P4-(C5)₂ (i.v.) 45 min prior to sucrose reinstatement test sessions. Separate groups of rats were used to study the effects of intra-accumbens shell infusions of TAT-P4-(C5)₂ on sucrose seeking. Vehicle and TAT-P4-(C5)₂ (0.3 and 3.0 pmol/μl) were microinjected bilaterally into the accumbens shell 10 min prior to the beginning of the reinstatement test sessions. A within-subjects design was used for all sucrose studies with each rat serving as its own control. Doses were counterbalanced across test sessions. The experimenter remotely administered one sucrose pellet every two min for the first 10 min of each reinstatement test session. A between-session paradigm was used so that each daily reinstatement session was followed by an extinction session the following day until responding was again <15% of the total active lever responses maintained by sucrose.

### Locomotor Testing

The effects of systemic TAT-P4-(C5)₂ on locomotor activity were evaluated in cocaine-experienced rats whose drug-taking behavior had been extinguished. Rats were habituated to the locomotor testing chambers for 1 hour daily over three consecutive days. On subsequent testing days, rats received intravenous infusions of vehicle and 3.0 nmol/g TAT-P4-(C5)₂ 45 min prior to an acute injection of cocaine (10 mg/kg, i.p.). Using a within-subjects design, each animal served as its own control and doses were counterbalanced across test sessions. Spontaneous activity in the x-y plane was recorded for 1 hour post injection. Photobeam interruptions/breaks were quantified over 10 min intervals and used as a measurement of locomotor activity.

### Results

### Systemic Administration of the PICK1 Inhibitor TAT-P4-(C5)2 Dose-Dependently Attenuates Cocaine, but not Sucrose, Seeking in Rats

Total lever responses for rats pretreated i.v. with vehicle or 3.0 nmol/g TAT-P4-(C5)2 prior to a cocaine priming-induced reinstatement test session are shown in FIG. 11A. The data reveals significant main effects of treatment and lever as well as a significant interaction between lever and treatment. Subsequent post-hoc analyses identified significant differences in responding on the active lever between rats pretreated with vehicle and 3.0 nmol/g TAT-P4-(C5)₂. A dose-response curve was conducted in a separate cohort of rats. Total lever responses for rats pretreated i.v. with TAT-P4-(C5)₂ (0, 0.3 and 3.0 nmol/g) prior to a cocaine priming-induced reinstatement test session are shown in FIG. 11B. The experiment revealed significant main effects of treatment and lever as well as a significant interaction between lever and treatment. Subsequent post-hoc analyses indicated that active lever responses were significantly different between vehicle and 3.0 nmol/g TAT-P4-(C5)2, but not 0.3 nmol/g peptide. No significant effects of drug treatment were found on inactive lever responding in either experiment.

While systemic administration of TAT-P₄-(C5)₂ did not affect inactive lever responding, one could argue that responses on the inactive lever were too low to assess the potential rate-suppressing effects of systemic TAT-P4-(C5)₂ administration. Therefore, reinstatement of sucrose seeking was assessed in a separate cohort of rats pretreated with the behaviourally relevant dose of TAT-P4-(C5)2 to attenuate cocaine seeking (3.0 nmol/g). No effects of TAT-P4-(C5)2 were found on sucrose seeking (FIG. 12A) indicating that TAT-P4-(C5)2 attenuates cocaine seeking and that these effects are not due to deficits in operant responding.

### Systemic Administration of the PICK1 Inhibitor TAT-P4-(C5) 2 does not Affect Locomotor Activity

To verify that systemic infusions of TAT-P4-(C5)₂ did not produce general motor impairments; we assessed the locomotor activity of cocaine-experienced rats treated with the PICK1 inhibitor. On the experiment day, rats were injected with 3 nmol/g peptide 45 min prior of an i.p. injection of 10 mg/kg cocaine and placement in the testing chambers. Total beam breaks for each 10 min interval of the 60 min test session and the entire test session are shown in FIGS. 12B and 12C, respectively, for rats pretreated with systemic TAT-P4-(C5)₂ or vehicle. There were no significant effects of drug treatment on locomotor activity.

Taken together, these studies identify a systemic dose of TAT-P4-(C5)2 (i.e., 3.0 nmol/g) that reduces reinstatement of cocaine seeking without inducing motor suppressant effects or operant learning deficits.

### Example 8

### Materials and Methods

### Immunohistochemistry and Blood Brain Barrier Permeability

Immunohistochemical analyses were performed to determine if systemic TAT-P4-(C5)₂ were able to penetrate the brain. Using a between-subjects design, rats were treated acutely with 3.0 nmol/g TAMRA-conjugated TAT-P4-(C5)₂ (i.v.) once cocaine self-administration had been extinguished. Rats were anesthetized and transcardially perfused with 0.1 M PBS (pH 7.4) followed by 4% formalin in 0.1 M PBS 15, 45 or 90 min post infusion of TAMRA-conjugated TAT-P4-(C5)₂. Brains were then removed, postfixed in 4% formalin and then cryoprotected in 20% sucrose in 0.1 M PBS at 4° C. for three days. Coronal sections (30 μm) were taken at the level of the striatum using a cryostat. Brain sections were stored in 0.1 M PBS at 4° C. until processing.

Immunohistochemistry was performed on free-floating coronal sections containing the nucleus accumbens according to modified procedures from previously published studies (Schmidt et al., 2016, Hernandez et al., 2018). Briefly, sections were washed with 1% sodium borohydride followed by 0.1 M PBS. Sections were then blocked in 0.1 M PBS containing 5% normal donkey serum and 0.2% Triton-X for 1 hour at room temperature. Sections were incubated in primary antibodies overnight, and then, following a PBS rinse, were incubated in secondary antibodies for 2 hours. The primary antibodies used were rabbit anti-NeuN (1:1000) and goat anti-GFAP (1:1000). The secondary antibodies used were donkey anti-rabbit Alexa Fluor 488 (1:500) and donkey anti-goat Alexa Fluor 647 (1:500). Sections were washed and mounted onto glass slides and coverslipped using Vectashield with DAPI. Sections were visualized with a Leica SP5 X confocal microscope.

### Neonatal Rat Striatal Culture Preparation and Transduction

Brains from prenatal E19 rats were dissected and placed in ice-cold dissection media (HBSS) supplemented with 30 mM glucose, 10 mM HEPES (pH 7.4, Gibco), 1 mM sodium pyruvate, 100 U/ml penicillin and 10 mg/ml streptomycin and cut in half by sagittal incision. Using a dissection microscope, the striatal compartment were punched out by using a Pasteur pipette followed by removal of cortex by using a scalpel. The striatum was treated with papain at 37° C. for 20 min, triturated and filtered to remove cell debris. The cells were seeded on acid treated poly-L-lysine coated 15 mm coverslips at a density of 130,000 cells/well in Neurobasal media supplemented with 5% FBS, 2% B27 supplement, 1:1000 Glutamax, 25 μM glutamate, and 100 U/ml penicillin streptomycin. After 24 h from the seeding, the growth media was replaced with Neurobasal media supplemented only with 2% B27, 1:1000 Glutamax and 100 U/ml penicillin streptomycin for up to 12-14 days in vitro (DIV). The media was changed every 4 days and 9 days after the transfection procedure 5-fluor-2′-deoxyuridine was added to the serum and glutamate free media.

For shRNA-mediated knock down and replacement studies of PICK1, striatal neurons were transduced at 14 DIV with three different lentivirus; FUGWH1sh18GFPPICK1 (GFP-PICK1WT), FUGWH1sh18eGFP (GFP-sh18) or FUGWH1sh18deleGFP (GFP). The replacement plasmid GFP-PICK1WT expresses a short hairpin (sh18) that targets endogenous PICK1 and a resistant eGFP tagged PICK1. The GFP control vector (GFP) was created by removing the short hairpin (Holst et al., 2013, Jensen et al., 2018). Lentiviruses were produced as described previously (Rasmussen et al., 2009).

### Immunocytochemistry

At 20-22 DIV striatal neurons were incubated with 5 nM of TAMRA-labelled TAT-P4-(C5)₂ (TMR-TAT-P4-(C5)₂) for 1 hour at 37° C., rinsed in PBS and fixed in 4% PFA+4% sucrose. Neurons were rinsed in PBS and blocked in 0.05% Triton-X100 with 5% goat serum for 20 min-at room temperature. Subsequently, neurons were labelled with rabbit anti-DARRP32 (1:800) and chicken anti-GFP (1:2000) for 1 hour at room temperature, before incubation with 1:500 goat anti-rabbit Alexa-647 and goat anti-chicken Alexa-488 secondary antibodies. After three final washes with PBS the coverslips were mounted by using Prolong Gold Antifade™ mounting medium.

For cell penetration studies, 14 DIV striatal neurons were treated with 5 μM of TMR-TAT-P4-(C5)₂ for 1 hour at 37° C., rinsed 3 times in PBS and incubated with 5 μM of the membrane dye DiO for 10 minutes at room temperature. After 3 additional washes in PBS, the striatal neurons were fixed in 4% PFA+4% sucrose. Following a brief wash in PBS, the coverslips were mounted by using Prolong Gold Antifade™ mounting.

Confocal microscopy

Permeability images were acquired using a Zeiss LSM 510 confocal laser-scanning microscopy.

Images for quantification of peptide were acquired with a Zeiss LSM 710 laser-scanning microscopy. Quantification of the TMR-TAT-P4-(C5)₂ intensity was performed by defining each neuron as a region of interest. The mean intensity values obtained from each neuron were subsequently normalized to the average of the intensity values of the GFP transduced neurons

### Results

### TAMRA-Conjugated TAT-P4-(C5)2 Administered Peripherally Penetrates the Brain and was Visualized in the Nucleus Accumbens Shell

To determine if systemically administered TAT-P4-(C5)₂ reaches the brain, rats were pretreated with systemic TAMRA-conjugated TAT-P4-(C5)₂ and sacrificed 15, 45 or 90 minutes post infusion. Confocal microscopy revealed TAMRA-conjugated TAT-P4-(C5)₂ (bright punctae as indicated by arrow heads) located in proximity to GFAP-positive astrocytes and NeuN-positive neurons in the nucleus accumbens shell at all time points as shown in FIG. 13. Overall, these data reveal that systemically administered TAMRA-conjugated TAT-P4-(C5)₂ crosses the blood brain barrier and is visualized in the accumbens shell at time points that coincide with the reduced cocaine-seeking behavior.

These results suggest that the suppressive effects of systemic TAT-P4-(C5)₂ on the reinstatement of cocaine seeking may be due, in part, to inhibition of PICK1 in the nucleus accumbens shell.

### TAMRA-Conjugated TAT-P4-(C5)₂ Penetrates Striatal Neurons in Culture and is Retained by PICK1

To directly address the cell membrane permeability of TAT-P4-(C5)₂, we conjugated TAMRA to the N-terminus of the TAT sequence. Striatal neurons were treated for 1 hr with either TAT-P4-(C5)₂ or the C5 alone (5 μM), both conjugated to TAMRA for visualization, followed by DiO labelling of the plasma membrane. TMR-C5 did not give any fluorescent signal as expected, whereas TMR-TAT-P4-(C5)₂ demonstrated a strong fluorescent labelling of the neurons.

Next, we addressed whether the accumulation of peptide in medium spiny neurons was related to the expression of PICK1. A virally encoded shRNA targeting PICK1 (sh18) coupled to GFP (GFP-sh18) (Citri et al., 2010) was expressed and indeed the TMR-TAT-P4-(C5)₂ signal in GFP and DARPP-32 positive neurons was a significantly reduced compared to neurons not expressing GPF without sh18 (GFP). Finally, expression of sh18 together with a shRNA insensitive GFP-PICK1 (GFP-PICK1) significantly increased the TMR-TAT-P4-(C5)₂ signal compared to control (GFP) despite a relatively low evident overlay of the fluorescent signals.

Taken together, these experiments suggest that TMR-TAT-P4-(C5)2 permeates the plasma membrane and that the accumulation in medium spiny neurons is related to expression of the target PICK1.

### Administration of TAT-P4-(C5)2 Directly into the Nucleus Accumbens Shell Dose-Dependently Attenuates Cocaine, but not Sucrose, Seeking in Rats

Having shown that systemic TAT-P4-(C5)₂ penetrates the brain and is visualized in the accumbens shell, we next investigated whether infusing TAT-P4-(C5)₂ directly into the accumbens shell would block the reinstatement of cocaine seeking. Total lever responses for rats pretreated with vehicle and TAT-P4-(C5)₂ (0.3 and 3.0 pmol/μl) in the shell prior to a cocaine priming-induced reinstatement test session. The data revealed significant main effects of treatment and lever as well as a significant interaction between lever and treatment. Subsequent post-hoc analyses revealed significant differences in responding on the active lever between rats pretreated with vehicle and 0.3 pmol/μl TAT-P4-(C5)₂. There were no significant effects of drug treatment on inactive lever responding. To rule out any potential rate-suppressing effects, we assessed the ability of intra-accumbens shell TAT-P4-(C5)₂ to attenuate sucrose reinstatement in a separate cohort of rats. No effects of TAT-P4-(C5)₂ infusions into the accumbens shell were found on sucrose seeking.

Together, these results identify an important role for PICK1 in the accumbens shell in cocaine seeking.

### Example 9

### Variants of CPP

A cell-penetrating peptide (CPP), linked to the dimeric PICK1 inhibitors of the invention, is introduced in order to improve the transport of the inhibitor across the blood brain barrier and the plasma membrane of target neurons. So far, only the Tat sequence, an 11-mer CPP sequence (YGRKKRRQRRR) derived from the human immunodeficiency virus-type 1 (HIV-1) Tat protein, which facilitates permeability has been tested in vitro and in vivo.

To expand the view on the properties of alternative CPPs, we substituted the cell-penetrating sequence TAT (Trans-activator of Transcription) of TAT-P₄-(C5)₂ peptide by alternative cell-penetrating peptides; Penetratin (PNT), Transportan10 (TP10) and Model Amphipathic peptide (MAP). We hypothesized, that these peptides should obtain similar or increased affinity for PICK1 determined by fluorescence polarization and achieve similar or better analgesic effect in the spared nerve injury neuropathic pain model.

FIG. 14 shows the schematic structure of the tested peptides; TAT-P₄-(C5)₂, PNT-P₄-(C5)₂, TP10-P₄-(C5)₂, and MAP-P₄-(C5)₂. The cell-penetrating peptide sequence TAT, PNT, TP10 or MAP conjugated to 4 polyethylene glycol NPEG-linker N-terminally fused to the bivalent penta-peptide DATC5-the last 5 a.a residues (HWLKV: SEQ ID NO: 1) of DAT.

Penetratin (PNT) (SEQ ID NO: 12) is a 16 a.a. sequence, derived from a 60 a.a. residue Antennapedia homeodomain (DHAntp) from Drosophilia. This 16 a.a. sequence, belonging to the third α-helix of the DHAntp, corresponding to residue 43-58, is the domain facilitating transporting across the membrane.

Transportan10 (TP10) (SEQ ID NO: 13) is a truncated analogue of Transportan, synthesized by deletion of six a.a. from the N-terminus. The internalization mechanism of TP10 is suggested to involve peptide binding to the cellular surface, creating a local positive curvature and a mass imbalance across the bilayer which strains the membrane resulting in pore formation and finally translocation across the membrane.

MAP (SEQ ID NO: 14) the “Model of Amphipathic Helix” is an artificial sequence and designed to have cell-penetrating properties.

### Materials and Methods

PICK1 was expressed and purified as described in example 1.

### Fluorescence Polarization

The competition binding assay was carried out using a fixed concentration of PICK1 (0.25 μM) and fluorescent tracer (5 nM) 5-FAM-TAT-C5 incubated with increasing concentrations of unlabelled peptides, TAT-P₄-(C5)₂, PNT-P₄-(C5)₂, TP10-P₄-(C5)₂, MAP-P₄-(C5)₂, using black half-area Corning non-binding surface 96 well plates (Sigma-Aldrich, Ref. no. 3686). The plates were incubated 30-40 min on ice and the fluorescence polarization was measured on an Omega POLARstar plate (BMG LABTECH) reader using excitation filter at 485 nm and long pass emission filter at 520 nm. The data was plotted in GraphPad Prism 7.0 and fitted to a ‘One site-Fit’ Kᵢ curve and the apparent affinities (Kᵢ) for the unlabelled peptides were determined using correction for depletion.

Fast protein liquid chromatography-FPLC: Each peptide was diluted into PBS to a final concentration of 200 μM for TP10-P₄-(C5)₂, MAP-P₄-(C5)₂, PNT-P₄-(C5)₂ respectively, while TAT-P₄-(C5)₂ was diluted to 300 μM and P₄-(C5)₂ was diluted to 1 mM final concentration. All samples were run in a 200 μL volume on a Superdex 200 Increase 10/300 column on an AKTA purifier FPLC system. The absorbance at 280 nm as a function of elution volume was normalized to the maximal absorbance of each peptide and plotted using GraphPadPrism 7.0.

### Spared Nerve Injury Neuropathic Pain Model

Mice: 8 weeks old C57BI6J mice (from Charles River)

Treatment: TAT-P₄-(C5)₂, PNT-P₄-(C5)₂, TP10-P₄-(C5)₂ and MAP-P₄-(C5)₂ diluted in 0.9% saline. 10 μmol/kg peptide solution was subcutaneously administered (100 μL/10 g).

Spared Nerved Injury surgery: The surgery was performed on anaesthetized mice. A complete ligature and transection of the common peroneal and tibial distal branches of the sciatic nerve was performed, leaving the sural branch intact. The operated side is referred to as the ipsilateral side and the non-operated side is referred to as the contralateral side. 7 days post-surgery, a decrease of threshold response to Von Frey filaments of ipsilateral hind paw was observed.

Mechanical threshold response: The mechanical threshold response of the operated mice was measured with calibrated Von Frey filaments using up/down method, and the 50% threshold (g) was calculated (see equation 1). Mechanical pain responses are defined as paw withdrawal, flinching and/or paw licking following filament prick. The experimenter was blinded to mice treatment. One baseline measure was performed before surgery, one measure 7 days after surgery (D+7) before drug's administration. One measure was performed 1 h, 2 h, 3 h and 4 h post drug administration. n=8 mice/timepoint for peptide treatment.

$$50 \% threshold ( g ) = \frac{1 {0}^{[ X + kd ]}}{10000} \qquad Equation 1$$

X: The value (in log units) of the final von Frey filament used
k: tabular value for the response pattern
d: average increment (in log units) between filaments

Statistical analysis: Statistical analysis was performed using Graphpad Prism 7.0. Pain threshold, measured as paw withdrawal threshold, was compared between time “before drug effect” and time points after drug administration (+1, +2, +3, +4 hrs), or against vehicle control (all time points). Two-way RM ANOVA followed by Dunnet's multiple comparison tests. Significance level set to p<0.05.

### Results

### Fluorescent Polarization

Fluorescent polarization (FP) experiments were performed to determine binding affinity for PICK1.

Competition experiment using 5-FAM-TAT-C5 as fluorescent tracer demonstrated the highest affinity for PNT-P₄-(C5)₂ 2.4 nm/1.7-fold shift compared to TAT-P₄-(C5)₂ affinity (4.0 nM). MAP-P₄-(C5)₂ and TP10-P₄-(C5)₂ showed a lower affinity for PICK1 (5.1 nM/0.8-fold shift and 7.8 nM/0.5-fold shift, respectively) (Table 1 and FIG. 15). Based on FP results, the CPP-peptides all demonstrated high affinity for the PDZ domain of PICK1. Overall, comparison of the CPP-peptides PICK1 affinity with TAT-P₄-(C5)₂ shows maintenance of high affinity, ranking the peptides among the most potent PDZ domain inhibitors.

TABLE 1
Determined affinity (Kᵢ) values (nM) from the ‘One
site-Fit’ Kᵢ curve (FIG. 15) for the unlabelled
peptides calculated in GrapPad Prism 7.0.

| | TAT-P₄- | PNT-P₄- | TP10-P₄- | MAP-P₄- |
|---|---|---|---|---|
| | (C5)₂ | (C5)₂ | (C5)₂ | (C5)₂ |
| Affinity (Kᵢ) nM | 4.0 | 2.4 | 7.8 | 5.1 |
| Fold affinity change | 1 | 1.7 | 0.5 | 0.8 |

### Fast Protein Liquid Chromatography-FPLC

Size exclusion chromatography was done in order to evaluate the in-solution behavior of the different CPP variants. Elution volumes suggested that MAP-P₄-(C5)₂ forms higher oligomeric structures than P4-(C5)2, due to its lower elution volume, while the elution volume of TP10-P₄-(C5)₂, Pent-P₄-(C5)₂ and TAT-P₄-(C5)₂ all have a lower elution volume than P₄-(C5)₂ suggests smaller oligomers than P₄-(C5)₂ or direct interaction with the column material (FIG. 16).

### Analgesic Effect of the CPP-Peptides in the SNI Pain Model

An overview of the experimental setup is shown in FIG. 17.

### Analgesic Effect: Comparison to Levels Before Drug Administration

As shown in FIG. 18, TP10- and MAP-peptide treated SNI mice showed a significant increase in ipsilateral paw mechanical threshold 2 h after 10 μmol/kg s.c administration (TP10: p=0.016, MAP: p=0.0081) compared to before drug administration. SNI mice treated with MAP also showed a significant increase in ipsilateral mechanical threshold 1 h after administration (MAP: p=0.019) compared to before drug administration. TAT- and PNT-peptide treated SNI mice did not show significant increase in ipsilateral paw mechanical threshold at any time-point after 10 μmol/kg s.c administration compared to before drug administration. However, a strong trend was observed.

### Analgesic Effect: Comparison to Vehicle Treatment

As shown in FIG. 19, TAT-, TP10- and MAP-peptide treated SNI mice showed a significant increase in ipsilateral paw mechanical threshold response 2 h after 10 μmol/kg s.c administration (TAT: p=0.04, TP10: p=0.0004, MAP: p=0.0080) compared to vehicle treated SNI mice. PNT-peptide treated SNI mice showed no significant increase in ipsilateral paw mechanical threshold at any time-point after 10 μmol/kg s.c administration compared to the vehicle treated SNI mice. The TAT peptide had the longest analgesic effect after s.c administration compared to the other peptides (p=0.029 at 3h).

### Example 10

### Variants of PEG and Variants of Attachment Site of PEG to Peptide

In this series of experiment, we wanted to test the affinity of various PEGx (x=0-4 ethylene glycol moieties) containing dimeric PICK1 inhibitors (FIG. 20) towards purified PICK1 in the fluorescence polarization binding assay.

### Materials and Methods

PICK1 was expressed and purified as described in example 1.

Fluorescence polarization: Fluorescence polarization was carried out in competition mode at a fixed concentration of protein and tracer (5FAM-PEG4-(DATC5)₂, 5 nM), against an increasing concentration of unlabelled bivalent PEGx-(C5)₂. The plate was incubated 2-4 hrs on ice in a black half-area Corning Black non-binding surface 96-well plate and the fluorescence polarization was measured on an Omega POLARstar plate reader using excitation filter at 488-nm and long pass emission filter at 535-nm. The data was plotted using GraphPad Prism 6.0, and fitted to the One site competition, to extract KI values, which were all correlated to the WT C5 affinity, which was finally plotted as fold affinity increase.

### Results

To test the minimal distance required between the two identical 5-mer peptides, we decided to test different PEG linker lengths. All distances imposed by the PEG linker were tolerated possibly with PEG₀ increasing affinity modestly.

### Example b 11

### Variants of the PDZ-Binding Peptide (DAT-C5, SEQ ID NO: 1))

To test the stringency of the PICK1 PDZ binding motif in the DAT C5 sequence (i.e. position X₁-X₅) and to indicate putatively peptides with better affinity, we performed fluorescence polarization binding of 12 different C5 peptides to purified PICK1 with each residue in the HWLKV sequence (SEQ ID NO: 1) substituted to selected amino acids.

### Materials and Methods

PICK1 was expressed and purified as described in example 1.

Fluorescence polarization: Fluorescence polarization was carried out in competition mode at a fixed concentration of protein and tracer (5FAM-DATC5, 20 nM), against an increasing concentration of unlabelled C5 peptide with point substitutions as indicated. The plate was incubated 20 min on ice in a black half-area Corning Black non-binding surface 96-well plate and the fluorescence polarization was measured directly on an Omega POLARstar plate reader using excitation filter at 488-nm and long pass emission filter at 535-nm. The data was plotted using GraphPad Prism 6.0, and fitted to the One site competition, to extract Kd values, which were All correlated to the WT C5 affinity, which was finally plotted.

### Results

Substitution at X5 and X3 was mostly disruptive to binding except for substitution to V and I on P-2, which increased affinity (FIG. 22). On X4, substitutions to R increased affinity. Substitution to F in position X2, resulted in slightly reduced affinity. Substitutions in position X1 to A or I resulted in increased affinity, whereas substitution to L reduced the affinity.

### Example 12

### Plasma Stability

The stability of DAT-C5, TAT-DAT-C5 (monomer) and TAT-diC5 (dimer) was tested in plasma and PBS.

### Materials and Methods

360 μL undiluted human male serum (Sigma; H4522) or PBS was kept at 37° C. for 15 min, followed by addition of 40 μL (500 uM) of relevant peptide and subsequent incubation at 37° C. For stability analysis, 45 μL samples was taken out at t=0 min and t=24 hrs, and samples quenched by addition of 50 μL 6M urea followed by incubation at 37° C. for additional 30 min. 50 μL 20% TCA (W7V) in acetone was added to each sample before incubation for at least 16 hrs at 5° C. Samples were centrifuged at 14.000×g for 30 min and supernatants collected and filtered using a 0.22 μm syringe filter. Samples were analyzed on an Acquity UPLC H-Class instrument using a C18 column with a 15 min 10-80% TCA gradient. Remaining analyte (%) was calculated as the area under curve (AUC) of the main peak at indicated time points relative to the sample at t=0 min.

### Results

As shown in FIG. 23, all three peptides, DAT-C5, TAT-C5 (monomer) and TAT-P4-(C5)₂ (dimer) possessed good stability in PBS. In contrast, full degradation of DAT-C5 in human plasma was observed after 24 h incubation. The plasma stability was improved for TAT-DAT-C5 (monomer) and TAT-P4-(C5)₂ (dimer), with TAT-P4-(C5)₂ showing the highest plasma stability.

### Example 13

### Efficacy Study of TAT-P4-(C5)₂ in a Model of Inflammatory Pain

Assessment of the efficacy of a single i.t. administration of TAT-P4-(C5)₂ to relieve inflammatory pain, induced by administration of Complete Freund's Adjuvant (CFA).

### Materials and Methods

Animals; 5-6 male C57BL6/N mice (SPF status, Janvier, France) of 8 weeks of age at beginning of experiment were used in each group. Mice were allowed at least 7 days of habituation to our facility before initiation of experiment. Mice were group-housed in IVC-cages in a temperature-controlled room maintained on a 12:12 light: dark cycle (lights on at 6 AM) and allowed access to standard rodent chow and water ad libitum.

Drug; TAT-P4-(C5)₂ was dissolved (final concentration 20 uM) in saline prior to the testing. The compound was delivered by i.t. administration under isofluorane anesthesia 60 min prior to the von Frey test.

Induction of inflammatory pain; Inflammatory pain was induced by the use of Complete Freund's adjuvant (CFA). Mice were placed under very light isoflurane anesthesia. The right hindpaw of the mice was sterilized with ethanol, and 5 μL of CFA was injected intraplantar to the right hindpaw with an insulin needle. Mice woke up within seconds of being removed from the isoflurane, and were left for 48 hours while inflammatory pain developed. The development and level of mechanical hyperalgesia/allodynia was determined in the affected hind paws 2 days after the CFA procedure by using Von Frey filaments ranging from 0.04 to 2 g. The filaments are applied to the underside of the paw after the mouse has settled into a comfortable position within a restricted area that has a perforated floor. The filaments are calibrated to flex when the set force is applied to the paw. Filaments are presented in order of increasing stiffness, until a paw withdrawal is detected. In the current experiments, filaments in ascending order were applied to the central part of the hind paws. Each Von Frey hair was applied five times over a total period of 30 seconds and the mouse's reaction was assessed after each application; the threshold for a positive test was set at 3 trials, which evoked responses out of a maximum of 5 trials. A positive pain reaction is defined as sudden paw withdrawal, flinching and/or paw licking induced by the filament. The non-injected left hindpaw was used as an unaffected control.

Measuring the effect of TAT-P4-(C5)2 on Pain Threshold; On the test-day a baseline test was performed to confirm hyperalgesia in the CFA injected animals. Thereafter, saline (vehicle) or 20 uM TAT-P4-(C5)₂ was injected i.t. under isoflurane anesthesia and the mice left in their home-cage for 30 min. Mice were next placed in the confined von Frey boxes for 30 min, and their pain threshold measured by von Frey at 1 hour, 5 hours and 24 hours after injection of TAT-P4-(C5)₂.

### Results

In the CFA model of inflammatory pain, delivery of 20 uM TAT-P4-(C5)₂ significantly increased the paw withdrawal threshold, as seen 1 and 5 hours after i.t. drug administration (FIG. 24). The paw withdrawal threshold remained unaltered in the uninjured contralateral paw. Statistics were done using two-way ANOVA (interaction: F(12,75)=4.752; Time: F(4,75)=12.33; Treatment: F(3,75)=54.10). Post-Bonferroni analysis presented in the figure revealed significant pain relief at both 1 hour (p=0.05) and 5 hours (p=0.0035) after i.t. administration of TAT-P4-(C5)₂, with the pain relief being gone after 24 hours (p>0.9999) and no effect of the vehicle treatment on pain relief (p₁ₕᵣ=0.9979, p₅ₕᵣₛ>0.9999, p₂₄ₕᵣₛ>0.9999)

### Sequences

(DAT-C5, DATC5 or C5)
SEQ ID NO: 1
HWLKV
SEQ ID NO: 2
X₁X₂X₃X₄X₅

wherein:
X₁ is a proteogenic or non-proteogenic amino acid, preferably H, L, I, or A; or is absent;
X₂ is a proteogenic or non-proteogenic amino acid, preferably W, or F; or is absent;
X₃ is a proteogenic or non-proteogenic amino acid, preferably L, V, I, F; A, or Y;
X₄ is a proteogenic or non-proteogenic amino acid, preferably K, or R;
and X₅ is V, I or C

SEQ ID NO: 3
X₁X₂X₃X₄X₅

wherein:
X₁ is H, L, I, A; or is absent;
X₂ is W, F; or is absent;
X₃ is L, V, I, F; A, or Y;
X₄ is K or R; and
X₅ is V, I or C.

SEQ ID NO: 4
X₁X₂X₃X₄X₅

wherein:
X₁ is H, L, I, or A;
X₂ is W or F;
X₃ is L, V, I, F; A, or Y;
X₄ is K or R; and
X₅ is V, I or C.

SEQ ID NO: 5
X₁X₂X₃X₄X₅

wherein:
X₁ is H, I, or A;
X₂ is W or F;
X₃ is L, I or V;
X₄ is K or R;
and X₅ is V or
C.

SEQ ID NO: 6
X₁X₂X₃X₄X₅

wherein:
X₁ is H or A;
X₂ is W;
X₃ is L, I or V;
X₄ is K or R; and
X₅ is V.

(Human immunodeficiency virus-type 1 (HIV-1) TAT
(TAT or TAT11))
SEQ ID NO: 7
YGRKKRRQRRR
(TAT fragment 1)
SEQ ID NO: 8
RKKRRQRRR
(TAT fragment 2)
SEQ ID NO: 9
GRKKRRQRRRP
(Retroinverso-D-TAT)
SEQ ID NO: 10
rrrqrrkkr
(polyarginine peptide)
SEQ ID NO: 11
RRRRRRRR
(Penetratin (PNT))
SEQ ID NO: 12
RQIKIWFQNRRMKWKK
(Transportan10 (TP10))
SEQ ID NO: 13
GWTLNSAGYLLGKINLKALAALAKKIL
(“Model of Amphipathic Helix” (MAP))
SEQ ID NO: 14
KLALKLALKALKAALKLA
(DAT-C3, DATC3 or C3)
SEQ ID NO: 15
LKV
(DAT-C4, DATC4 or C4)
SEQ ID NO: 16
WLKV
(DAT-C6, DATC6 or C6)
SEQ ID NO: 17
RHWLKV
(DAT-C7, DATC7 or C7)
SEQ ID NO: 18
LRHWLKV
(DAT-C8, DATC8 or C8)
SEQ ID NO: 19
TLRHWLKV
(DAT-C9, DATC9 or C9)
SEQ ID NO: 20
FTLRHWLKV
(DAT-C10, DATC10 or C10)
SEQ ID NO: 21
QFTLRHWLKV
(DAT-C11, DATC11 or C11)
SEQ ID NO: 22
RQFTLRHWLKV
(DAT-C12, DATC12 or C12)
SEQ ID NO: 23
VRQFTLRHWLKV
(DAT-C13, DATC13 or C13)
SEQ ID NO: 24
EVRQFTLRHWLKV

### REFERENCES

ALFONSO, S., et al., Synapto-depressive effects of amyloid beta require PICK1. Eur J Neurosci, 2014. 39(7): p. 1225-33.
ATIANJOH, F.E., et al., Spinal cord protein interacting with C kinase 1 is required for the maintenance of complete Freund's adjuvant-induced inflammatory pain but not for incision-induced post-operative pain. Pain, 2010. 151(1): p. 226-34.
BACH, A et al., 2012. A high-affinity, dimeric inhibitor of PSD-95 bivalently interacts with PDZ1-2 and protects against ischemic brain damage. Proc Natl Acad Sci USA, 109, 3317-22.
BERNADO et al., 2007. Structural characterization of flexible proteins using small-angle X-ray scattering. J Am Chem Soc., 129(17):5656-64.
CHEN, S.R., et al., Nerve injury increases GluA2-lacking AMPA receptor prevalence in spinal cords: functional significance and signaling mechanisms. J Pharmacol Exp Ther, 2013. 347(3): p. 765-72.
CITRI, A. et al., 2010. Calcium binding to PICK1 is essential for the intracellular retention of AMPA receptors underlying long-term depression. J Neurosci, 30, 16437-52.
CLEM, R.L., V. Anggono, and R.L. Huganir, PICK1 regulates incorporation of calcium-permeable AMPA receptors during cortical synaptic strengthening. J Neurosci, 2010. 30(18): p. 6360-6.
CONRAD, K.L., et al., Formation of accumbens GluR2-lacking AMPA receptors mediates incubation of cocaine craving. Nature, 2008. 454(7200): p. 118-21.
DIXON, R.M., J.R. Mellor, and J.G. Hanley, Pick1-mediated glutamate receptor subunit 2 (GLUR2) trafficking contributes to cell death in oxygen/glucose deprived hippocampal neurons. Journal of Biological Chemistry, 2009.
GANGADHARAN, V., et al., Peripheral calcium-permeable AMPA receptors regulate chronic inflammatory pain in mice. J Clin Invest, 2011. 121(4): p. 1608-23.
GARRY, E.M., et al., Specific involvement in neuropathic pain of AMPA receptors and adapter proteins for the GluR2 subunit. Mol.Cell Neurosci., 2003. 24(1): p. 10-22.
HE, J., et al., PICK1 inhibits the E3 ubiquitin ligase activity of Parkin and reduces its neuronal protective effect. Proc Natl Acad Sci USA, 2018. 115(30): p. E7193-E7201.
HERNANDEZ, N. Set al., 2018. Glucagon-like peptide-1 receptor activation in the ventral tegmental area attenuates cocaine seeking in rats. Neuropsychopharmacology.
HOLST, B. et al., 2013. PICK1 deficiency impairs secretory vesicle biogenesis and leads to growth retardation and decreased glucose tolerance. PLOS Biol, 11, e1001542.
JENSEN, K. L. et al., 2018. PICK1-Deficient Mice Exhibit Impaired Response to Cocaine and Dysregulated Dopamine Homeostasis. eNeuro, 5.
JI, R.R., et al., Central sensitization and LTP: do pain and memory share similar mechanisms? Trends Neurosci, 2003. 26(12): p. 696-705.
KANDASAMY, R. and T.J. Price, The pharmacology of nociceptor priming. Handb Exp Pharmacol, 2015. 227: p. 15-37.
KARLSEN, M.L. et al. 2015. Structure of Dimeric and Tetrameric Complexes of the BAR Domain Protein PICK1 Determined by Small-Angle X-Ray Scattering. Structure 23, 1258-70.
KUCHARZ, K., et al., 2016. PSD-95 uncoupling from NMDA receptors by Tat-N-dimer ameliorates neuronal depolarisation in cortical spreading depression. J Cereb Blood Flow Metab.
LORGEN, JØ., PICK1 facilitates lasting reduction of GluA2 concentration in the hippocampus during chronic epilepsy. Epilepsy Res, 2017. 137:25-32
LUSCHER, C. and R.C. Malenka, Drug-evoked synaptic plasticity in addiction: from molecular changes to circuit remodeling. Neuron, 2011. 69(4): p. 650-63.
MADSEN, K.L. et al., 2005. Molecular determinants for the complex binding specificity of the PDZ domain in PICK1. Journal of Biological Chemistry 280, 20539-20548.
MARCOTTE, D.J., et al., Lock and chop: A novel method for the generation of a PICK1 PDZ domain and piperidine-based inhibitor co-crystal structure. Protein Sci, 2018. 27(3): p. 672-680.
MOLLER, A.R., Tinnitus and pain. Prog Brain Res, 2007. 166: p. 47-53.
PAXINOS, G. W., C. 1997. The rat brain in stereotaxic coordinates, New York, Academic Press.
PEKER, S. and A. Sirin, Parallels between phantom pain and tinnitus. Med Hypotheses, 2016. 91: p. 95-97.
PEREZ, J.B. et al. Monitoring the diffusion of single heterotrimeric G proteins in supported cell-membrane sheets reveals their partitioning into microdomains. J Mol Biol 363, 918-30 (2006).
RASMUSSEN, T. N. et al., 2009. A single nucleotide polymorphism in the human serotonin transporter introduces a new site for N-linked glycosylation. Neuropharmacology, 57, 287-94.
RICHNER et al., 2011. The spared nerve injury (SNI) model of induced mechanical allodynia in mice. J Vis Exp. (54). pii: 3092.
RICHNER et al., 2017. Hydraulic Extrusion of the Spinal Cord and Isolation of Dorsal Root Ganglia in Rodents. J Vis Exp. 2017; (119): 55226.
SCHMIDT, H. D. et al., 2009. The limbic circuitry underlying cocaine seeking encompasses the PPTg/LDT. Eur J Neurosci, 30, 1358-69.
SCHMIDT, H. D. et al., 2016. Glucagon-Like Peptide-1 Receptor Activation in the Ventral Tegmental Area Decreases the Reinforcing Efficacy of Cocaine. Neuropsychopharmacology, 41, 1917-28.
TRIA et al., 2015. Advanced ensemble modelling of flexible macromolecules using X-ray solution scattering. IUCrJ. 2015 Feb. 26; 2(Pt 2):207-17.
VANNESTE, S., W.T. To, and D. De Ridder, Tinnitus and neuropathic pain share a common neural substrate in the form of specific brain connectivity and microstate profiles. Prog Neuropsychopharmacol Biol Psychiatry, 2019. 88: p. 388-400.
VIKMAN, K.S., B.K. Rycroft, and M.J. Christie, Switch to Ca2+-permeable AMPA and reduced NR2B NMDA receptor-mediated neurotransmission at dorsal horn nociceptive synapses during inflammatory pain in the rat. J Physiol, 2008. 586(2): p. 515-27.
WANG, W., et al., Preserved acute pain and impaired neuropathic pain in mice lacking protein interacting with C Kinase 1. Mol Pain, 2011. 7: p. 11.
WOLF, M.E. and C.R. Ferrario, AMPA receptor plasticity in the nucleus accumbens after repeated exposure to cocaine. Neurosci Biobehav Rev, 2010. 35(2): p. 185-211.
WOOLF, C.J. and M.W. Salter, Neuronal plasticity: increasing the gain in pain. Science, 2000. 288(5472): p. 1765-9.
ZHANG et al. Protein interacting with C alpha kinase 1 (PICK1) is involved in promoting tumor growth and correlates with poor prognosis of human breast cancer. Cancer Sci. 2010 June; 101(6): 1536-42.

1. A method for treatment of diseases and disorders associated with maladaptive plasticity, the method comprising administration of a PICK1 inhibitor comprising
a) a first peptide comprising an amino acid sequence of the general formula
(SEQ ID NO: 3)
X₁X₂X₃X₄X₅,
b) a second peptide comprising an amino acid sequence of the general formula
(SEQ ID NO: 3)
X₁X₂X₃X₄X₅,
wherein
X₁ is H, L, I, or A; or is absent,
X₂ is W, or F; or is absent,
X₃ is L, V, I, F, A, or Y,
X₄ is K, or R; and
X₅ is V, I or C,
c) an NPEG linker linking the first peptide to the second peptide; and
d) a Cell Penetrating Peptide (CPP)
to a subject in need thereof.

2. The method according to claim 1, wherein the PICK1 inhibitor has a structure according to the formula:

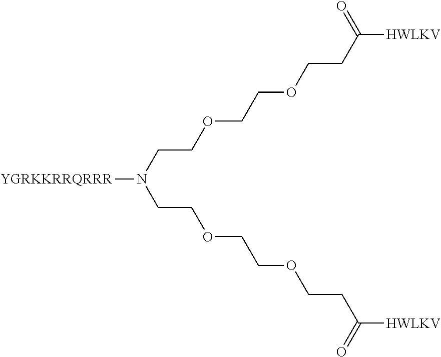

3. The method according to claim 1, wherein the first and/or the second peptide comprise or consist of the amino acid sequence HWLKV (SEQ ID NO: 1).

4. The method according to claim 1, wherein the first and/or second peptide comprises an amino acid sequence of the general formula: X₁X₂X₃X₄X₅ (SEQ ID NO:6):
wherein
X₁ is H, or A;
X₂ is W;
X₃ is L, I or V;
X₄ is K or R; and
X₅ is V.

5. The method according to claim 1, wherein the CPP is conjugated to the nitrogen atom of the NPEG-linker by an amide bond.

6. The method according to claim 1, wherein the NPEG-linker is conjugated to the first and second peptide via an amide bond formed between the carboxylic acids of the NPEG-linker and the N-terminus of the first and/or second peptides.

7. The method according to claim 1, wherein said PICK1 inhibitor has the generic structure of formula: 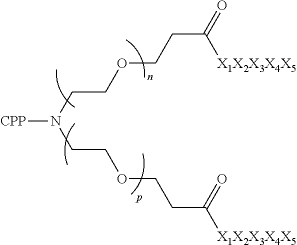
wherein
n is an integer 0 to 12;
p is an integer 0 to 12;
CPP is a cell penetrating peptide.

8. The method according to claim 1, wherein the NPEG-linker comprises in the range of 0 to 12 ethylene glycol moieties wherein one or more of the backbone oxygen atoms is replaced with a nitrogen atom.

9. The method according to claim 1, wherein the CPP comprises a TAT peptide, a Retroinverso-D-TAT peptide, a polyarginine peptide, a PNT peptide, a TP10 peptide or a MAP peptide.

10. The method according to claim 1, wherein the PICK1 inhibitor is capable of inhibiting a protein-protein interaction between AMPAR and PICK1.

11. The method according to claim 1, wherein said PICK1 inhibitor has a Ki for PICK1 inferior to 10 nM.

12. The method according to claim 1, wherein said PICK1 inhibitor is selected from the group consisting of Tat-NPEG₄-(HWLKV)₂, TP10-NPEG₄-(HWLKV)₂, and MAP-NPEG₄-(HWLKV)₂.

13. The method according to claim 1, wherein the first and/or second peptide comprises an ammo acid sequence of the general formula
(SEQ ID NO: 6)
X₁X₂X₃X₄X₅:
X₁ is H, or A,
X₂ is W,
X₃ is L, I or V,
X₄ is K or R; and
X₅ is V.

14. The method according to claim 1, wherein the CPP comprises a TAT peptide, a Retroinverso-D-TAT peptide, a poly arginine peptide, a PNT peptide, a TP10 peptide or a MAP peptide.

15. The method according to claim 1, wherein the CPP is a Tat peptide comprising an amino acid sequence YGRKKRRQRRR (SEQ ID NO: 7) or a Retroinverso-d-Tat peptide comprising the amino acid sequence of RRRQRRKKR (SEQ ID NO: 10).

16. The method according to claim 1, wherein the diseases and disorders associated with maladaptive plasticity is neuropathic pain, inflammatory pain, drug addiction, amyotrophic lateral sclerosis, epilepsy, tinnitus, migraine, ischemia, Alzheimer's disease, or Parkinson's disease.

17. The method according to claim 1, wherein the diseases and disorders associated with maladaptive plasticity is neuropathic pain, drug addiction, amyotrophic lateral sclerosis, epilepsy, tinnitus or migraine.

18. The method according to claim 1, wherein the disease or disorder associated with maladaptive plasticity is inflammatory pain or neuropathic pain.

19. The method according to claim 1, wherein the disease or disorder associated with maladaptive plasticity is neuropathic pain.

20. The method according to claim 1, wherein the disease or disorder associated with maladaptive plasticity is cocaine addiction.


SEQUENCE LISTING (24 sequences)

SEQ ID NO: 1 - protein, 5 aa, synthetic construct
  REGION 1..5: note = DAT-C5, 5 C-terminal residues of human dopamine transporter
HWLKV

SEQ ID NO: 2-6 - skipped (5 entries)

SEQ ID NO: 7 - protein, 11 aa, synthetic construct
  REGION 1..11: note = CPP sequence (TAT) derived from the human immunodeficiencyvirus-type 1 (HIV-1) Tat protein
YGRKKRRQRRR

SEQ ID NO: 8 - protein, 9 aa, synthetic construct
  REGION 1..9: note = TAT fragment 1
  SITE 3: note = MISC_FEATURE - is a proteogenic or non-proteogenic amino acid, preferably H, L,I, or A
  SITE 4: note = MISC_FEATURE - is a proteogenic or non-proteogenic amino acid, preferably W, orF
  SITE 5: note = MISC_FEATURE - is a proteogenic or non-proteogenic amino acid, preferably L, V,I, F, A, or Y
  SITE 6: note = MISC_FEATURE - is a proteogenic or non-proteogenic amino acid, preferably K, orR
  SITE 7: note = MISC_FEATURE - is V, I or C
RKKRRQRRR

SEQ ID NO: 9 - protein, 11 aa, synthetic construct
  REGION 1..11: note = TAT fragment 2
GRKKRRQRRRP

SEQ ID NO: 10 - protein, 9 aa, synthetic construct
  REGION 1..9: note = Retroinverso-D-TAT
  REGION 1..9: note = MISC_FEATURE - D-amino acid
RRRQRRKKR

SEQ ID NO: 11 - protein, 8 aa, synthetic construct
  REGION 1..8: note = Polyarginine peptide
RRRRRRRR

SEQ ID NO: 12 - protein, 16 aa, synthetic construct
  REGION 1..16: note = 16 a.a. sequence (PNT), derived from Antennapedia homeodomain(DHAntp) from Drosophilia, belonging to the third-helix of theDHAntp, corresponding to residue 43-58
RQIKIWFQNRRMKWKK

SEQ ID NO: 13 - protein, 27 aa, synthetic construct
  REGION 1..27: note = Transportan10 (TP10), truncated analogue of Transportan,synthesized by deletion of six a.a. from the N-terminus
GWTLNSAGYLLGKINLKALAALAKKIL

SEQ ID NO: 14 - protein, 18 aa, synthetic construct
  REGION 1..18: note = "Model of Amphipathic Helix" (MAP), an artificial sequence anddesigned to have cell-penetrating properties
KLALKLALKALKAALKLA

SEQ ID NO: 15 - skipped

SEQ ID NO: 16 - protein, 4 aa, synthetic construct
  REGION 1..4: note = DAT-C4, 4 C-terminal residues of human dopamine transporter
WLKV

SEQ ID NO: 17 - protein, 6 aa, synthetic construct
  REGION 1..6: note = DAT-C6, 6 C-terminal residues of human dopamine transporter
RHWLKV

SEQ ID NO: 18 - protein, 7 aa, synthetic construct
  REGION 1..7: note = DAT-C7, 7 C-terminal residues of human dopamine transporter
LRHWLKV

SEQ ID NO: 19 - protein, 8 aa, synthetic construct
  REGION 1..8: note = DAT-C8, 8 C-terminal residues of human dopamine transporter
TLRHWLKV

SEQ ID NO: 20 - protein, 9 aa, synthetic construct
  REGION 1..9: note = DAT-C9, 9 C-terminal residues of human dopamine transporter
FTLRHWLKV

SEQ ID NO: 21 - protein, 10 aa, synthetic construct
  REGION 1..10: note = DAT-C10, 10 C-terminal residues of human dopamine transporter
QFTLRHWLKV

SEQ ID NO: 22 - protein, 11 aa, synthetic construct
  REGION 1..11: note = DAT-C11, 11 C-terminal residues of human dopamine transporter
RQFTLRHWLKV

SEQ ID NO: 23 - protein, 12 aa, synthetic construct
  REGION 1..12: note = DAT-C12, 12 C-terminal residues of human dopamine transporter
VRQFTLRHWLKV

SEQ ID NO: 24 - protein, 13 aa, synthetic construct
  REGION 1..13: note = DAT-C13, 13 C-terminal residues of human dopamine transporter
EVRQFTLRHWLKV